(19) United States
(12) Patent Application Publication
(10) Pub. No.: US 2023/0134375 A1
(43) Pub. Date: May 4, 2023

(54) Anti-TIGIT Antibodies

(71) Applicant: Seagen Inc., Bothell, WA (US)

(72) Inventors: Julia C. Piasecki, Seattle, WA (US); Courtney Beers, Seattle, WA (US); Scott Peterson, Seattle, WA (US); Bianka Prinz, Lebanon, NH (US)

(73) Assignee: Seagen Inc., Bothell, WA (US)

(21) Appl. No.: 17/853,134

(22) Filed: Jun. 29, 2022

Related U.S. Application Data
(63) Continuation of application No. 17/082,586, filed on Oct. 28, 2020, now abandoned.
Application No. 17/082,586 is a continuation of application No. 16/541,575, filed on Aug. 15, 2019, now abandoned.
Application No. 16/541,575 is a continuation of application No. PCT/US2018/020239, filed on Feb. 28, 2018.
(60) Provisional application No. 62/616,779, filed on Jan. 12, 2018.
(60) Provisional application No. 62/464,529, filed on Feb. 28, 2017.

(51) Int. Cl. C07K 16/28; A61K 47/68; A61P 35/00; A61K 45/06

(52) U.S. Cl. CPC C07K 16/2818; A61K 47/6803; A61K 47/6849; A61P 35/00; A61K 45/06; A61K 2039/505

(57) ABSTRACT

Isolated antibodies or antigen-binding portions that bind to human TIGIT (T-cell immunoreceptor with Ig and ITIM domains) are provided. In some embodiments, the antibody or antigen-binding portion thereof has a binding affinity (K_{D}) for human TIGIT of less than 5 nM. In some embodiments, the anti-TIGIT antibody blocks binding of CD155 and/or CD112 to TIGIT.

### CROSS REFERENCE TO RELATED APPLICATIONS

This application is a continuation of U.S. application Ser. No. 17/082,586, filed Oct. 28, 2020, which is a continuation of U.S. application Ser. No. 16/541,575, filed Aug. 15, 2019, now abandoned, which is a continuation of International Application No. PCT/US2018/020239, filed Feb. 28, 2018, which claims priority to U.S. Provisional Patent Application No. 62/464,529, filed Feb. 28, 2017, and to U.S. Provisional Patent Application No. 62/616,779, filed Jan. 12, 2018, the entire contents of each of which are incorporated by reference herein.

### BACKGROUND OF THE INVENTION

TIGIT (“T-cell immunoreceptor with Ig and ITIM domains”) is an immune receptor that is expressed on subsets of T cells, such as activated, memory, and regulatory T cells and natural killer (NK) cells. TIGIT is a member of the CD28 family within the Ig superfamily of proteins, and serves as a co-inhibitory molecule that limits T cell proliferation and activation and NK cell function. TIGIT mediates its immunosuppressive effect by competing with CD226 (also known as DNAX Accessory Molecule-1, or “DNAM-1”) for the same set of ligands: CD155 (also known as poliovirus receptor or “PVR”) and CD112 (also known as poliovirus receptor-related 2 or “PVRL2”). See, Levin et al., Eur. J. Immunol., 2011, 41:902-915. Because the affinity of CD155 for TIGIT is higher than its affinity for CD226, in the presence of TIGIT CD226 signaling is inhibited, thereby limiting T cell proliferation and activation.

In patients with melanoma, TIGIT expression is upregulated on tumor antigen (TA)-specific CD8⁺ T cells and CD8⁺ tumor-infiltrating lymphocytes (TILs). Blockade of TIGIT in the presence of TIGIT ligand (CD155)-expressing cells increased the proliferation, cytokine production, and degranulation of both TA-specific CD8⁺ T cells and CD8⁺ TILs See, Chauvin et al., J Clin Invest., 2015, 125:2046-2058. Thus, TIGIT represents a potential therapeutic target for stimulating anti-tumor T cell responses in patients, although there remains a need for improved methods of blocking TIGIT and promoting anti-tumor responses.

### BRIEF SUMMARY OF THE INVENTION

In one aspect, isolated antibodies or antigen-binding portions thereof that bind to human TIGIT (T-cell immunoreceptor with Ig and ITIM domains) are provided. In some embodiments, the antibody or antigen-binding portion thereof has a binding affinity (K_{D}) for human TIGIT of less than 5 nM. In some embodiments, the antibody or antigen-binding portion thereof has a K_{D} for human TIGIT of less than 1 nM. In some embodiments, the antibody or antigen-binding portion thereof has a K_{D} for human TIGIT of less than 100 pM.

In some embodiments, the antibody or antigen-binding portion thereof exhibits cross-reactivity with cynomolgus monkey TIGIT and/or mouse TIGIT. In some embodiments, the antibody or antigen-binding portion thereof exhibits cross-reactivity with both cynomolgus monkey TIGIT and mouse TIGIT.

In some embodiments, the antibody or antigen-binding portion thereof blocks binding of CD155 to TIGIT. In some embodiments, the antibody or antigen-binding portion thereof blocks binding of CD112 to TIGIT. In some embodiments, the antibody or antigen-binding portion thereof blocks binding of both CD155 and CD112 to TIGIT.

In some embodiments, the antibody or antigen-binding portion thereof binds to an epitope on human TIGIT that comprises amino acid positions 81 and 82. In some embodiments, the epitope comprises Phe at position 81 and/or Lys or Ser at position 82. In some embodiments, the epitope comprises Phe81 and Lys82.

In some embodiments, the epitope is a discontinuous epitope.

In some embodiments, the antibody or antigen-binding portion thereof binds to an epitope on human TIGIT that further comprises one or more of amino acid positions 51, 52, 53, 54, 55, 73, 74, 75, 76, 77, 79, 83, 84, 85, 86, 87, 88, 89, 90, 91, 92, or 93. In some embodiments, the epitope further comprises one or more amino acid residues selected from the group consisting of Thr51, Ala52, Gln53, Val54, Thr55, Leu73, Gly74, Trp75, His76, Ile77, Pro79, Asp83, Arg84, Val85, Ala86, Pro87, Gly88, Pro89, Gly90, Leu91, Gly92, and Leu93. In some embodiments, the epitope comprises the amino acid residues Thr51, Ala52, Gln53, Val54, Thr55, Gly74, Trp75, His76, Ile77, Phe81, Lys82, Pro87, Gly88, Pro89, Gly90, Leu91, Gly92, and Leu93. In some embodiments, the epitope comprises the amino acid residues Ala52, Gln53, Leu73, Gly74, Trp75, Pro79, Phe81, Lys82, Asp83, Arg84, Val85, and Ala86. In some embodiments, the epitope comprises the sequence

(SEQ ID NO: 258)
ICNADLGWHISPSFK.

In some embodiments, the antibody or antigen-binding portion thereof comprises one or more sequences listed in Table 3 below. In some embodiments, the antibody or antigen-binding portion thereof comprises one or more of:
(a) a heavy chain CDR1 comprising the sequence of any of SEQ ID NO:4, SEQ ID NO:22, SEQ ID NO:40, SEQ ID NO:58, SEQ ID NO:76, SEQ ID NO:94, SEQ ID NO: 112, SEQ ID NO:130, SEQ ID NO:148, SEQ ID NO:166, SEQ ID NO:184, SEQ ID NO:202, SEQ ID NO:221, SEQ ID NO:224, SEQ ID NO:226, SEQ ID NO:231, SEQ ID NO:233, SEQ ID NO:239, or SEQ ID NO:243;
(b) a heavy chain CDR2 comprising the sequence of any of SEQ ID NO:6, SEQ ID NO:24, SEQ ID NO:42, SEQ ID NO:60, SEQ ID NO:78, SEQ ID NO:96, SEQ ID NO: 114, SEQ ID NO:132, SEQ ID NO:150, SEQ ID NO:168, SEQ ID NO:186, SEQ ID NO:204, SEQ ID NO:222, SEQ ID NO:225, SEQ ID NO:227, SEQ ID NO:229, SEQ ID NO:232, SEQ ID NO:234, SEQ ID NO:238, or SEQ ID NO:240;
(c) a heavy chain CDR3 comprising the sequence of any of SEQ ID NO:8, SEQ ID NO:26, SEQ ID NO:44, SEQ ID NO:62, SEQ ID NO:80, SEQ ID NO:98, SEQ ID NO:116, SEQ ID NO:134, SEQ ID NO:152, SEQ ID NO:170, SEQ ID NO:188, SEQ ID NO:206, SEQ ID NO:223, SEQ ID NO:228, SEQ ID NO:230, SEQ ID NO:235, SEQ ID NO:236, SEQ ID NO:237, SEQ ID NO:241, SEQ ID NO:242, or SEQ ID NO:244;
(d) a light chain CDR1 comprising the sequence of any of SEQ ID NO:13, SEQ ID NO:31, SEQ ID NO:49, SEQ ID NO:67, SEQ ID NO:85, SEQ ID NO:103, SEQ ID NO:121, SEQ ID NO:139, SEQ ID NO:157, SEQ ID NO:175, SEQ ID NO:193, or SEQ ID NO:211;
(e) a light chain CDR2 comprising the sequence of any of SEQ ID NO:15, SEQ ID NO:33, SEQ ID NO:51, SEQ ID NO:69, SEQ ID NO:87, SEQ ID NO:105, SEQ ID NO: 123, SEQ ID NO:141, SEQ ID NO:159, SEQ ID NO:177, SEQ ID NO:195, or SEQ ID NO:213; or
(f) a light chain CDR3 comprising the sequence of any of SEQ ID NO:17, SEQ ID NO:35, SEQ ID NO:53, SEQ ID NO:71, SEQ ID NO:89, SEQ ID NO:107, SEQ ID NO:125, SEQ ID NO:143, SEQ ID NO:161, SEQ ID NO:179, SEQ ID NO:197, or SEQ ID NO:215.

In some embodiments, the antibody or antigen-binding portion thereof comprises a heavy chain CDR1, CDR2, and CDR3 and a light chain CDR1, CDR2, and CDR3 comprising the sequences of:
(a) SEQ ID NOs: 4, 6, 8, 13, 15, and 17, respectively; or
(b) SEQ ID NOs: 22, 24, 26, 31, 33, and 35, respectively; or
(c) SEQ ID NOs: 40, 42, 44, 49, 51, and 53, respectively; or
(d) SEQ ID NOs: 58, 60, 62, 67, 69, and 71, respectively; or
(e) SEQ ID NOs: 76, 78, 80, 85, 87, and 89, respectively; or
(f) SEQ ID NOs: 94, 96, 98, 103, 105, and 107, respectively; or
(g) SEQ ID NOs: 112, 114, 116, 121, 123, and 125, respectively; or
(h) SEQ ID NOs: 130, 132, 134, 139, 141, and 143, respectively; or
(i) SEQ ID NOs: 148, 150, 152, 157, 159, and 161, respectively; or
(j) SEQ ID NOs: 166, 168, 170, 175, 177, and 179, respectively; or
(k) SEQ ID NOs: 184, 186, 188, 193, 195, and 197, respectively; or
(l) SEQ ID NOs: 202, 204, 206, 211, 213, and 215, respectively; or
(m) SEQ ID NOs: 221, 222, 223, 13, 15, and 17, respectively; or
(n) SEQ ID NOs: 224, 225, 62, 67, 69, and 71, respectively; or
(o) SEQ ID NOs: 226, 227, 228, 67, 69, and 71, respectively; or
(p) SEQ ID NOs: 224, 229, 230, 67, 69, and 71, respectively; or
(q) SEQ ID NOs: 224, 227, 230, 67, 69, and 71, respectively; or
(r) SEQ ID NOs: 231, 232, 235, 103, 105, and 107, respectively; or
(s) SEQ ID NOs: 233, 234, 236, 103, 105, and 107, respectively; or
(t) SEQ ID NOs: 233, 234, 237, 103, 105, and 107, respectively; or
(u) SEQ ID NOs: 166, 238, 170, 175, 177, and 179, respectively; or
(v) SEQ ID NOs: 239, 240, 170, 175, 177, and 179, respectively; or
(w) SEQ ID NOs: 239, 240, 241, 175, 177, and 179, respectively; or
(x) SEQ ID NOs: 239, 240, 242, 175, 177, and 179, respectively; or
(y) SEQ ID NOs: 243, 168, 244, 175, 177, and 179, respectively.

In some embodiments, the antibody or antigen-binding portion thereof comprises:
(a) a heavy chain variable region comprising an amino acid sequence that has at least 90% sequence identity to SEQ ID NO:1, SEQ ID NO:19, SEQ ID NO:37, SEQ ID NO:55, SEQ ID NO:73, SEQ ID NO:91, SEQ ID NO:109, SEQ ID NO:127, SEQ ID NO:145, SEQ ID NO:163, SEQ ID NO:181, SEQ ID NO:199, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, or SEQ ID NO:257; and/or
(b) a light chain variable region comprising an amino acid sequence that has at least 90% sequence identity to SEQ ID NO:10, SEQ ID NO:28, SEQ ID NO:46, SEQ ID NO:64, SEQ ID NO:82, SEQ ID NO:100, SEQ ID NO: 118, SEQ ID NO:136, SEQ ID NO:154, SEQ ID NO:172, SEQ ID NO:190, or SEQ ID NO:208.

In some embodiments, the antibody or antigen-binding portion thereof comprises:
(a) a heavy chain variable region comprising an amino acid sequence that has at least 90% sequence identity to SEQ ID NO:1 or SEQ ID NO:245 and a light chain variable region comprising an amino acid sequence that has at least 90% sequence identity to SEQ ID NO:10; or
(b) a heavy chain variable region comprising an amino acid sequence that has at least 90% sequence identity to SEQ ID NO:19 and a light chain variable region comprising an amino acid sequence that has at least 90% sequence identity to SEQ ID NO:28; or
(c) a heavy chain variable region comprising an amino acid sequence that has at least 90% sequence identity to SEQ ID NO:37 and a light chain variable region comprising an amino acid sequence that has at least 90% sequence identity to SEQ ID NO:46; or
(d) a heavy chain variable region comprising an amino acid sequence that has at least 90% sequence identity to any one of SEQ ID NO:55, SEQ ID NO:246, SEQ ID NO:247, SEQ ID NO:248, or SEQ ID NO:249 and a light chain variable region comprising an amino acid sequence that has at least 90% sequence identity to SEQ ID NO:64; or
(e) a heavy chain variable region comprising an amino acid sequence that has at least 90% sequence identity to SEQ ID NO:73 and a light chain variable region comprising an amino acid sequence that has at least 90% sequence identity to SEQ ID NO:82; or
(f) a heavy chain variable region comprising an amino acid sequence that has at least 90% sequence identity to any one of SEQ ID NO:91, SEQ ID NO:250, SEQ ID NO:251, or SEQ ID NO:252 and a light chain variable region comprising an amino acid sequence that has at least 90% sequence identity to SEQ ID NO:100; or
(g) a heavy chain variable region comprising an amino acid sequence that has at least 90% sequence identity to SEQ ID NO:109 and a light chain variable region comprising an amino acid sequence that has at least 90% sequence identity to SEQ ID NO:118; or
(h) a heavy chain variable region comprising an amino acid sequence that has at least 90% sequence identity to SEQ ID NO:127 and a light chain variable region comprising an amino acid sequence that has at least 90% sequence identity to SEQ ID NO:136; or
(i) a heavy chain variable region comprising an amino acid sequence that has at least 90% sequence identity to SEQ ID NO:145 and a light chain variable region comprising an amino acid sequence that has at least 90% sequence identity to SEQ ID NO:154; or
(j) a heavy chain variable region comprising an amino acid sequence that has at least 90% sequence identity to any one of SEQ ID NO:163, SEQ ID NO:253, SEQ ID NO:254, SEQ ID NO:255, SEQ ID NO:256, or SEQ ID NO:257 and a light chain variable region comprising an amino acid sequence that has at least 90% sequence identity to SEQ ID NO:172; or
(k) a heavy chain variable region comprising an amino acid sequence that has at least 90% sequence identity to SEQ ID NO:181 and a light chain variable region comprising an amino acid sequence that has at least 90% sequence identity to SEQ ID NO:190; or
(l) a heavy chain variable region comprising an amino acid sequence that has at least 90% sequence identity to SEQ ID NO:199 and a light chain variable region comprising an amino acid sequence that has at least 90% sequence identity to SEQ ID NO:208.

In another aspect, antibodies or antigen-binding portions thereof that bind to human TIGIT are provided, wherein the antibody or antigen-binding portion thereof binds to an epitope on human TIGIT that comprises amino acid positions 81 and 82. In some embodiments, the epitope comprises Phe at position 81 and/or Lys or Ser at position 82. In some embodiments, the epitope comprises Phe81 and Lys82.

In some embodiments, the epitope is a discontinuous epitope.

In some embodiments, the antibody or antigen-binding portion thereof binds to an epitope on human TIGIT that further comprises one or more of amino acid positions 51, 52, 53, 54, 55, 73, 74, 75, 76, 77, 79, 83, 84, 85, 86, 87, 88, 89, 90, 91, 92, or 93. In some embodiments, the epitope further comprises one or more amino acid residues selected from the group consisting of Thr51, Ala52, Gln53, Val54, Thr55, Leu73, Gly74, Trp75, His76, Ile77, Pro79, Asp83, Arg84, Val85, Ala86, Pro87, Gly88, Pro89, Gly90, Leu91, Gly92, and Leu93. In some embodiments, the epitope comprises the amino acid residues Thr51, Ala52, Gln53, Val54, Thr55, Gly74, Trp75, His76, Ile77, Phe81, Lys82, Pro87, Gly88, Pro89, Gly90, Leu91, Gly92, and Leu93. In some embodiments, the epitope comprises the amino acid residues Ala52, Gln53, Leu73, Gly74, Trp75, Pro79, Phe81, Lys82, Asp83, Arg84, Val85, and Ala86. In some embodiments, the epitope comprises the sequence ICNADLGWHISPSFK (SEQ ID NO:258).

In still another aspect, antibodies or antigen-binding portions thereof comprising one or more sequences as disclosed herein (e.g., one or more sequences listed in Table 3 below) are provided. In some embodiments, the antibody or antigen-binding portion thereof comprises one more CDR, heavy chain variable region, light chain variable region, or framework region sequences as disclosed herein (e.g., as listed in Table 3 below). In some embodiments, the antibody or antigen-binding portion thereof comprises one or more of:
(a) a heavy chain CDR1 comprising the sequence of any of SEQ ID NO:4, SEQ ID NO:22, SEQ ID NO:40, SEQ ID NO:58, SEQ ID NO:76, SEQ ID NO:94, SEQ ID NO: 112, SEQ ID NO:130, SEQ ID NO:148, SEQ ID NO:166, SEQ ID NO:184, SEQ ID NO:202, SEQ ID NO:221, SEQ ID NO:224, SEQ ID NO:226, SEQ ID NO:231, SEQ ID NO:233, SEQ ID NO:239, or SEQ ID NO:243;
(b) a heavy chain CDR2 comprising the sequence of any of SEQ ID NO:6, SEQ ID NO:24, SEQ ID NO:42, SEQ ID NO:60, SEQ ID NO:78, SEQ ID NO:96, SEQ ID NO:114, SEQ ID NO:132, SEQ ID NO:150, SEQ ID NO:168, SEQ ID NO:186, SEQ ID NO:204, SEQ ID NO:222, SEQ ID NO:225, SEQ ID NO:227, SEQ ID NO:229, SEQ ID NO:232, SEQ ID NO:234, SEQ ID NO:238, or SEQ ID NO:240;
(c) a heavy chain CDR3 comprising the sequence of any of SEQ ID NO:8, SEQ ID NO:26, SEQ ID NO:44, SEQ ID NO:62, SEQ ID NO:80, SEQ ID NO:98, SEQ ID NO:116, SEQ ID NO:134, SEQ ID NO:152, SEQ ID NO:170, SEQ ID NO:188, SEQ ID NO:206, SEQ ID NO:223, SEQ ID NO:228, SEQ ID NO:230, SEQ ID NO:235, SEQ ID NO:236, SEQ ID NO:237, SEQ ID NO:241, SEQ ID NO:242, or SEQ ID NO:244;
(d) a light chain CDR1 comprising the sequence of any of SEQ ID NO:13, SEQ ID NO:31, SEQ ID NO:49, SEQ ID NO:67, SEQ ID NO:85, SEQ ID NO:103, SEQ ID NO:121, SEQ ID NO:139, SEQ ID NO:157, SEQ ID NO:175, SEQ ID NO:193, or SEQ ID NO:211;
(e) a light chain CDR2 comprising the sequence of any of SEQ ID NO:15, SEQ ID NO:33, SEQ ID NO:51, SEQ ID NO:69, SEQ ID NO:87, SEQ ID NO:105, SEQ ID NO: 123, SEQ ID NO:141, SEQ ID NO:159, SEQ ID NO:177, SEQ ID NO:195, or SEQ ID NO:213; or
(f) a light chain CDR3 comprising the sequence of any of SEQ ID NO:17, SEQ ID NO:35, SEQ ID NO:53, SEQ ID NO:71, SEQ ID NO:89, SEQ ID NO:107, SEQ ID NO:125, SEQ ID NO:143, SEQ ID NO:161, SEQ ID NO:179, SEQ ID NO:197, or SEQ ID NO:215.

In some embodiments, the antibody or antigen-binding portion thereof comprises a heavy chain CDR1, CDR2, and CDR3 and a light chain CDR1, CDR2, and CDR3 comprising the sequences of:
(a) SEQ ID NOs: 4, 6, 8, 13, 15, and 17, respectively; or
(b) SEQ ID NOs: 22, 24, 26, 31, 33, and 35, respectively; or
(c) SEQ ID NOs: 40, 42, 44, 49, 51, and 53, respectively; or
(d) SEQ ID NOs: 58, 60, 62, 67, 69, and 71, respectively; or
(e) SEQ ID NOs: 76, 78, 80, 85, 87, and 89, respectively; or
(f) SEQ ID NOs: 94, 96, 98, 103, 105, and 107, respectively; or
(g) SEQ ID NOs: 112, 114, 116, 121, 123, and 125, respectively; or
(h) SEQ ID NOs: 130, 132, 134, 139, 141, and 143, respectively; or
(i) SEQ ID NOs: 148, 150, 152, 157, 159, and 161, respectively; or
(j) SEQ ID NOs: 166, 168, 170, 175, 177, and 179, respectively; or
(k) SEQ ID NOs: 184, 186, 188, 193, 195, and 197, respectively; or
(l) SEQ ID NOs: 202, 204, 206, 211, 213, and 215, respectively; or
(m) SEQ ID NOs: 221, 222, 223, 13, 15, and 17, respectively; or
(n) SEQ ID NOs: 224, 225, 62, 67, 69, and 71, respectively; or
(o) SEQ ID NOs: 226, 227, 228, 67, 69, and 71, respectively; or
(p) SEQ ID NOs: 224, 229, 230, 67, 69, and 71, respectively; or
(q) SEQ ID NOs: 224, 227, 230, 67, 69, and 71, respectively; or
(r) SEQ ID NOs: 231, 232, 235, 103, 105, and 107, respectively; or
(s) SEQ ID NOs: 233, 234, 236, 103, 105, and 107, respectively; or
(t) SEQ ID NOs: 233, 234, 237, 103, 105, and 107, respectively; or
(u) SEQ ID NOs: 166, 238, 170, 175, 177, and 179, respectively; or
(v) SEQ ID NOs: 239, 240, 170, 175, 177, and 179, respectively; or
(w) SEQ ID NOs: 239, 240, 241, 175, 177, and 179, respectively; or
(x) SEQ ID NOs: 239, 240, 242, 175, 177, and 179, respectively; or
(y) SEQ ID NOs: 243, 168, 244, 175, 177, and 179, respectively.

In some embodiments, the antibody or antigen-binding portion thereof comprises:
(a) a heavy chain variable region comprising an amino acid sequence that has at least 90% sequence identity to SEQ ID NO:1, SEQ ID NO:19, SEQ ID NO:37, SEQ ID NO:55, SEQ ID NO:73, SEQ ID NO:91, SEQ ID NO:109, SEQ ID NO:127, SEQ ID NO:145, SEQ ID NO:163, SEQ ID NO:181, SEQ ID NO:199, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, or SEQ ID NO:257; and/or
(b) a light chain variable region comprising an amino acid sequence that has at least 90% sequence identity to SEQ ID NO:10, SEQ ID NO:28, SEQ ID NO:46, SEQ ID NO:64, SEQ ID NO:82, SEQ ID NO:100, SEQ ID NO: 118, SEQ ID NO:136, SEQ ID NO:154, SEQ ID NO:172, SEQ ID NO:190, or SEQ ID NO:208.

In some embodiments, the antibody or antigen-binding portion thereof comprises:
(a) a heavy chain variable region comprising an amino acid sequence that has at least 90% sequence identity to SEQ ID NO:1 or SEQ ID NO:245 and a light chain variable region comprising an amino acid sequence that has at least 90% sequence identity to SEQ ID NO:10; or
(b) a heavy chain variable region comprising an amino acid sequence that has at least 90% sequence identity to SEQ ID NO:19 and a light chain variable region comprising an amino acid sequence that has at least 90% sequence identity to SEQ ID NO:28; or
(c) a heavy chain variable region comprising an amino acid sequence that has at least 90% sequence identity to SEQ ID NO:37 and a light chain variable region comprising an amino acid sequence that has at least 90% sequence identity to SEQ ID NO:46; or
(d) a heavy chain variable region comprising an amino acid sequence that has at least 90% sequence identity to any one of SEQ ID NO:55, SEQ ID NO:246, SEQ ID NO:247, SEQ ID NO:248, or SEQ ID NO:249 and a light chain variable region comprising an amino acid sequence that has at least 90% sequence identity to SEQ ID NO:64; or
(e) a heavy chain variable region comprising an amino acid sequence that has at least 90% sequence identity to SEQ ID NO:73 and a light chain variable region comprising an amino acid sequence that has at least 90% sequence identity to SEQ ID NO:82; or
(f) a heavy chain variable region comprising an amino acid sequence that has at least 90% sequence identity to any one of SEQ ID NO:91, SEQ ID NO:250, SEQ ID NO:251, or SEQ ID NO:252 and a light chain variable region comprising an amino acid sequence that has at least 90% sequence identity to SEQ ID NO:100; or
(g) a heavy chain variable region comprising an amino acid sequence that has at least 90% sequence identity to SEQ ID NO:109 and a light chain variable region comprising an amino acid sequence that has at least 90% sequence identity to SEQ ID NO:118; or
(h) a heavy chain variable region comprising an amino acid sequence that has at least 90% sequence identity to SEQ ID NO:127 and a light chain variable region comprising an amino acid sequence that has at least 90% sequence identity to SEQ ID NO:136; or
(i) a heavy chain variable region comprising an amino acid sequence that has at least 90% sequence identity to SEQ ID NO:145 and a light chain variable region comprising an amino acid sequence that has at least 90% sequence identity to SEQ ID NO:154; or
(j) a heavy chain variable region comprising an amino acid sequence that has at least 90% sequence identity to any one of SEQ ID NO:163, SEQ ID NO:253, SEQ ID NO:254, SEQ ID NO:255, SEQ ID NO:256, or SEQ ID NO:257 and a light chain variable region comprising an amino acid sequence that has at least 90% sequence identity to SEQ ID NO:172; or
(k) a heavy chain variable region comprising an amino acid sequence that has at least 90% sequence identity to SEQ ID NO:181 and a light chain variable region comprising an amino acid sequence that has at least 90% sequence identity to SEQ ID NO:190; or
(l) a heavy chain variable region comprising an amino acid sequence that has at least 90% sequence identity to SEQ ID NO:199 and a light chain variable region comprising an amino acid sequence that has at least 90% sequence identity to SEQ ID NO:208.

In some embodiments, an antibody or antigen-binding portion thereof as disclosed herein exhibits synergy with an anti-PD1 antibody or an anti-PD-L1 antibody.

In some embodiments, an antibody or antigen-binding portion thereof as disclosed herein is a monoclonal antibody. In some embodiments, the antibody is a humanized antibody. In some embodiments, the antibody is a fully human antibody. In some embodiments, the antibody is a chimeric antibody. In some embodiments, the antigen-binding fragment is a Fab, a F(ab′)₂, a scFv, or a bivalent scFv.

In another aspect, pharmaceutical compositions comprising an isolated antibody or antigen-binding portion thereof as described herein and a pharmaceutically acceptable carrier are provided.

In yet another aspect, bispecific antibodies comprising an isolated antibody or antigen-binding portion thereof as described herein are provided.

In yet another aspect, antibody-drug conjugates comprising an isolated antibody or antigen-binding portion thereof as described herein are provided.

In still another aspect, isolated polynucleotides are provided. In some embodiments, the polynucleotide comprises one or more nucleotide sequences encoding an antibody or antigen-binding portion thereof as described herein. In some embodiments, the polynucleotide comprises one or more nucleotide sequences encoding a polypeptide disclosed in Table 3 below. In some embodiments, the polynucleotide comprises one or more nucleotide sequences encoding an antibody, or an antigen-binding portion thereof, that binds to human TIGIT, wherein the isolated polynucleotide comprises:
(a) the nucleotide sequence of SEQ ID NO:2, SEQ ID NO:20, SEQ ID NO:38, SEQ ID NO:56, SEQ ID NO:74, SEQ ID NO:92, SEQ ID NO: 110, SEQ ID NO:128, SEQ ID NO:146, SEQ ID NO:164, SEQ ID NO:182, or SEQ ID NO:200; and/or
(b) the nucleotide sequence of SEQ ID NO:11, SEQ ID NO:29, SEQ ID NO:47, SEQ ID NO:65, SEQ ID NO:83, SEQ ID NO:101, SEQ ID NO:119, SEQ ID NO:137, SEQ ID NO:155, SEQ ID NO:173, SEQ ID NO:191, or SEQ ID NO:209.

In yet another aspect, vectors and host cells comprising a polynucleotide as described herein are provided. In another aspect, methods of producing an antibody comprising culturing a host cell as described herein under conditions suitable for producing the antibody are provided.

In another aspect, kits (e.g., for use in a therapeutic method as described herein) are provided. In some embodiments, the kit comprises an isolated anti-TIGIT antibody or antigen-binding portion thereof as described herein, or a pharmaceutical composition comprising an anti-TIGIT antibody or antigen-binding portion thereof as described herein; and further comprises an immuno-oncology agent. In some embodiments, the immuno-oncology agent is a PD-1 pathway inhibitor. In some embodiments, the PD-1 pathway inhibitor is an anti-PD1 antibody or an anti-PD-L1 antibody. In some embodiments, the PD-1 pathway inhibitor is an antagonist or inhibitor of a T cell coinhibitor. In some embodiments, the immuno-oncology agent is an agonist of a T cell coactivator. In some embodiments, the immuno-oncology agent is an immune stimulatory cytokine.

In another aspect, methods of treating a cancer in a subject are provided. In some embodiments, the method comprises administering to the subject a therapeutic amount of an isolated antibody or antigen-binding portion thereof as described herein, or a pharmaceutical composition as described herein, a bispecific antibody as described herein, or an antibody-drug conjugate as described herein.

In some embodiments, the cancer is a cancer that is enriched for expression of CD112 or CD115. In some embodiments, the cancer is a cancer that is enriched for T cells or natural killer (NK) cells that express TIGIT. In some embodiments, the cancer is bladder cancer, breast cancer, uterine cancer, cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, or sarcoma. In some embodiments, the cancer is a lymphoma or a leukemia.

In some embodiments, the method further comprises administering to the subject a therapeutic amount of an immuno-oncology agent. In some embodiments, the immuno-oncology agent is a PD-1 pathway inhibitor. In some embodiments, the PD-1 pathway inhibitor is an anti-PD1 antibody or an anti-PD-L1 antibody. In some embodiments, the PD-1 pathway inhibitor is an antagonist or inhibitor of a T cell coinhibitor. In some embodiments, the immuno-oncology agent is an agonist of a T cell coactivator. In some embodiments, the immuno-oncology agent is an immune stimulatory cytokine. In some embodiments, the isolated antibody, the pharmaceutical composition, the bispecific antibody, or the antibody-drug conjugate is administered concurrently with the immuno-oncology agent. In some embodiments, the isolated antibody, the pharmaceutical composition, the bispecific antibody, or the antibody-drug conjugate is administered sequentially to the immuno-oncology agent.

### BRIEF DESCRIPTION OF THE DRAWINGS

FIG. 1. Binding of 65 anti-TIGIT antibody clones and an irrelevant isotype control antibody to HEK 293 cells engineered to express human TIGIT (top panel), cynomolgus monkey TIGIT (middle panel), and mouse TIGIT (bottom panel).

FIG. 2. Binding of 65 anti-TIGIT antibody clones and an irrelevant isotype control antibody to primary human T cells (top panel), cynomolgus monkey T cells (middle panel), and mouse T cells (bottom panel). For the bottom panel, 35 of 65 clones were evaluated. Of the 35 clones evaluated, 5 of the 35 did not bind mTIGIT-Fc protein (clones 20, 27, 55, 56, and 60), as indicated by the light green bars.

FIG. 3A-3D. (A-C) Binding titration values of eight anti-TIGIT antibody clones (clones 2, 5, 13, 16, 17, 20, 25, and 54) to human (A), mouse (B), and cynomolgus monkey (C) TIGIT expressed on HEK 293 cells. Results are shown for singlicate wells. (D) EC50 values of eight anti-TIGIT antibody clones (clones 2, 5, 13, 16, 17, 20, 25, and 54) to human, mouse, and cynomolgus monkey TIGIT expressed on HEK 293 cells.

FIG. 4. Binding titration of anti-TIGIT antibody clones 13 and 25 to activated mouse splenic T cells. Results are shown for singlicate wells. Clone 13 had an EC50 of 0.24 μg/mL. Clone 25 had an EC50 of 2.28 μg/mL.

FIG. 5A-5B. Anti-TIGIT antibodies blocked CD155 interaction with TIGIT expressed on HEK 293 cells, for both human CD155 binding to HEK 293 cells expressing human TIGIT (A) and mouse CD155 binding to HEK 293 cells expressing mouse TIGIT (B). Results are shown for singlicate wells.

FIG. 6. Anti-TIGIT antibodies blocked human CD112 interaction with human TIGIT expressed on HEK 293 cells. Results are shown for singlicate wells.

FIG. 7A-7B. (A) Upper panel: Select anti-TIGIT antibodies effectively blocked TIGIT-CD155 engagement, resulting in T cell activation, as measured by a >1.5-fold induction in luciferase activity. About 12 clones showed >1.5-fold induction in the bioassay. Two clones did not block TIGIT-CD155 interaction in ForteBio assay (pink bars). Fold induction was measured over no Ab control. Mean and SD are of duplicate experiments; antibodies were at 20 μg/mL. Gray bar=hIgG1 isotype control. Black bar=no antibody control (defined as baseline). Lower panel: Correlation plot of TIGIT/CD155 blockade bioassay versus TIGIT-Fc affinity. The activity in the bioassay correlated with affinity for recombinant protein. (B) Dose response of 12 selected anti-TIGIT clones in TIGIT/CD155 blockade bioassay. Clones 13 and 25, which showed strong binding to all three species, showed good activity in the bioassay. Mean and SD are of triplicate wells.

FIG. 8. Select anti-TIGIT antibodies synergized with anti-PD-1, resulting in T cell activation. Mean and SD are of triplicate wells. Both clone 13 and clone 25 showed synergy with anti-PD-1 in combination bioassay.

FIG. 9A-9H. (A-D) Binding titration (A-C) and EC50 values (D) for binding to human (A), mouse (B), and cynomolgus monkey (C) TIGIT expressed on HEK 293 cells for fully human anti-TIGIT clone 13 (“c13 hIgG1”) and mouse IgG1 (“c13 mIgG1”) and mouse IgG2a (“c13 mIgG2a”) chimeras of clone 13. Mean and SD are of duplicate wells. (E-F) Antibodies c13 hIgG1, c13 mIgG1, and c13 mIgG2a blocked CD155 interaction with TIGIT expressed on HEK 293 cells, for both human CD155 binding to HEK 293 cells expressing human TIGIT (E) and mouse CD155 binding to HEK 293 cells expressing mouse TIGIT (F). Results are for singlicate wells. (G) Antibodies c13 hIgG1, c13 mIgG1, and c13 mIgG2a blocked human CD112 interaction with human TIGIT expressed on HEK 293 cells. Results are for singlicate wells. (H) Dose response of parental and chimeric anti-TIGIT antibody clones c1313 hIgG1, c13 mIgG1, and c13 mIgG2a in TIGIT/CD155 blockade bioassay. Mean and SD are of triplicate wells.

FIG. 10A-10K. Anti-TIGIT antibodies that can engage activating Fcgamma receptors mediated anti-tumor efficacy in a CT26 syngeneic tumor model in mice. (A) Group mean tumor volume. (B-K) Individual animal tumor volume for groups 1 through 10. PR=Partial Response (tumor volume is 50% or less of its day 1 volume for three consecutive measurements and equal to or greater than 13.5 mm³ for one or more of these three measurements). CR=Complete Response (tumor volume is less than 13.5 mm³ for three consecutive measurements).

### DETAILED DESCRIPTION OF THE INVENTION

### I. Introduction

As described herein, antibodies having high affinity for human TIGIT (T-cell immunoreceptor with Ig and ITIM domains), and further having cross-reactivity with either or both of mouse TIGIT and cynomolgus monkey TIGIT, have been identified that inhibit the interaction between TIGIT and CD155. These antibodies also exhibit synergy with anti-PD-1 antibodies. Thus, the anti-TIGIT antibodies described herein may be used in a number of therapeutic applications, such as for the treatment of various cancers, either as a single agent or in combination with another therapeutic agent such as anti-PD-1 agents or anti-PD-L1 agents.

Accordingly, in one aspect, the present invention provides compositions, kits, and methods of treatment comprising an antibody or antigen-binding portion of an antibody, that binds to human TIGIT.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the singular forms “a”, “an” and “the” include plural referents unless the content clearly dictates otherwise. Thus, for example, reference to “an antibody” optionally includes a combination of two or more such molecules, and the like.

The term “about,” as used herein, refers to the usual error range for the respective value readily known to the skilled person in this technical field.

As used herein, the term “TIGIT” refers to “T-cell immunoreceptor with Ig and ITIM domains.” The protein encoded by the TIGIT gene is a member of the CD28 family within the Ig superfamily of proteins. TIGIT is expressed on several classes of T cells and on natural killer (NK) cells and mediates its immunosuppressive effect by competing with CD226 for the ligands CD155 and CD112. See, Levin et al., Eur. J. Immunol., 2011, 41:902-915. TIGIT is also referred to in the art as WUCAM (Washington University Cell Adhesion Molecule) and VSTM3 (HUGO designation). See, Levin et al., Eur J Immunol, 2011, 41:902-915. Accordingly, reference to “TIGIT” throughout this application also includes a reference to WUCAM and/or VSTM3 unless otherwise stated or apparent from context. Human TIGIT nucleotide and protein sequences are set forth in, e.g., Genbank Accession Nos. NM173799 (SEQ ID NO:217) and NP776160 (SEQ ID NO:218), respectively.

The term “cancer” refers to a disease characterized by the uncontrolled growth of aberrant cells. The term includes all known cancers and neoplastic conditions, whether characterized as malignant, benign, soft tissue, or solid, and cancers of all stages and grades including pre- and post-metastatic cancers. Examples of different types of cancer include, but are not limited to, digestive and gastrointestinal cancers such as gastric cancer (e.g., stomach cancer), colorectal cancer, gastrointestinal stromal tumors, gastrointestinal carcinoid tumors, colon cancer, rectal cancer, anal cancer, bile duct cancer, small intestine cancer, and esophageal cancer; breast cancer; lung cancer; gallbladder cancer; liver cancer; pancreatic cancer; appendix cancer; prostate cancer, ovarian cancer; renal cancer; cancer of the central nervous system; skin cancer (e.g., melanoma); lymphomas; gliomas; choriocarcinomas; head and neck cancers; osteogenic sarcomas; and blood cancers. As used herein, a “tumor” comprises one or more cancerous cells.

The term “antibody” refers to a polypeptide encoded by an immunoglobulin gene or functional fragments thereof that specifically binds and recognizes an antigen (e.g., human TIGIT), a particular cell surface marker, or any desired target. Typically, the “variable region” contains the antigen-binding region of the antibody (or its functional equivalent) and is most critical in specificity and affinity of binding. See, Fundamental Immunology 7^{th} Edition, Paul, ed., Wolters Kluwer Health/Lippincott Williams & Wilkins (2013). The recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

An “isotype” is a class of antibodies defined by the heavy chain constant region. Immunoglobulin genes include the kappa, lambda, alpha, gamma, delta, epsilon, and mu constant region genes. Light chains are classified as either kappa or lambda. Heavy chains are classified as gamma, mu, alpha, delta, or epsilon, which in turn define the isotype classes, IgG, IgM, IgA, IgD and IgE, respectively.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson and Wu, Nucleic Acids Res. 2000 Jan. 1; 28(1): 214-218 and Johnson et al., Nucleic Acids Res., 29:205-206 (2001); Chothia & Lesk, (1987) J Mol. Biol. 196, 901-917; Chothia et al. (1989) Nature 342, 877-883; Chothia et al. (1992) J. Mol. Biol. 227, 799-817; Al-Lazikani et al., J. Mol. Biol 1997, 273(4)). Unless otherwise indicated, CDRs are determined according to Kabat. Definitions of antigen combining sites are also described in the following: Ruiz et al. Nucleic Acids Res., 28, 219-221 (2000); and Lefranc Nucleic Acids Res. January 1; 29(1):207-9 (2001); MacCallum et al., J. Mol. Biol., 262: 732-745 (1996); and Martin et al, Proc. Natl Acad. Sci. USA, 86, 9268-9272 (1989); Martin, et al, Methods Enzymol., 203: 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

The terms “antigen-binding portion” or “antigen-binding fragment” are used interchangeably herein and refer to one or more fragments of an antibody that retain the ability to specifically bind to an antigen (e.g., TIGIT). It has been shown that the antigen-binding function of an antibody can be performed by fragments of a full-length antibody. Examples of antigen binding fragments include, but are not limited to, a Fab fragment (a monovalent fragment consisting of the VL, VH, CL and CHi domains), a F(ab′)₂ fragment (a bivalent fragment comprising two Fab fragments linked by a disulfide bridge at the hinge region), single chain Fv (scFv), complementarity determining regions (CDRs), VL (light chain variable region), VH (heavy chain variable region), disulfide-linked Fvs (dsFv), and any combination of those or any other functional portion of an immunoglobulin peptide capable of binding to target antigen (see, e.g., Fundamental Immunology, supra). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries and yeast-based antibody library presentation systems (see, e.g., McCafferty et al., (1990) Nature 348:552; Y. Xu et al., PEDS, 2013, 26:663-670; WO 2009/036379; WO 2010/105256; and WO 2012/009568). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A “monoclonal antibody” refers to a clonal preparation of antibodies with a single binding specificity and affinity for a given epitope on an antigen. A “polyclonal antibody” refers to a preparation of antibodies that are raised against a single antigen, but with different binding specificities and affinities.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

As used herein, the term “chimeric antibody” refers to an antibody molecule in which (a) the constant region, or a portion thereof, is altered, replaced or exchanged so that the antigen binding site (variable region, CDR, or portion thereof) is linked to a constant region of a different or altered class, effector function and/or species, or an entirely different molecule which confers new properties to the chimeric antibody (e.g., an enzyme, toxin, hormone, growth factor, drug, etc.); or (b) the variable region, or a portion thereof, is altered, replaced or exchanged with a variable region having a different or altered antigen specificity (e.g., CDR and framework regions from different species).

The term “epitope” refers to the area or region of an antigen to which an antibody specifically binds, i.e., an area or region in physical contact with the antibody, and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

The phrase “specifically binds” refers to a molecule (e.g., antibody or antibody fragment) that binds to a target with greater affinity, avidity, more readily, and/or with greater duration to that target in a sample than it binds to a non-target compound. In some embodiments, an antibody or antigen-binding portion thereof that specifically binds a target is an antibody or antigen-binding portion that binds to the target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds TIGIT will typically bind to TIGIT with at least a 2-fold greater affinity than to a non-TIGIT target. It will be understood by a person of ordinary skill in the art reading this definition, for example, that an antibody (or moiety or epitope) that specifically or preferentially binds to a first target may or may not specifically or preferentially bind to a second target.

The term “binding affinity” is herein used as a measure of the strength of a non-covalent interaction between two molecules, e.g., an antibody, or fragment thereof, and an antigen. The term “binding affinity” is used to describe monovalent interactions (intrinsic activity).

Binding affinity between two molecules, e.g. an antibody, or fragment thereof, and an antigen, through a monovalent interaction may be quantified by determination of the dissociation constant (K_{D}). In turn, K_{D} can be determined by measurement of the kinetics of complex formation and dissociation using, e.g., the surface plasmon resonance (SPR) method (Biacore™). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those, for example, set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of ligands such as antibodies towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art or as described in the Examples section below, such as Surface Plasmon Resonance (SPR), e.g. by using a Biacore™ system; kinetic exclusion assays such as KinExA®; and BioLayer interferometry (e.g., using the ForteBio® Octet platform). In some embodiments, binding affinity is determined using a BioLayer interferometry assay. See, e.g., Wilson et al., Biochemistry and Molecular Biology Education, 38:400-407 (2010); Dysinger et al., J Immunol. Methods, 379:30-41 (2012); and Estep et al., Mabs, 2013, 5:270-278.

The term “cross-reacts,” as used herein, refers to the ability of an antibody to bind to an antigen other than the antigen against which the antibody was raised. In some embodiments, cross-reactivity refers to the ability of an antibody to bind to an antigen from another species than the antigen against which the antibody was raised. As a non-limiting example, an anti-TIGIT antibody as described herein that is raised against a human TIGIT antigen can exhibit cross-reactivity with TIGIT from a different species (e.g., mouse or monkey).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “nucleic acid” and “polynucleotide” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single and double stranded DNA, single and double stranded RNA, and hybrid molecules having mixtures of single and double stranded DNA and RNA.

The term “isolated,” as used with reference to a nucleic acid or protein (e.g., antibody), denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode proteins other than the protein encoded by the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “immuno-oncology agent” refers to an agent that enhances, stimulates, or upregulates an immune response against a cancer in a subject (e.g., in stimulating an immune response for inhibiting tumor growth). In some embodiments, an immuno-oncology agent is a small molecule, antibody, peptide, protein, circular peptide, peptidomimetic, polynucleotide, inhibitory RNA, aptamer, drug compound, or other compound. In some embodiments, an immuno-oncology agent is an antagonist or inhibitor of PD-1 or the PD-1 pathway.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

The terms “therapy,” “treatment,” and “amelioration” refer to any reduction in the severity of symptoms. In the case of treating cancer, treatment can refer to reducing, e.g., tumor size, number of cancer cells, growth rate, metastatic activity, cell death of non-cancer cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (no detectable symptoms remaining) or partial, such that symptoms are less frequent or severe than in a patient without the treatment described herein. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

As used herein, a “therapeutic amount” or “therapeutically effective amount” of an agent (e.g., an antibody as described herein) is an amount of the agent that prevents, alleviates, abates, or reduces the severity of symptoms of a disease (e.g., a cancer) in a subject. For example, for the given parameter, a therapeutically effective amount will show an increase or decrease of therapeutic effect of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The terms “administer,” “administered,” or “administering” refer to methods of delivering agents, compounds, or compositions to the desired site of biological action. These methods include, but are not limited to, topical delivery, parenteral delivery, intravenous delivery, intradermal delivery, intramuscular delivery, colonical delivery, rectal delivery, or intraperitoneal delivery. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

### III. Antibodies Against TIGIT

In one aspect, antibodies and antigen-binding portions of antibodies that bind to human TIGIT (T-cell immunoreceptor with Ig and ITIM domains) are provided. As described herein, in some embodiments, the anti-TIGIT antibody inhibits interaction between TIGIT and one or both of the ligands CD155 and CD112. In some embodiments, the anti-TIGIT antibody inhibits the interaction between TIGIT and CD155 in a functional bioassay, allowing CD155-CD226 signaling to occur. In some embodiments, the anti-TIGIT antibody exhibits synergy with an anti-PD-1 agent (e.g., an anti-PD-1 antibody) or an anti-PD-L1 agent (e.g., an anti-PD-L1 antibody).

### Characteristics of Anti-TIGIT Antibodies

In some embodiments, an anti-TIGIT antibody binds to human TIGIT protein (SEQ ID NO:218) or a portion thereof with high affinity. In some embodiments, the antibody has a binding affinity (K_{D}) for human TIGIT of less than 5 nM, less than 1 nM, less than 500 pM, less than 250 pM, less than 150 pM, less than 100 pM, less than 50 pM, less than 40 pM, less than 30 pM, less than 20 pM, or less than about 10 pM. In some embodiments, the antibody has a binding affinity (K_{D}) for human TIGIT of less than 50 pM. In some embodiments, the antibody has a K_{D} for human TIGIT in the range of about 1 pM to about 5 nM, e.g., about 1 pM to about 1 nM, about 1 pM to about 500 pM, about 5 pM to about 250 pM, or about 10 pM to about 100 pM.

In some embodiments, in addition to binding to human TIGIT with high affinity, the anti-TIGIT antibody exhibits cross-reactivity with cynomolgus monkey (“cyno”) TIGIT (e.g., a cyno TIGIT protein having the sequence of SEQ ID NO:219) and/or mouse TIGIT (e.g., a mouse TIGIT protein having the sequence of SEQ ID NO:220). In some embodiments, the anti-TIGIT antibody binds to mouse TIGIT (e.g., a mouse TIGIT having the sequence of SEQ ID NO:220) with a binding affinity (K_{D}) of 100 nM or less. In some embodiments, the anti-TIGIT antibody binds to human TIGIT with a K_{D} of 5 nM or less, and cross-reacts with mouse TIGIT with a K_{D} of 100 nM or less. In some embodiments, an anti-TIGIT antibody that binds to a human TIGIT also exhibits cross-reactivity with both cynomolgus monkey TIGIT and mouse TIGIT.

In some embodiments, antibody cross-reactivity is determined by detecting specific binding of the anti-TIGIT antibody to TIGIT that is expressed on a cell (e.g., a cell line that expresses human TIGIT, cyno TIGIT, or mouse TIGIT, or a primary cell that endogenously expresses TIGIT, e.g., primary T cells that endogenously express human TIGIT, cyno TIGIT, or mouse TIGIT). In some embodiments, antibody binding and antibody cross-reactivity is determined by detecting specific binding of the anti-TIGIT antibody to purified or recombinant TIGIT (e.g., purified or recombinant human TIGIT, purified or recombinant cyno TIGIT, or purified or recombinant mouse TIGIT) or a chimeric protein comprising TIGIT (e.g., an Fc-fusion protein comprising human TIGIT, cyno TIGIT, or mouse TIGIT, or a His-tagged protein comprising human TIGIT, cyno TIGIT, or mouse TIGIT).

Methods for analyzing binding affinity, binding kinetics, and cross-reactivity are known in the art. See, e.g., Ernst et al., Determination of Equilibrium Dissociation Constants, Therapeutic Monoclonal Antibodies (Wiley & Sons ed. 2009). These methods include, but are not limited to, solid-phase binding assays (e.g., ELISA assay), immunoprecipitation, surface plasmon resonance (SPR, e.g., Biacore™ (GE Healthcare, Piscataway, N.J.)), kinetic exclusion assays (e.g. KinExA®), flow cytometry, fluorescence-activated cell sorting (FACS), BioLayer interferometry (e.g., Octet™ (FortéBio, Inc., Menlo Park, Calif.)), and Western blot analysis. SPR techniques are reviewed, e.g., in Hahnfeld et al. Determination of Kinetic Data Using SPR Biosensors, Molecular Diagnosis ofInfectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given wavelength is shined on the surface, the changes to the optical reflectivity of the gold indicate binding, and the kinetics of binding. In some embodiments, kinetic exclusion assays are used to determine affinity. This technique is described, e.g., in Darling et al., Assay and Drug Development Technologies Vol. 2, number 6 647-657 (2004). In some embodiments, BioLayer interferometry assays are used to determine affinity. This technique is described, e.g., in Wilson et al., Biochemistry and Molecular Biology Education, 38:400-407 (2010); Dysinger et al., J. Immunol. Methods, 379:30-41 (2012).

In some embodiments, the anti-TIGIT antibodies and antigen-binding portions thereof of the instant disclosure inhibit interaction between TIGIT and the ligand CD155. In some embodiments, the anti-TIGIT antibodies and antigen-binding portions thereof inhibit interaction between TIGIT and the ligand CD112. In some embodiments, the anti-TIGIT antibodies and antigen-binding portions thereof inhibit interaction between TIGIT and both of the ligands CD155 and CD112.

In some embodiments, the ability of an anti-TIGIT antibody to inhibit interactions between TIGIT and CD155 and/or CD112 is evaluated by measuring whether physical interactions between TIGIT and CD155 or CD112 decrease in a binding assay. In some embodiments, the binding assay is a competitive binding assay. The assay may be performed in various formats, such as but not limited to an ELISA assay, flow cytometry, a surface plasmon resonance (SPR) assay (e.g., Biacore™), or BioLayer interferometry (e.g., ForteBio Octet™). See, e.g., Duff et al., Biochem J., 2009, 419:577-584; Dysinger et al., J. Immunol. Methods, 379:30-41 (2012); and Estep et al, Mabs, 2013, 5:270-278.

In some embodiments, the anti-TIGIT antibody inhibits the interaction between TIGIT and CD155 in a functional bioassay, such as a functional cellular assay in which inhibition of TIGIT/CD155 interaction is evaluated by measuring activation of CD155-CD226 signaling in the cell (e.g., via activation of a downstream reporter). A non-limiting exemplary functional cellular assay is described in the Examples section below. In this exemplary functional assay, luciferase expression requires TCR engagement and a co-stimulatory signal from CD155-CD226. A first cell (also referred to as a “T effector cell”) expresses a TCR complex, TIGIT, and CD226 on the cell surface and contains a luciferase gene. A second cell (also referred to as an “artificial antigen presenting cell”) expresses a TCR activator and CD155. Co-culture of the cells in the absence of anti-TIGIT antibody results in a TIGIT-CD155 interaction that inhibits co-stimulation of the effector cell by CD155-CD226, preventing expression of luciferase by the effector cell. In the presence of an anti-TIGIT antibody that inhibits the interaction between TIGIT and CD155, CD155 and CD226 are able to interact and produce a co-stimulatory signal that drives luciferase expression in the first cell. Such functional cellular assays are described in the art, e.g., Cong et al., Genetic Engineering and Biotechnology News, 2015, 35(10):16-17, and are also commercially available (e.g., TIGIT/CD155 Blockade Bioassay Kit, Promega Corp., Madison, Wis.). In some embodiments, an anti-TIGIT antibody that inhibits the interaction between TIGIT and CD155 increases the level or amount of activation of CD155-CD226 signaling (e.g., as measured in a cellular assay such as the TIGIT/CD155 Blockade Bioassay Kit) by at least 10%, at least 20%, at least 30%, at least 40%, at least 50%, at least 60%, at least 70%, at least 80%, at least 90%, or more as compared to the level or amount of CD155-CD226 signaling in the absence of the anti-TIGIT antibody. In some embodiments, an anti-TIGIT antibody that inhibits the interaction between TIGIT and CD155 increases the level or amount of activation of CD155-CD226 signaling (e.g., as measured in a cellular assay such as the TIGIT/CD155 Blockade Bioassay Kit) by at least about 1.2-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold or more as compared to the level or amount of CD155-CD226 signaling in the absence of the anti-TIGIT antibody.

In some embodiments, an anti-TIGIT antibody that binds to human TIGIT (and optionally exhibits cross-reactivity with cynomolgus monkey and/or mouse TIGIT and/or optionally inhibits interaction between TIGIT and CD155 and/or CD112) exhibits synergy with an anti-PD-1 agent (e.g., an anti-PD-1 antibody). In some embodiments, the anti-TIGIT antibody enhances the effect of the anti-PD-1 agent (e.g., anti-PD-1 antibody) by at least about 1.2-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold or more.

In some embodiments, the anti-TIGIT antibody exhibits synergy with an anti-PD-1 agent (e.g., an anti-PD-1 antibody) in a functional bioassay, such as a functional cellular assay in which inhibition of TIGIT signaling and inhibition of PD-1 signaling is evaluated by measuring the activation of signaling in an effector cell. A non-limiting exemplary functional cellular assay is described in the Examples section below. In this exemplary functional assay, a first cell (also referred to as a “T effector cell”) expresses a TCR complex, TIGIT, CD226, and PD-1 on the cell surface and contains a luciferase gene. A second cell (also referred to as an “artificial antigen presenting cell”) expresses a TCR activator, CD155, and PD-L1. Expression of the luciferase gene by the effector cell is activated by either or both of (1) blockade of TIGIT-CD155 interaction, thereby allowing CD155-CD226 interaction and subsequent co-stimulation of luciferase expression by the effector cell, or (2) blockade of PD-1/PD-L1 interaction, thereby relieving the inhibition of luciferase expression by the effector cell. The level of luciferase expression in the absence or presence of anti-TIGIT antibodies and anti-PD-1 agents or anti-PD-L1 agents can be measured and quantified for determining whether an anti-TIGIT antibody exhibits synergy with the anti-PD-1 agent or the anti-PD-L1 agent. Such functional cellular assays are described in the art (e.g., Cong et al., Genetic Engineering and Biotechnology News, 2015, 35(10):16-17), and are also commercially available (e.g., PD-1/TIGIT Combination Bioassay Kit, Promega Corp., Madison, Wis.).

In some embodiments, the efficacy of an anti-TIGIT antibody, as well as whether the anti-TIGIT antibody inhibits synergistically with an anti-PD-1 agent (e.g., an anti-PD-1 antibody) or an anti-PD-L1 agent (e.g., an anti-PD-L1 antibody), can be measured using an in vivo model, e.g., an in vivo tumor model. For example, the efficacy of an anti-TIGIT antibody as described herein, or the efficacy of an anti-TIGIT antibody as described herein when administered in combination with an anti-PD-1 agent or an anti-PD-L1 agent can be evaluated using a syngeneic mouse tumor model. Suitable syngeneic tumor models are described in the art. See, e.g., Rios-Doria et al., Neoplasia, 2015, 17:661-670; and Moynihan et al., Nature Medicine, 2016, doi:10.1038/nm.4200. In some embodiments, an anti-TIGIT antibody reduces the size of a tumor or the overall number of tumors in an in vivo model by at least 10%, at least 20%, at least 30%, at least 40%, at least 50%, at least 60%, at least 70%, at least 80%, at least 90% or more as compared to a control or reference value (e.g., as compared to tumor size or overall number of tumors in an untreated control).

In some embodiments, an anti-TIGIT antibody recognizes an epitope of human TIGIT that comprises one or both of amino acid positions 81 and 82, as numbered with reference to SEQ ID NO:218. In some embodiments, an anti-TIGIT antibody recognizes an epitope that comprises Phe at position 81. In some embodiments, an anti-TIGIT antibody recognizes an epitope that comprises Lys or Ser at position 82. In some embodiments, an anti-TIGIT antibody recognizes an epitope that comprises Phe at position 81 and Lys at position 82. In some embodiments, an anti-TIGIT antibody recognizes an epitope that comprises Phe at position 81 and Ser at position 82.

In some embodiments, an anti-TIGIT antibody recognizes a linear epitope that comprises one or both of amino acid positions 81 and 82 (e.g., a discontinuous epitope that comprises Phe at position 81 and Lys or Ser at position 82). In some embodiments, an anti-TIGIT antibody recognizes a discontinuous epitope that comprises one or both of amino acid positions 81 and 82 (e.g., a discontinuous epitope that comprises Phe at position 81 and Lys or Ser at position 82).

In some embodiments, an anti-TIGIT antibody binds to an epitope on human TIGIT that further comprises one or more (e.g., 1, 2, 3, 4, 5, 6, 7, 8, 9, 10, 12, 13, 14, 15 or more) of amino acid positions 51, 52, 53, 54, 55, 73, 74, 75, 76, 77, 79, 83, 84, 85, 86, 87, 88, 89, 90, 91, 92, or 93. In some embodiments, an anti-TIGIT antibody binds to an epitope on human TIGIT that further comprises one or more (e.g., 1, 2, 3, 4, 5, 6, 7, 8, 9, 10, 12, 13, 14, 15 or more) of the following: Thr at position 51, Ala at position 52, Glu or Gln at position 53, Val at position 54, Thr at position 55, Leu at position 73, Gly at position 74, Trp at position 75, His at position 76, Val or Ile at position 77, Ser or Pro at position 79, Asp at position 83, Arg at position 84, Val at position 85, Val or Ala at position 86, Pro at position 87, Gly at position 88, Pro at position 89, Ser or Gly at position 90, Leu at position 91, Gly at position 92, or Leu at position 93. In some embodiments, an anti-TIGIT antibody binds to an epitope on human TIGIT that further comprises one or more (e.g., 1, 2, 3, 4, 5, 6, 7, 8, 9, 10, 12, 13, 14, 15 or more) of the amino acid residues Thr51, Ala52, Gln53, Val54, Thr55, Leu73, Gly74, Trp75, His76, Ile77, Pro79, Asp83, Arg84, Val85, Ala86, Pro87, Gly88, Pro89, Gly90, Leu91, Gly92, and Leu93.

In some embodiments, an anti-TIGIT antibody recognizes an epitope that comprises Phe at position 81 and Lys or Ser at position 82, and further comprises Thr at position 51, Ala at position 52, Glu or Gln at position 53, Val at position 54, and/or Thr at position 55. In some embodiments, an anti-TIGIT antibody recognizes an epitope that comprises Phe at position 81 and Lys or Ser at position 82, and further comprises Gly at position 74, Trp at position 75, His at position 76, and/or Val or Ile at position 77. In some embodiments, an anti-TIGIT antibody recognizes an epitope that comprises Phe at position 81 and Lys or Ser at position 82, and further comprises Pro at position 87, Gly at position 88, Pro at position 89, Ser or Gly at position 90, Leu at position 91, Gly at position 92, and/or Leu at position 93. In some embodiments, an anti-TIGIT antibody recognizes an epitope comprising the amino acid residues Thr51, Ala52, Gln53, Val54, Thr55, Gly74, Trp75, His76, Ile77, Phe81, Lys82, Pro87, Gly88, Pro89, Gly90, Leu91, Gly92, and Leu93.

In some embodiments, an anti-TIGIT antibody recognizes an epitope that comprises Phe at position 81 and Lys or Ser at position 82, and further comprises Ala at position 52 and/or Glu or Gln at position 53. In some embodiments, an anti-TIGIT antibody recognizes an epitope that comprises Phe at position 81 and Lys or Ser at position 82, and further comprises Leu at position 73, Gly at position 74, and/or Trp at position 75. In some embodiments, an anti-TIGIT antibody recognizes an epitope that comprises Phe at position 81 and Lys or Ser at position 82, and further comprises Asp at position 83, Arg at position 84, Val at position 85, and/or Val or Ala at position 86. In some embodiments, an anti-TIGIT antibody recognizes an epitope comprising the amino acid residues Ala52, Gln53, Leu73, Gly74, Trp75, Pro79, Phe81, Lys82, Asp83, Arg84, Val85, and Ala86.

In some embodiments, an anti-TIGIT antibody recognizes an epitope of human TIGIT comprising the sequence ICNADLGWHISPSFK (SEQ ID NO:258), which corresponds to residues 68-82 of human TIGIT (SEQ ID NO:218). In some embodiments, an anti-TIGIT antibody recognizes an epitope of human TIGIT consisting of the sequence

(SEQ ID NO: 258)
ICNADLGWHISPSFK.

### Anti-TIGIT Antibody Sequences

In some embodiments, an anti-TIGIT antibody that binds to human TIGIT and that optionally exhibits cross-reactivity with cynomolgus monkey TIGIT and/or mouse TIGIT comprises a light chain sequence, or a portion thereof, and/or a heavy chain sequence, or a portion thereof, derived from any of the following antibodies described herein: Clone 2, Clone 2C, Clone 3, Clone 5, Clone 13, Clone 13A, Clone 13B, Clone 13C, Clone 13D, Clone 14, Clone 16, Clone 16C, Clone 16D, Clone 16E, Clone 18, Clone 21, Clone 22, Clone 25, Clone 25A, Clone 25B, Clone 25C, Clone 25D, Clone 25E, Clone 27, or Clone 54. The amino acid sequences of the CDR, light chain variable domain (VL), and heavy chain variable domain (VH) of the anti-TIGIT antibodies Clone 2, Clone 2C, Clone 3, Clone 5, Clone 13, Clone 13A, Clone 13B, Clone 13C, Clone 13D, Clone 14, Clone 16, Clone 16C, Clone 16D, Clone 16E, Clone 18, Clone 21, Clone 22, Clone 25, Clone 25A, Clone 25B, Clone 25C, Clone 25D, Clone 25E, Clone 27, and Clone 54 are set forth in Table 3 below.

In some embodiments, an anti-TIGIT antibody comprises a heavy chain variable region (VH) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:1, SEQ ID NO:19, SEQ ID NO:37, SEQ ID NO:55, SEQ ID NO:73, SEQ ID NO:91, SEQ ID NO:109, SEQ ID NO:127, SEQ ID NO:145, SEQ ID NO:163, SEQ ID NO:181, SEQ ID NO:199, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, or SEQ ID NO:257. In some embodiments, an anti-TIGIT antibody comprises a VH comprising the amino acid sequence of SEQ ID NO:1, SEQ ID NO:19, SEQ ID NO:37, SEQ ID NO:55, SEQ ID NO:73, SEQ ID NO:91, SEQ ID NO:109, SEQ ID NO:127, SEQ ID NO:145, SEQ ID NO:163, SEQ ID NO:181, SEQ ID NO:199, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, or SEQ ID NO:257. In some embodiments, a VH sequence having at least 90% sequence identity to a reference sequence (e.g., SEQ ID NO:1, SEQ ID NO:19, SEQ ID NO:37, SEQ ID NO:55, SEQ ID NO:73, SEQ ID NO:91, SEQ ID NO:109, SEQ ID NO:127, SEQ ID NO:145, SEQ ID NO:163, SEQ ID NO:181, SEQ ID NO:199, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, or SEQ ID NO:257) contains one, two, three, four, five, six, seven, eight, nine, ten or more substitutions (e.g., conservative substitutions), insertions, or deletions relative to the reference sequence but retains the ability to bind to human TIGIT and optionally, retains the ability to block binding of CD155 and/or CD112 to TIGIT.

In some embodiments, an anti-TIGIT antibody comprises a light chain variable region (VL) comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:10, SEQ ID NO:28, SEQ ID NO:46, SEQ ID NO:64, SEQ ID NO:82, SEQ ID NO:100, SEQ ID NO:118, SEQ ID NO:136, SEQ ID NO:154, SEQ ID NO:172, SEQ ID NO:190, or SEQ ID NO:208. In some embodiments, an anti-TIGIT antibody comprises a VL comprising the amino acid sequence of SEQ ID NO:10, SEQ ID NO:28, SEQ ID NO:46, SEQ ID NO:64, SEQ ID NO:82, SEQ ID NO:100, SEQ ID NO:118, SEQ ID NO:136, SEQ ID NO:154, SEQ ID NO:172, SEQ ID NO:190, or SEQ ID NO:208. In some embodiments, a VL sequence having at least 90% sequence identity to a reference sequence (e.g., SEQ ID NO:10, SEQ ID NO:28, SEQ ID NO:46, SEQ ID NO:64, SEQ ID NO:82, SEQ ID NO:100, SEQ ID NO:118, SEQ ID NO:136, SEQ ID NO:154, SEQ ID NO:172, SEQ ID NO:190, or SEQ ID NO:208) contains one, two, three, four, five, six, seven, eight, nine, ten or more substitutions (e.g., conservative substitutions), insertions, or deletions relative to the reference sequence but retains the ability to bind to human TIGIT and optionally, retains the ability to block binding of CD155 and/or CD112 to TIGIT.

In some embodiments, an anti-TIGIT antibody comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:1, SEQ ID NO:19, SEQ ID NO:37, SEQ ID NO:55, SEQ ID NO:73, SEQ ID NO:91, SEQ ID NO:109, SEQ ID NO:127, SEQ ID NO:145, SEQ ID NO:163, SEQ ID NO:181, SEQ ID NO:199, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, or SEQ ID NO:257, and further comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:10, SEQ ID NO:28, SEQ ID NO:46, SEQ ID NO:64, SEQ ID NO:82, SEQ ID NO: 100, SEQ ID NO: 118, SEQ ID NO:136, SEQ ID NO:154, SEQ ID NO:172, SEQ ID NO:190, or SEQ ID NO:208. In some embodiments, an anti-TIGIT antibody comprises a heavy chain variable region comprising the amino acid sequence of SEQ ID NO:1, SEQ ID NO:19, SEQ ID NO:37, SEQ ID NO:55, SEQ ID NO:73, SEQ ID NO:91, SEQ ID NO:109, SEQ ID NO:127, SEQ ID NO:145, SEQ ID NO:163, SEQ ID NO:181, SEQ ID NO:199, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, or SEQ ID NO:257 and further comprises a light chain variable region comprising the amino acid sequence of SEQ ID NO:10, SEQ ID NO:28, SEQ ID NO:46, SEQ ID NO:64, SEQ ID NO:82, SEQ ID NO:100, SEQ ID NO:118, SEQ ID NO:136, SEQ ID NO:154, SEQ ID NO:172, SEQ ID NO:190, or SEQ ID NO:208.

In some embodiments, an anti-TIGIT antibody comprises:
(a) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:1 or SEQ ID NO:245 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:10;
(b) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:19 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:28;
(c) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:37 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:46;
(d) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NO:55, SEQ ID NO:246, SEQ ID NO:247, SEQ ID NO:248, or SEQ ID NO:249 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:64;
(e) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:73 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:82;
(f) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NO:91, SEQ ID NO:250, SEQ ID NO:251, or SEQ ID NO:252 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:100;
(g) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:109 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:118;
(h) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:127 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:136;
(i) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:145 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:154;
(j) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NO:163, SEQ ID NO:253, SEQ ID NO:254, SEQ ID NO:255, SEQ ID NO:256, or SEQ ID NO:257 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:172;
(k) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:181 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:190; or
(l) a VH comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:199 and a VL comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:208.

In some embodiments, an anti-TIGIT antibody comprises:
(a) a VH comprising the amino acid sequence of SEQ ID NO:1 and a VL comprising the amino acid sequence of SEQ ID NO:10;
(b) a VH comprising the amino acid sequence of SEQ ID NO:19 and a VL comprising the amino acid sequence of SEQ ID NO:28;
(c) a VH comprising the amino acid sequence of SEQ ID NO:37 and a VL comprising the amino acid sequence of SEQ ID NO:46;
(d) a VH comprising the amino acid sequence of SEQ ID NO:55 and a VL comprising the amino acid sequence of SEQ ID NO:64;
(e) a VH comprising the amino acid sequence of SEQ ID NO:73 and a VL comprising the amino acid sequence of SEQ ID NO:82;
(f) a VH comprising the amino acid sequence of SEQ ID NO:91 and a VL comprising the amino acid sequence of SEQ ID NO:100;
(g) a VH comprising the amino acid sequence of SEQ ID NO:109 and a VL comprising the amino acid sequence of SEQ ID NO:118;
(h) a VH comprising the amino acid sequence of SEQ ID NO:127 and a VL comprising the amino acid sequence of SEQ ID NO:136;
(i) a VH comprising the amino acid sequence of SEQ ID NO:145 and a VL comprising the amino acid sequence of SEQ ID NO:154;
(j) a VH comprising the amino acid sequence of SEQ ID NO:163 and a VL comprising the amino acid sequence of SEQ ID NO:172;
(k) a VH comprising the amino acid sequence of SEQ ID NO:181 and a VL comprising the amino acid sequence of SEQ ID NO:190;
(l) a VH comprising the amino acid sequence of SEQ ID NO:199 and a VL comprising the amino acid sequence of SEQ ID NO:208; or
(m) a VH comprising the amino acid sequence of SEQ ID NO:245 and a VL comprising the amino acid sequence of SEQ ID NO:10; or
(n) a VH comprising the amino acid sequence of SEQ ID NO:246 and a VL comprising the amino acid sequence of SEQ ID NO:64; or
(o) a VH comprising the amino acid sequence of SEQ ID NO:247 and a VL comprising the amino acid sequence of SEQ ID NO:64; or
(p) a VH comprising the amino acid sequence of SEQ ID NO:248 and a VL comprising the amino acid sequence of SEQ ID NO:64;
(q) a VH comprising the amino acid sequence of SEQ ID NO:249 and a VL comprising the amino acid sequence of SEQ ID NO:64; or
(r) a VH comprising the amino acid sequence of SEQ ID NO:250 and a VL comprising the amino acid sequence of SEQ ID NO:100; or
(s) a VH comprising the amino acid sequence of SEQ ID NO:251 and a VL comprising the amino acid sequence of SEQ ID NO:100; or
(t) a VH comprising the amino acid sequence of SEQ ID NO:252 and a VL comprising the amino acid sequence of SEQ ID NO:100; or
(u) a VH comprising the amino acid sequence of SEQ ID NO:253 and a VL comprising the amino acid sequence of SEQ ID NO:172; or
(v) a VH comprising the amino acid sequence of SEQ ID NO:254 and a VL comprising the amino acid sequence of SEQ ID NO:172; or
(w) a VH comprising the amino acid sequence of SEQ ID NO:255 and a VL comprising the amino acid sequence of SEQ ID NO:172; or
(x) a VH comprising the amino acid sequence of SEQ ID NO:256 and a VL comprising the amino acid sequence of SEQ ID NO:172; or
(y) a VH comprising the amino acid sequence of SEQ ID NO:257 and a VL comprising the amino acid sequence of SEQ ID NO:172.

In some embodiments, an anti-TIGIT antibody comprises one or more (e.g., one, two, three, four, five, or more) of:
a heavy chain CDR1 sequence comprising the amino acid sequence of any of SEQ ID NO:4, SEQ ID NO:22, SEQ ID NO:40, SEQ ID NO:58, SEQ ID NO:76, SEQ ID NO:94, SEQ ID NO: 112, SEQ ID NO:130, SEQ ID NO:148, SEQ ID NO:166, SEQ ID NO:184, SEQ ID NO:202, SEQ ID NO:221, SEQ ID NO:224, SEQ ID NO:226, SEQ ID NO:231, SEQ ID NO:233, SEQ ID NO:239, or SEQ ID NO:243;
a heavy chain CDR2 sequence comprising the amino acid sequence of any of SEQ ID NO:6, SEQ ID NO:24, SEQ ID NO:42, SEQ ID NO:60, SEQ ID NO:78, SEQ ID NO:96, SEQ ID NO: 114, SEQ ID NO:132, SEQ ID NO:150, SEQ ID NO:168, SEQ ID NO:186, SEQ ID NO:204, SEQ ID NO:222, SEQ ID NO:225, SEQ ID NO:227, SEQ ID NO:229, SEQ ID NO:232, SEQ ID NO:234, SEQ ID NO:238, or SEQ ID NO:240;
a heavy chain CDR3 sequence comprising the amino acid sequence of any of SEQ ID NO:8, SEQ ID NO:26, SEQ ID NO:44, SEQ ID NO:62, SEQ ID NO:80, SEQ ID NO:98, SEQ ID NO: 116, SEQ ID NO:134, SEQ ID NO:152, SEQ ID NO:170, SEQ ID NO:188, SEQ ID NO:206, SEQ ID NO:223, SEQ ID NO:228, SEQ ID NO:230, SEQ ID NO:235, SEQ ID NO:236, SEQ ID NO:237, SEQ ID NO:241, SEQ ID NO:242, or SEQ ID NO:244;
a light chain CDR1 sequence comprising the amino acid sequence of any of SEQ ID NO:13, SEQ ID NO:31, SEQ ID NO:49, SEQ ID NO:67, SEQ ID NO:85, SEQ ID NO:103, SEQ ID NO:121, SEQ ID NO:139, SEQ ID NO:157, SEQ ID NO:175, SEQ ID NO:193, or SEQ ID NO:211;
a light chain CDR2 sequence comprising the amino acid sequence of any of SEQ ID NO:15, SEQ ID NO:33, SEQ ID NO:51, SEQ ID NO:69, SEQ ID NO:87, SEQ ID NO:105, SEQ ID NO:123, SEQ ID NO:141, SEQ ID NO:159, SEQ ID NO:177, SEQ ID NO:195, or SEQ ID NO:213; and/or
a light chain CDR3 sequence comprising the amino acid sequence of any of SEQ ID NO:17, SEQ ID NO:35, SEQ ID NO:53, SEQ ID NO:71, SEQ ID NO:89, SEQ ID NO:107, SEQ ID NO:125, SEQ ID NO:143, SEQ ID NO:161, SEQ ID NO:179, SEQ ID NO:197, or SEQ ID NO:215.

In some embodiments, an anti-TIGIT antibody comprises a heavy chain CDR1 sequence comprising the amino acid sequence of any of SEQ ID NO:4, SEQ ID NO:22, SEQ ID NO:40, SEQ ID NO:58, SEQ ID NO:76, SEQ ID NO:94, SEQ ID NO:112, SEQ ID NO:130, SEQ ID NO:148, SEQ ID NO:166, SEQ ID NO:184, SEQ ID NO:202, SEQ ID NO:221, SEQ ID NO:224, SEQ ID NO:226, SEQ ID NO:231, SEQ ID NO:233, SEQ ID NO:239, or SEQ ID NO:243; a heavy chain CDR2 sequence comprising the amino acid sequence of any of SEQ ID NO:6, SEQ ID NO:24, SEQ ID NO:42, SEQ ID NO:60, SEQ ID NO:78, SEQ ID NO:96, SEQ ID NO:114, SEQ ID NO:132, SEQ ID NO:150, SEQ ID NO:168, SEQ ID NO:186, SEQ ID NO:204, SEQ ID NO:222, SEQ ID NO:225, SEQ ID NO:227, SEQ ID NO:229, SEQ ID NO:232, SEQ ID NO:234, SEQ ID NO:238, or SEQ ID NO:240; and a heavy chain CDR3 sequence comprising the amino acid sequence of any of SEQ ID NO:8, SEQ ID NO:26, SEQ ID NO:44, SEQ ID NO:62, SEQ ID NO:80, SEQ ID NO:98, SEQ ID NO: 116, SEQ ID NO:134, SEQ ID NO:152, SEQ ID NO:170, SEQ ID NO:188, SEQ ID NO:206, SEQ ID NO:223, SEQ ID NO:228, SEQ ID NO:230, SEQ ID NO:235, SEQ ID NO:236, SEQ ID NO:237, SEQ ID NO:241, SEQ ID NO:242, or SEQ ID NO:244.

In some embodiments, an anti-TIGIT antibody comprises a light chain CDR1 sequence comprising the amino acid sequence of any of SEQ ID NO:13, SEQ ID NO:31, SEQ ID NO:49, SEQ ID NO:67, SEQ ID NO:85, SEQ ID NO:103, SEQ ID NO:121, SEQ ID NO:139, SEQ ID NO:157, SEQ ID NO:175, SEQ ID NO:193, or SEQ ID NO:211; a light chain CDR2 sequence comprising the amino acid sequence of any of SEQ ID NO:15, SEQ ID NO:33, SEQ ID NO:51, SEQ ID NO:69, SEQ ID NO:87, SEQ ID NO:105, SEQ ID NO:123, SEQ ID NO:141, SEQ ID NO:159, SEQ ID NO:177, SEQ ID NO:195, or SEQ ID NO:213; and a light chain CDR3 sequence comprising the amino acid sequence of any of SEQ ID NO:17, SEQ ID NO:35, SEQ ID NO:53, SEQ ID NO:71, SEQ ID NO:89, SEQ ID NO:107, SEQ ID NO:125, SEQ ID NO:143, SEQ ID NO:161, SEQ ID NO:179, SEQ ID NO:197, or SEQ ID NO: 215.

In some embodiments, an anti-TIGIT antibody comprises:
(i) a heavy chain CDR1 sequence comprising the amino acid sequence of any of SEQ ID NO:4, SEQ ID NO:22, SEQ ID NO:40, SEQ ID NO:58, SEQ ID NO:76, SEQ ID NO:94, SEQ ID NO: 112, SEQ ID NO:130, SEQ ID NO:148, SEQ ID NO:166, SEQ ID NO:184, SEQ ID NO:202, SEQ ID NO:221, SEQ ID NO:224, SEQ ID NO:226, SEQ ID NO:231, SEQ ID NO:233, SEQ ID NO:239, or SEQ ID NO:243; and
(ii) a heavy chain CDR2 sequence comprising the amino acid sequence of any of SEQ ID NO:6, SEQ ID NO:24, SEQ ID NO:42, SEQ ID NO:60, SEQ ID NO:78, SEQ ID NO:96, SEQ ID NO: 114, SEQ ID NO: 132, SEQ ID NO:150, SEQ ID NO:168, SEQ ID NO:186, SEQ ID NO:204, SEQ ID NO:222, SEQ ID NO:225, SEQ ID NO:227, SEQ ID NO:229, SEQ ID NO:232, SEQ ID NO:234, SEQ ID NO:238, or SEQ ID NO:240; and
(iii) a heavy chain CDR3 sequence comprising the amino acid sequence of any of SEQ ID NO:8, SEQ ID NO:26, SEQ ID NO:44, SEQ ID NO:62, SEQ ID NO:80, SEQ ID NO:98, SEQ ID NO: 116, SEQ ID NO:134, SEQ ID NO:152, SEQ ID NO:170, SEQ ID NO:188, SEQ ID NO:206, SEQ ID NO:223, SEQ ID NO:228, SEQ ID NO:230, SEQ ID NO:235, SEQ ID NO:236, SEQ ID NO:237, SEQ ID NO:241, SEQ ID NO:242, or SEQ ID NO:244; and
(iv) a light chain CDR1 sequence comprising the amino acid sequence of any of SEQ ID NO:13, SEQ ID NO:31, SEQ ID NO:49, SEQ ID NO:67, SEQ ID NO:85, SEQ ID NO:103, SEQ ID NO:121, SEQ ID NO:139, SEQ ID NO:157, SEQ ID NO:175, SEQ ID NO:193, or SEQ ID NO: 211; and
(v) a light chain CDR2 sequence comprising the amino acid sequence of any of SEQ ID NO:15, SEQ ID NO:33, SEQ ID NO:51, SEQ ID NO:69, SEQ ID NO:87, SEQ ID NO:105, SEQ ID NO:123, SEQ ID NO:141, SEQ ID NO:159, SEQ ID NO:177, SEQ ID NO:195, or SEQ ID NO:213; and
(vi) a light chain CDR3 sequence comprising the amino acid sequence of any of SEQ ID NO:17, SEQ ID NO:35, SEQ ID NO:53, SEQ ID NO:71, SEQ ID NO:89, SEQ ID NO:107, SEQ ID NO: 125, SEQ ID NO:143, SEQ ID NO:161, SEQ ID NO:179, SEQ ID NO:197, or SEQ ID NO: 215.

In some embodiments, an anti-TIGIT antibody comprises: (i) a heavy chain CDR1 sequence comprising the amino acid sequence of SEQ ID NO:4 or SEQ ID NO:221; (ii) a heavy chain CDR2 sequence comprising the amino acid sequence of SEQ ID NO:6 or SEQ ID NO:222; (iii) a heavy chain CDR3 sequence comprising the amino acid sequence of SEQ ID NO:8 or SEQ ID NO:223; (iv) a light chain CDR1 sequence comprising the amino acid sequence of SEQ ID NO: 13; (v) a light chain CDR2 sequence comprising the amino acid sequence of SEQ ID NO:15; and (vi) a light chain CDR3 sequence comprising the amino acid sequence of SEQ ID NO:17.

In some embodiments, an anti-TIGIT antibody comprises: (i) a heavy chain CDR1 sequence comprising the amino acid sequence of any of SEQ ID NO:58, SEQ ID NO:224, or SEQ ID NO:226; (ii) a heavy chain CDR2 sequence comprising the amino acid sequence of any of SEQ ID NO:60, SEQ ID NO:225, SEQ ID NO:227, or SEQ ID NO:229; (iii) a heavy chain CDR3 sequence comprising the amino acid sequence of any of SEQ ID NO:62, SEQ ID NO:228, or SEQ ID NO:230; (iv) a light chain CDR1 sequence comprising the amino acid sequence of SEQ ID NO:67; (v) a light chain CDR2 sequence comprising the amino acid sequence of SEQ ID NO:69; and (vi) a light chain CDR3 sequence comprising the amino acid sequence of SEQ ID NO:71.

In some embodiments, an anti-TIGIT antibody comprises: (i) a heavy chain CDR1 sequence comprising the amino acid sequence of any of SEQ ID NO:94, SEQ ID NO:231, or SEQ ID NO:233; (ii) a heavy chain CDR2 sequence comprising the amino acid sequence of any of SEQ ID NO:96, SEQ ID NO:232, or SEQ ID NO:234; (iii) a heavy chain CDR3 sequence comprising the amino acid sequence of any of SEQ ID NO:98, SEQ ID NO:235, SEQ ID NO:236, or SEQ ID NO:237; (iv) a light chain CDR1 sequence comprising the amino acid sequence of SEQ ID NO:103; (v) a light chain CDR2 sequence comprising the amino acid sequence of SEQ ID NO:105; and (vi) a light chain CDR3 sequence comprising the amino acid sequence of SEQ ID NO: 107.

In some embodiments, an anti-TIGIT antibody comprises: (i) a heavy chain CDR1 sequence comprising the amino acid sequence of any of SEQ ID NO:166, SEQ ID NO:239, or SEQ ID NO:243; (ii) a heavy chain CDR2 sequence comprising the amino acid sequence of any of SEQ ID NO:168, SEQ ID NO:238, or SEQ ID NO:240; (iii) a heavy chain CDR3 sequence comprising the amino acid sequence of any of SEQ ID NO:170, SEQ ID NO:241, SEQ ID NO:242, or SEQ ID NO:244; (iv) a light chain CDR1 sequence comprising the amino acid sequence of SEQ ID NO:175; (v) a light chain CDR2 sequence comprising the amino acid sequence of SEQ ID NO: 177; and (vi) a light chain CDR3 sequence comprising the amino acid sequence of SEQ ID NO: 179.

In some embodiments, an anti-TIGIT antibody comprises a heavy chain CDR1-3 and a light chain CDR1-3 comprising the amino acid sequences of:
(a) SEQ ID NOs: 4, 6, 8, 13, 15, and 17, respectively;
(b) SEQ ID NOs: 22, 24, 26, 31, 33, and 35, respectively;
(c) SEQ ID NOs: 40, 42, 44, 49, 51, and 53, respectively;
(d) SEQ ID NOs: 58, 60, 62, 67, 69, and 71, respectively;
(e) SEQ ID NOs: 76, 78, 80, 85, 87, and 89, respectively;
(f) SEQ ID NOs: 94, 96, 98, 103, 105, and 107, respectively;
(g) SEQ ID NOs: 112, 114, 116, 121, 123, and 125, respectively;
(h) SEQ ID NOs: 130, 132, 134, 139, 141, and 143, respectively;
(i) SEQ ID NOs: 148, 150, 152, 157, 159, and 161, respectively;
(j) SEQ ID NOs: 166, 168, 170, 175, 177, and 179, respectively;
(k) SEQ ID NOs: 184, 186, 188, 193, 195, and 197, respectively;
(l) SEQ ID NOs: 202, 204, 206, 211, 213, and 215, respectively; or
(m) SEQ ID NOs: 221, 222, 223, 13, 15, and 17, respectively; or
(n) SEQ ID NOs: 224, 225, 62, 67, 69, and 71, respectively; or
(o) SEQ ID NOs: 226, 227, 228, 67, 69, and 71, respectively; or
(p) SEQ ID NOs: 224, 229, 230, 67, 69, and 71, respectively; or
(q) SEQ ID NOs: 224, 227, 230, 67, 69, and 71, respectively; or
(r) SEQ ID NOs: 231, 232, 235, 103, 105, and 107, respectively; or
(s) SEQ ID NOs: 233, 234, 236, 103, 105, and 107, respectively; or
(t) SEQ ID NOs: 233, 234, 237, 103, 105, and 107, respectively; or
(u) SEQ ID NOs: 166, 238, 170, 175, 177, and 179, respectively; or
(v) SEQ ID NOs: 239, 240, 170, 175, 177, and 179, respectively; or
(w) SEQ ID NOs: 239, 240, 241, 175, 177, and 179, respectively; or
(x) SEQ ID NOs: 239, 240, 242, 175, 177, and 179, respectively; or
(y) SEQ ID NOs: 243, 168, 244, 175, 177, and 179, respectively

In some embodiments, the antibody further includes a framework, such as a human immunoglobulin framework. For example, in some embodiments, an antibody comprises a CDR as described herein and further comprises an acceptor human framework, e.g., a human immunoglobulin framework or a human consensus framework. Human immunoglobulin frameworks may be part of the human antibody, or a non-human antibody may be humanized by replacing one or more endogenous frameworks with human framework region(s). Human framework regions that may be used for humanization include but are not limited to: framework regions selected using the “best-fit” method (see, e.g., Sims et al., J. Immunol. 151:2296 (1993)); framework regions derived from the consensus sequence of human antibodies of a particular subgroup of light or heavy chain variable regions (see, e.g., Carter et al., Proc. Natl. Acad. Sci. USA, 89:4285 (1992); and Presta et al., J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J Biol. Chem. 272:10678-10684 (1997) and Rosok et al., J Biol. Chem. 271:22611-22618 (1996)). Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

In some embodiments, an anti-TIGIT antibody comprises one or more heavy chain framework regions (FR1, FR2, FR3, and/or FR4) comprising an amino acid sequence of SEQ ID NO:3, SEQ ID NO:5, SEQ ID NO:7, SEQ ID NO:9, SEQ ID NO:21, SEQ ID NO:23, SEQ ID NO:25, SEQ ID NO:27, SEQ ID NO:39, SEQ ID NO:41, SEQ ID NO:43, SEQ ID NO:45, SEQ ID NO:57, SEQ ID NO:59, SEQ ID NO:61, SEQ ID NO:63, SEQ ID NO:75, SEQ ID NO:77, SEQ ID NO:79, SEQ ID NO:81, SEQ ID NO:93, SEQ ID NO:95, SEQ ID NO:97, SEQ ID NO:99, SEQ ID NO:111, SEQ ID NO:113, SEQ ID NO:115, SEQ ID NO:117, SEQ ID NO:129, SEQ ID NO:131, SEQ ID NO:133, SEQ ID NO:135, SEQ ID NO:147, SEQ ID NO:149, SEQ ID NO:151, SEQ ID NO:153, SEQ ID NO:165, SEQ ID NO:167, SEQ ID NO:169, SEQ ID NO:171, SEQ ID NO:183, SEQ ID NO:185, SEQ ID NO:187, SEQ ID NO:189, SEQ ID NO:201, SEQ ID NO:203, SEQ ID NO:205, or SEQ ID NO:207.

In some embodiments, an anti-TIGIT antibody comprises one or more light chain framework regions (FR1, FR2, FR3, and/or FR4) comprising an amino acid sequence of SEQ ID NO:12, SEQ ID NO:14, SEQ ID NO:16, SEQ ID NO:18, SEQ ID NO:30, SEQ ID NO:32, SEQ ID NO:34, SEQ ID NO:36, SEQ ID NO:48, SEQ ID NO:50, SEQ ID NO:52, SEQ ID NO:54, SEQ ID NO:66, SEQ ID NO:68, SEQ ID NO:70, SEQ ID NO:72, SEQ ID NO:84, SEQ ID NO:86, SEQ ID NO:88, SEQ ID NO:90, SEQ ID NO:102, SEQ ID NO:104, SEQ ID NO:106, SEQ ID NO:108, SEQ ID NO:120, SEQ ID NO:122, SEQ ID NO:124, SEQ ID NO:126, SEQ ID NO:138, SEQ ID NO:140, SEQ ID NO:142, SEQ ID NO:144, SEQ ID NO:156, SEQ ID NO:158, SEQ ID NO:160, SEQ ID NO:162, SEQ ID NO:174, SEQ ID NO:176, SEQ ID NO:178, SEQ ID NO:180, SEQ ID NO:192, SEQ ID NO:194, SEQ ID NO:196, SEQ ID NO:198, SEQ ID NO:210, SEQ ID NO:212, SEQ ID NO:214, or SEQ ID NO:216.

In some embodiments, the anti-TIGIT antibodies of the instant disclosure do not compete for binding with the antibodies described in US 2009/0258013, US 2016/0176963, US 2016/0376365, or WO 2016/028656. In some embodiments, the anti-TIGIT antibodies of the instant disclosure do not bind to the same epitope as antibodies described in US 2009/0258013, US 2016/0176963, US 2016/0376365, or WO 2016/028656.

### Preparation of Antibodies

For preparing an antibody that binds to TIGIT, many techniques known in the art can be used. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)).

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al. (2010) PEDS 23:311; and Chao et al., Nature Protocols, 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379; WO 2010/105256; and WO 2012/009568. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,946,778, 4,816,567) can also be adapted to produce antibodies. Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or antibodies covalently bound to immunotoxins (see, e.g., U.S. Pat. No. 4,676,980, WO 91/00360; and WO 92/200373).

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or be under the control of different promoters. In other embodiments, the V_{H} and V_{L} region may be expressed using separate vectors. A VH or VL region as described herein may optionally comprise a methionine at the N-terminus.

Methods for humanizing or primatizing non-human antibodies are also known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. Transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)).

As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)2, a scFv, or a diabody) are generated. Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, antibody fragments can be isolated from antibody phage libraries. Alternatively, Fab′-SH fragments can be directly recovered from E. coli cells and chemically coupled to form F(ab′)₂ fragments (see, e.g., Carter et al., BioTechnology, 10:163-167 (1992)). According to another approach, F(ab′)₂ fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870.

In some embodiments, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007).

In some embodiments, multispecific antibodies comprising an anti-TIGIT antibody or antigen-binding fragment as described herein are provided, e.g., a bispecific antibody. Multispecific antibodies are antibodies that have binding specificities for at least two different sites. In some embodiments, the multispecific antibody has a binding specificity for TIGIT (e.g., human TIGIT) and has a binding specificity for at least one other antigen. Methods for making multispecific antibodies include, but are not limited to, recombinant co-expression of two pairs of heavy chain and light chain in a host cell (see, e.g., Zuo et al., Protein Eng Des Sel, 2000, 13:361-367); “knobs-into-holes” engineering (see, e.g., Ridgway et al., Protein Eng Des Sel, 1996, 9:617-721); “diabody” technology (see, e.g., Hollinger et al., PNAS (USA), 1993, 90:6444-6448); and intramolecular trimerization (see, e.g., Alvarez-Cienfuegos et al., Scientific Reports, 2016, doi:/10.1038/srep28643); See also, Spiess et al., Molecular Immunology, 2015, 67(2), Part A:95-106.

In some embodiments, antibody-drug conjugates comprising an anti-TIGIT antibody or antigen-binding fragment as described herein are provided. In antibody-drug conjugates, a monoclonal antibody having a binding specificity for an antigen (e.g., TIGIT) is covalently linked to a cytotoxic drug. Methods for making antibody-drug conjugates are described, e.g., in Chudasama et al., Nature Chemistry, 2016, 8:114-119; WO 2013/068874; and U.S. Pat. No. 8,535,678.

### Nucleic Acids, Vectors, and Host Cells

In some embodiments, the anti-TIGIT antibodies as described herein are prepared using recombinant methods. Accordingly, in some aspects, the invention provides isolated nucleic acids comprising a nucleic acid sequence encoding any of the anti-TIGIT antibodies as described herein (e.g., any one or more of the CDRs described herein); vectors comprising such nucleic acids; and host cells into which the nucleic acids are introduced that are used to replicate the antibody-encoding nucleic acids and/or to express the antibodies. In some embodiments, the host cell is eukaryotic, e.g., a Chinese Hamster Ovary (CHO) cell; or a human cell.

In some embodiments, a polynucleotide (e.g., an isolated polynucleotide) comprises a nucleotide sequence encoding an antibody or antigen-binding portion thereof as described herein (e.g., as described in the Section above entitled “Anti-TIGIT Antibody Sequences”). In some embodiments, the polynucleotide comprises a nucleotide sequence encoding one or more amino acid sequences (e.g., CDR, heavy chain, light chain, and/or framework regions) disclosed in Table 3 below. In some embodiments, the polynucleotide comprises a nucleotide sequence encoding an amino acid sequence having at least 85% sequence identity (e.g., at least 85%, at least 90%, at least 91%, at least 92%, at least 93%, at least 94%, at least 95%, at least 96%, at least 97%, at least 98%, or at least 99% sequence identity) to a sequence (e.g., a CDR, heavy chain, light chain, or framework region sequence) disclosed in Table 3 below.

In some embodiments, a polynucleotide (e.g., an isolated polynucleotide) comprises a nucleotide sequence encoding a heavy chain variable region as described herein. In some embodiments, a polynucleotide comprises a nucleotide sequence encoding a heavy chain variable region comprising an amino acid sequence that has at least 90% sequence identity to SEQ ID NO:1, SEQ ID NO:19, SEQ ID NO:37, SEQ ID NO:55, SEQ ID NO:73, SEQ ID NO:91, SEQ ID NO:109, SEQ ID NO:127, SEQ ID NO:145, SEQ ID NO:163, SEQ ID NO:181, SEQ ID NO:199, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, or SEQ ID NO:257. In some embodiments, the polynucleotide comprises the nucleotide sequence of SEQ ID NO:2, SEQ ID NO:20, SEQ ID NO:38, SEQ ID NO:56, SEQ ID NO:74, SEQ ID NO:92, SEQ ID NO:110, SEQ ID NO:128, SEQ ID NO:146, SEQ ID NO:164, SEQ ID NO:182, or SEQ ID NO:200.

In some embodiments, a polynucleotide (e.g., an isolated polynucleotide) comprises a nucleotide sequence encoding a light chain variable region as described herein. In some embodiments, a polynucleotide comprises a nucleotide sequence encoding a light chain variable region comprising an amino acid sequence that has at least 90% sequence identity to SEQ ID NO:10, SEQ ID NO:28, SEQ ID NO:46, SEQ ID NO:64, SEQ ID NO:82, SEQ ID NO:100, SEQ ID NO:118, SEQ ID NO:136, SEQ ID NO:154, SEQ ID NO:172, SEQ ID NO:190, or SEQ ID NO:208. In some embodiments, a polynucleotide comprises the nucleotide sequence of SEQ ID NO:11, SEQ ID NO:29, SEQ ID NO:47, SEQ ID NO:65, SEQ ID NO:83, SEQ ID NO:101, SEQ ID NO: 119, SEQ ID NO:137, SEQ ID NO:155, SEQ ID NO:173, SEQ ID NO:191, or SEQ ID NO: 209.

In some embodiments, the polynucleotide comprises a nucleotide sequence encoding comprises a nucleotide sequence encoding a heavy chain variable region and a light chain variable region as described herein. In some embodiments, a polynucleotide comprises a nucleotide sequence encoding a heavy chain variable region comprising an amino acid sequence that has at least 90% sequence identity to SEQ ID NO:1, SEQ ID NO:19, SEQ ID NO:37, SEQ ID NO:55, SEQ ID NO:73, SEQ ID NO:91, SEQ ID NO:109, SEQ ID NO:127, SEQ ID NO:145, SEQ ID NO:163, SEQ ID NO:181, SEQ ID NO:199, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, or SEQ ID NO:257 and encoding a light chain variable region comprising an amino acid sequence that has at least 90% sequence identity to SEQ ID NO:10, SEQ ID NO:28, SEQ ID NO:46, SEQ ID NO:64, SEQ ID NO:82, SEQ ID NO:100, SEQ ID NO:118, SEQ ID NO:136, SEQ ID NO:154, SEQ ID NO:172, SEQ ID NO:190, or SEQ ID NO:208. In some embodiments, the polynucleotide comprises the nucleotide sequence of SEQ ID NO:2, SEQ ID NO:20, SEQ ID NO:38, SEQ ID NO:56, SEQ ID NO:74, SEQ ID NO:92, SEQ ID NO:110, SEQ ID NO:128, SEQ ID NO:146, SEQ ID NO:164, SEQ ID NO:182, or SEQ ID NO:200, and further comprises the nucleotide sequence of SEQ ID NO:11, SEQ ID NO:29, SEQ ID NO:47, SEQ ID NO:65, SEQ ID NO:83, SEQ ID NO:101, SEQ ID NO: 119, SEQ ID NO:137, SEQ ID NO:155, SEQ ID NO:173, SEQ ID NO:191, or SEQ ID NO:209.

In a further aspect, methods of making an anti-TIGIT antibody as described herein are provided. In some embodiments, the method includes culturing a host cell as described herein (e.g., a host cell expressing a polynucleotide or vector as described herein) under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColEl, pCR1, RP4, phage DNAs, and shuttle vectors such as pSA3 and pAT28. Cloning vectors are available from commercial vendors such as BioRad, Stratagene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

### IV. Therapeutic Methods Using Anti-TIGIT Antibodies

In another aspect, methods for treating or preventing a cancer in a subject are provided. In some embodiments, the method comprises administering to the subject a therapeutic amount of an anti-TIGIT antibody or antigen binding fragment as described herein or a pharmaceutical composition comprising an anti-TIGIT antibody or antigen binding fragment as described herein. In some embodiments, the subject is a human, e.g., a human adult or a human child.

In some embodiments, the cancer is a cancer or cancer cell that is enriched for expression of CD112 and/or CD155. In some embodiments, CD112- and/or CD155-enriched cancers are identified by immunohistochemistry assessment of tumor samples using antibodies specific for CD112 or CD155. In some embodiments, CD112 or CD155 expression is enriched or increased in tumor cells or in tumor infiltrating leukocytes. In some embodiments, the cancer is identified based on the assessment of CD112 and/or CD155 mRNA levels in tumor samples (e.g., by methods known in the art such as quantitative RT-PCR). In some embodiments, measurements of soluble CD112 or CD155 in blood samples obtained from cancer patients may be used to identify a cancer that is enriched for expression of CD112 and/or CD155. In some embodiments, the method comprises obtaining a sample from a subject (e.g., a tumor sample or a blood sample), measuring the level of CD112 and/or CD155 in the sample from the subject, and comparing the level of CD112 and/or CD155 in the sample from the subject to a control value (e.g., a sample from a healthy control subject or a level of CD112 and/or CD155 expression determined for a population of healthy controls). In some embodiments, the method comprises determining that the level of CD112 and/or CD155 in the sample from the subject is higher than a control value, and subsequently administering to the subject an anti-TIGIT antibody as described herein.

In some embodiments, the cancer is a cancer or cancer cell that is enriched for T cells or natural killer (NK) cells that express TIGIT. In some embodiments, TIGIT-enriched cancers are identified by immunohistochemistry assessment of tumor samples using antibodies specific for TIGIT. In some embodiments, an antibody that is specific for T cells or NK cells (e.g., anti-CD3, anti-CD4, anti-CD8, anti-CD25, or anti-CD56) is used to determine a subset or subsets of tumor infiltrating cells that express TIGIT. In some embodiments, the cancer is identified based on the assessment of TIGIT mRNA levels in tumor samples. In some embodiments, measurements of soluble TIGIT in blood samples obtained from cancer patients may be used (optionally in combination with an antibody that is specific for T cells or NK cells) to identify a cancer that is enriched for T cells or NK cells that express TIGIT. In some embodiments, the method comprises obtaining a sample from a subject (e.g., a tumor sample or a blood sample), measuring the level of TIGIT in the sample from the subject, optionally detecting the presence of T cells or NK cells (e.g., using an antibody that is specific for T cells or NK cells such as anti-CD3, anti-CD4, anti-CD8, anti-CD25, or anti-CD56) and comparing the level of TIGIT in the sample from the subject to a control value (e.g., a sample from a healthy control subject or a level of TIGIT expression determined for a population of healthy controls). In some embodiments, the method comprises determining that the level of TIGIT in the sample from the subject is higher than a control value, and subsequently administering to the subject an anti-TIGIT antibody as described herein.

In some embodiments, the cancer is bladder cancer, breast cancer, uterine cancer, cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer (e.g., melanoma), neoplasm of the central nervous system, lymphoma, leukemia, myeloma, or sarcoma. In some embodiments, the cancer is stomach cancer. In some embodiments, the cancer is lung cancer. In some embodiments, the cancer is skin cancer (e.g., melanoma). In some embodiments, the cancer is a metastatic cancer. In some embodiments, the cancer is a lymphoma or a leukemia, including but not limited to acute myeloid, chronic myeloid, acute lymphocytic or chronic lymphocytic leukemia, diffuse large B-cell lymphoma, follicular lymphoma, mantle cell lymphoma, small lymphocytic lymphoma, primary mediastinal large B-cell lymphoma, splenic marginal zone B-cell lymphoma, or extranodal marginal zone B-cell lymphoma.

In some embodiments, the method further comprises administering to the subject a therapeutic amount of an immuno-oncology agent. In some embodiments, the immuno-oncology agent is an agent (e.g., an antibody, small molecule, or peptide) that antagonizes or inhibits a component of an immune checkpoint pathway, such as the PD-1 pathway, the CTLA-4 pathway, the Lag3 pathway, or the TIM-3 pathway. In some embodiments, the immuno-oncology agent is an agonist of a T cell coactivator (i.e., an agonist of a protein that stimulates T cell activation) by targeting the OX-40 pathway, the 4-1BB (CD137) pathway, the CD27 pathway, the ICOS pathway, or the GITR pathway.

In some embodiments, the immuno-oncology agent is a PD-1 pathway inhibitor. In some embodiments, the PD-1 pathway inhibitor is an anti-PD-1 antibody or anti-PD-L1 antibody, such as but not limited to pembrolizumab, nivolumab, durvalumab, pidilizumab, or atezolizumab. PD-1 pathway inhibitors are described in the art. See, e.g., Dolan et al., Cancer Control, 2014, 21:231-237; Luke et al., Oncotarget, 2014, 6:3479-3492; US 2016/0222113; US 2016/0272708; US 2016/0272712; and US 2016/0319019.

In some embodiments, the immuno-oncology agent is an agonist of a T cell coactivator. In some embodiments, the immuno-oncology agent is an agonist of CD28, CD28H, CD3, 4-1BB (CD137), ICOS, OX40, GITR, CD27, or CD40. In some embodiments, the immuno-oncology agent is an immune stimulatory cytokine. In some embodiments, the immune stimulatory cytokine is granulocyte-macrophage colony stimulating factor (GM-CSF), macrophage colony stimulating factor (M-CSF), granulocyte colony stimulating factor (G-CSF), interleukin 1 (IL-1), interleukin 2 (IL-2), interleukin 3 (IL-3), interleukin 12 (IL-12), interleukin 15 (IL-15), or interferon gamma (IFN-7).

In some embodiments, treatment with an anti-TIGIT antibody as described herein is combined with one or more other cancer treatments, such as surgery, radiation, or chemotherapy. In some embodiments, the chemotherapeutic agent is an alkylating agent (e.g., cyclophosphamide, ifosfamide, chlorambucil, busulfan, melphalan, mechlorethamine, uramustine, thiotepa, nitrosoureas, or temozolomide), an anthracycline (e.g., doxorubicin, adriamycin, daunorubicin, epirubicin, or mitoxantrone), a cytoskeletal disruptor (e.g., paclitaxel or docetaxel), a histone deacetylase inhibitor (e.g., vorinostat or romidepsin), an inhibitor of topoisomerase (e.g., irinotecan, topotecan, amsacrine, etoposide, or teniposide), a kinase inhibitor (e.g., bortezomib, erlotinib, gefitinib, imatinib, vemurafenib, or vismodegib), a nucleoside analog or precursor analog (e.g., azacitidine, azathioprine, capecitabine, cytarabine, fluorouracil, gemcitabine, hydroxyurea, mercaptopurine, methotrexate, or thioguanine), a peptide antibiotic (e.g., actinomycin or bleomycin), a platinum-based agent (e.g., cisplatin, oxaloplatin, or carboplatin), or a plant alkaloid (e.g., vincristine, vinblastine, vinorelbine, vindesine, podophyllotoxin, paclitaxel, or docetaxel).

In some embodiments, the anti-TIGIT antibody (and optionally an immuno-oncology agent or other therapeutic treatment) is administered at a therapeutically effective amount or dose. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of an anti-TIGIT antibody or pharmaceutical composition comprising an anti-TIGIT antibody (and optionally an immuno-oncology agent or other therapeutic treatment) can be oral, intraperitoneal, transdermal, subcutaneous, intravenous, intramuscular, inhalational, topical, intralesional, rectal, intrabronchial, nasal, transmucosal, intestinal, ocular or otic delivery, or any other methods known in the art. In some embodiments, the anti-TIGIT antibody (and optionally an immuno-oncology agent) is administered orally, intravenously, or intraperitoneally.

Co-administered therapeutic agents (e.g., the anti-TIGIT antibody and an immuno-oncology agent or other therapeutic treatment) can be administered together or separately, simultaneously or at different times. When administered, the therapeutic agents independently can be administered once, twice, three, four times daily or more or less often, as needed. In some embodiments, the administered therapeutic agents are administered once daily. In some embodiments, the administered therapeutic agents are administered at the same time or times, for instance as an admixture. In some embodiments, one or more of the therapeutic agents is administered in a sustained-release formulation.

In some embodiments, the anti-TIGIT antibody and another therapeutic treatment (e.g., an immuno-oncology agent) are administered concurrently. In some embodiments, the anti-TIGIT antibody and another therapeutic treatment (e.g., an immuno-oncology agent) are administered sequentially. For example, in some embodiments, an anti-TIGIT antibody is administered first, for example for about 1, 2, 3, 4, 5, 6, 7, 8, 9, 10, 15, 20, 25, 30, 40, 50, 60, 70, 80, 90, 100 days or more prior to administering an immuno-oncology agent. In some embodiments, an immuno-oncology agent is administered first, for example for about 1, 2, 3, 4, 5, 6, 7, 8, 9, 10, 15, 20, 25, 30, 40, 50, 60, 70, 80, 90, 100 days or more prior to administering an anti-TIGIT antibody.

In some embodiments, the anti-TIGIT antibody (and optionally the immuno-oncology agent) is administered to the subject over an extended period of time, e.g., for at least 30, 40, 50, 60, 70, 80, 90, 100, 150, 200, 250, 300, 350 days or longer.

### V. Compositions and Kits

In another aspect, compositions and kits comprising an anti-TIGIT antibody for use in treating or preventing a cancer in a subject are provided.

### Pharmaceutical Compositions

In some embodiments, pharmaceutical compositions comprising an anti-TIGIT antibody for use in administering to a subject having a cancer are provided. In some embodiments, the anti-TIGIT antibody is as described in Section III above, e.g., an anti-TIGIT antibody having a binding affinity, activity, cross-reactivity, epitope recognition, and/or one or more CDR, VH, and/or VL sequences as disclosed in Section III above.

In some embodiments, an anti-TIGIT antibody and an immuno-oncology agent (e.g., a PD-1 pathway inhibitor as described herein) are formulated into pharmaceutical compositions, together or separately, as described herein. In some embodiments, the immuno-oncology agent is a PD-1 pathway inhibitor or a CTLA-4 pathway inhibitor. In some embodiments, the immuno-oncology agent is an agonist of a T cell coactivator. In some embodiments, the PD-1 pathway inhibitor is an anti-PD-1 antibody or anti-PD-L1 antibody, such as but not limited to pembrolizumab, nivolumab, durvalumab, pidilizumab, or atezolizumab.

Guidance for preparing formulations for use in the present invention is found in, for example, Remington: The Science and Practice of Pharmacy, 21^{st} Ed., 2006, supra; Martindale: The Complete Drug Reference, Sweetman, 2005, London: Pharmaceutical Press; Niazi, Handbook of Pharmaceutical Manufacturing Formulations, 2004, CRC Press; and Gibson, Pharmaceutical Preformulation and Formulation: A Practical Guide from Candidate Drug Selection to Commercial Dosage Form, 2001, Interpharm Press, which are hereby incorporated herein by reference. The pharmaceutical compositions described herein can be manufactured in a manner that is known to those of skill in the art, i.e., by means of conventional mixing, dissolving, granulating, dragee-making, emulsifying, encapsulating, entrapping or lyophilizing processes. The following methods and excipients are merely exemplary and are in no way limiting.

In some embodiments, an anti-TIGIT antibody (and optionally an immuno-oncology agent) is prepared for delivery in a sustained-release, controlled release, extended-release, timed-release or delayed-release formulation, for example, in semi-permeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

For oral administration, an anti-TIGIT antibody (and optionally an immuno-oncology agent) can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

The anti-TIGIT antibody (and optionally the immuno-oncology agent) can be formulated for parenteral administration by injection, e.g., by bolus injection or continuous infusion. For injection, the compound or compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, compounds can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

The anti-TIGIT antibody (and optionally the immuno-oncology agent) can be administered systemically by transmucosal or transdermal means. For transmucosal or transdermal administration, penetrants appropriate to the barrier to be permeated are used in the formulation. For topical administration, the agents are formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplary transdermal delivery formulations include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

In some embodiments, a pharmaceutical composition comprises an acceptable carrier and/or excipients. A pharmaceutically acceptable carrier includes any solvents, dispersion media, or coatings that are physiologically compatible and that preferably does not interfere with or otherwise inhibit the activity of the therapeutic agent. In some embodiments, the carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, 21st Edition, Philadelphia, Pa. Lippincott Williams & Wilkins, 2005. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

Dosages and desired drug concentration of pharmaceutical compositions of the disclosure may vary depending on the particular use envisioned. The determination of the appropriate dosage or route of administration is well within the skill of one in the art. Suitable dosages are also described in Section IV above.

### Kits

In some embodiments, kits for use in treating a subject having a cancer are provided. In some embodiments, the kit comprises:
an anti-TIGIT antibody; and
an immuno-oncology agent.

In some embodiments, anti-TIGIT antibody is as described in Section III above, e.g., an anti-TIGIT antibody having a binding affinity, activity, cross-reactivity, epitope recognition, and/or one or more CDR, VH, and/or VL sequences as disclosed in Section III above. In some embodiments, the immuno-oncology agent is a PD-1 pathway inhibitor or a CTLA-4 pathway inhibitor. In some embodiments, the immuno-oncology agent is an agonist of a T cell coactivator. In some embodiments, the PD-1 pathway inhibitor is an anti-PD-1 antibody or anti-PD-L1 antibody. In some embodiments, the immuno-oncology agent is pembrolizumab, nivolumab, durvalumab, pidilizumab, or atezolizumab.

In some embodiments, the kits can further comprise instructional materials containing directions (i.e., protocols) for the practice of the methods of this invention (e.g., instructions for using the kit for treating a cancer). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### VI. Examples

The following examples are offered to illustrate, but not to limit, the claimed invention.

### Example 1: Generation of Anti-TIGIT Antibodies

Fully human anti-TIGIT monoclonal antibodies were generated using yeast-based antibody presentation system (see, e.g., Xu et al, “Addressing polyspecificity of antibodies selected from an in vitro yeast presentation system: a FACS-based, high-throughput selection and analytical tool,” PEDS, 2013, 26:663-670; WO 2009/036379; WO 2010/105256; and WO 2012/009568). Eight naïve human synthetic yeast libraries each of ˜10⁹ diversity were screened. For the first two rounds of selection, a magnetic bead sorting technique utilizing the Miltenyi MACS system was performed, as previously described (see, e.g., Siegel et al, “High efficiency recovery and epitope-specific sorting of an scFv yeast display library,” J Immunol Methods, 2004, 286:141-153). Briefly, yeast cells (˜10¹⁰ cells/library) were incubated with 5 mL of 10 nM biotinylated Fc-fusion antigen for 30 minutes at 30° C. in wash buffer (phosphate-buffered saline (PBS)/0.1% bovine serum albumin (BSA)). After washing once with 40 mL ice-cold wash buffer, the cell pellet was resuspended in 20 mL wash buffer, and Streptavidin MicroBeads (500 μL) were added to the yeast and incubated for 15 minutes at 4° C. Next, the yeast were pelleted, resuspended in 20 ml wash buffer, and loaded onto a Miltenyi LS column. After the 20 mL were loaded, the column was washed 3 times with 3 mL wash buffer. The column was then removed from the magnetic field, and the yeast were eluted with 5 mL of growth media and then grown overnight. The following rounds of selection were performed using flow cytometry. Approximately 2×10⁷ yeast were pelleted, washed three times with wash buffer, and incubated at 30° C. with 10 nM Fe-fusion antigen and decreasing concentrations of biotinylated monomeric antigen (100 to 1 nM) under equilibrium conditions, 10 nM biotinylated Fe-fusion antigens or 100 nM monomeric antigens of different species in order to obtain species cross-reactivity, or with a polyspecificity depletion reagent (PSR) to remove non-specific antibodies from the selection. For the PSR depletion, the libraries were incubated with a 1:10 dilution of biotinylated PSR reagent as previously described (see, e.g., Xu et al, supra). Yeast were then washed twice with wash buffer and stained with LC-FITC (diluted 1:100) and either SA-633 (diluted 1:500) or EA-PE (extravidin-R-PE, diluted 1:50) secondary reagents for 15 minutes at 4° C. After washing twice with wash buffer, the cell pellets were resuspended in 0.3 mL, wash buffer and transferred to strainer-capped sort tubes. Sorting was performed using a FACS ARIA sorter (BD Biosciences) and sort gates were determined to select for antibodies with desired characteristics. Selection rounds were repeated until a population with all of the desired characteristics was obtained. After the final round of sorting, yeast were plated and individual colonies were picked for characterization.

Antigens included recombinant dimeric human TIGIT-Fc (Acro Biosystems TIT-H5254), monomeric human TIGIT (Sino Biological 10917-H08H), dimeric mouse TIGIT-Fc (R&D Systems, 7267-TG), and monomeric mouse TIGIT (Sino Biologics 50939-M08H).

Naïve campaign: 744 clones were sequenced yielding 345 unique clones (unique CDRH3). 18 VH germlines were represented in the clones.

Light chain batch diversification campaign: Heavy chain (VI) plasmids from an enriched binder pool from round six of the naïve discovery selections were extracted from the yeast via smash and grab, propagated in and subsequently purified from E. Coli, and then transformed into a light chain library with a diversity of 10⁷.

Selections were performed under essentially the same conditions as that for the naïve discovery. Briefly, one round of magnetic bead enrichment was followed by three rounds of selections by flow cytometry. In the magnetic bead enrichment round, 10 nM biotinylated Fc-fusion antigen was used. The first round on the flow cytometer consisted of a positive selection round using 100 nM biotinylated monovalent antigen. This was followed by a second round, which consisted of a negative selection round for PSR depletion. The final (third) round consisted of a positive selection round, in which the monovalent antigen was titrated at 100 nM, 10 nM, 1 nM. For all libraries, the yeasts from the 1 nM sorts from this third round were plated, and individual colonies were picked and characterized. In total, 728 clones were sequenced, yielding 350 unique HC/LC combinations (93 unique CDRH3s).

A total of 695 unique clones were identified between the naïve and the light chain batch shuffle campaigns.

### Example 2: Characterization of Anti-TIGIT Antibodies

65 clones were selected for production and further evaluation, representing 12 VH germlines and 9 VL germlines.

### Antibody Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over KappaSelect (GE Healthcare LifeSciences).

### Binding of Anti-TIGIT Antibodies to Recombinant Human and Mouse Protein

ForteBio affinity measurements were performed on an Octet RED384 generally as previously described (see, e.g., Estep et al., “High throughput solution-based measurement of antibody-antigen affinity and epitope binning,” Mabs, 2013, 5:270-278). Briefly, ForteBio affinity measurements were performed by loading IgGs on-line onto AHQ sensors. Sensors were equilibrated off-line in assay buffer for 30 minutes and then monitored on-line for 60 seconds for baseline establishment. Sensors with loaded IgGs were exposed to 100 nM antigen (dimeric Fc-fusion antigen or monomeric antigen) for 3 minutes, and afterwards were transferred to assay buffer for 3 minutes for off-rate measurement. All binding and dissociation kinetics were analyzed using the 1:1 binding model.

Of the 65 IgG clones, 43 had an affinity for TIGIT monomer of <100 nM. Of the 65 IgG clones, 34 cross-reacted with mouse TIGIT-Fc. Binding affinity for selected clones is shown in Table 1 below.

### Epitope Binning Ligand Competition Assay

Epitope binning/ligand blocking was performed using a standard sandwich format cross-blocking assay on the ForteBio Octet RED384 system. Control anti-target IgG was loaded onto AHQ sensors and unoccupied Fc-binding sites on the sensor were blocked with an irrelevant human IgG1 antibody. The sensors were then exposed to 100 nM target antigen followed by a second anti-target antibody or ligand (human CD155-Fc (Sino Biological, 10109-H02H)). Additional binding by the second antibody or ligand after antigen association indicates an unoccupied epitope (non-competitor), while no binding indicates epitope blocking (competitor or ligand blocking).

Four binning antibodies (not mutually exclusive) were used for bin assessment and five overlapping binning profiles were identified. 63 of the 65 anti-TIGIT antibodies competed with the ligand for binding to hTIGIT-Fc. Binning profiles and ligand competition results for selected clones are shown in Table 1 below.

TABLE 1
Epitope binning, ligand competition, and affinity data for selected anti-TIGIT clones

| | | CD155 | IgG KD Human | IgG KD Human TIGIT | IgG KD Mouse |
|---|---|---|---|---|---|
| Clone | Bin Code | Competition | TIGIT-Fc (M) | monomer (M) | TIGIT-Fc (M) |
| 2 | 1, 2, 3, 4 | Yes | 9.56E−10 | 1.01E−08 | 2.03E−09 |
| 3 | 1, 2, 3, 4 | Yes | 2.77E−09 | 7.36E−08 | 5.64E−09 |
| 5 | 1, 2, 3, 4 | Yes | 9.85E−10 | 1.41E−08 | 3.25E−09 |
| 13 | 1, 2, 3 | Yes | 5.43E−10 | 2.56E−09 | 1.16E−10 |
| 14 | 1, 2, 3 | Yes | 2.01E−09 | 5.87E−08 | 2.43E−09 |
| 16 | 1, 2, 3 | Yes | 6.90E−10 | 2.06E−09 | 1.05E−08 |
| 18 | 1, 2, 3 | Yes | 2.39E−09 | 5.08E−08 | 8.82E−09 |
| 21 | 1, 2, 3 | Yes | 5.85E−10 | 2.18E−09 | N.B. |
| 22 | 1, 2, 3 | Yes | 7.90E−10 | 1.38E−08 | 1.05E−08 |
| 25 | 1, 2, 3 | Yes | 6.20E−10 | 6.18E−10 | 1.10E−09 |
| 27 | 1, 2, 3 | Yes | 5.58E−10 | 2.32E−09 | N.B. |
| 54 | 1, 2, 3 | Yes | 6.89E−10 | 3.49E−09 | N.B. |

Notes:
N.B. = Non-Binder under the conditions of this assay
Bin code and CD155 competition data was generated on ForteBio Octet RED384 system using a standard sandwich format cross-blocking assay as described in Example 2.
KD affinity data was generated on ForteBio Octet RED384 system as described in Example 2.

### Binding of Anti-TIGIT Antibodies to Human, Mouse, and Cynomolgus Monkey TIGIT Overexpressed in HEK 293 Cells

HEK 293 cells were engineered to stably express high levels of human, mouse or cynomolgus monkey TIGIT by lentiviral transduction. Approximately 100,000 parental HEK 293 (TIGIT-negative) cells or HEK 293 cells overexpressing human, mouse or cynomolgus monkey were stained with 100 nM of each anti-TIGIT antibody for 5 minutes at room temperature. Cells were then washed twice with wash buffer and incubated with anti-human IgG conjugated to PE for 15 minutes on ice. Cells were then washed twice with wash buffer and analyzed by flow cytometry on a FACS Canto II instrument (BD Biosciences). Fold over background (FOB) was calculated as the median fluorescence intensity (MFI) of the anti-TIGIT clone bound to target-positive cells divided by the MFI of the anti-TIGIT clone bound to target-negative cells.

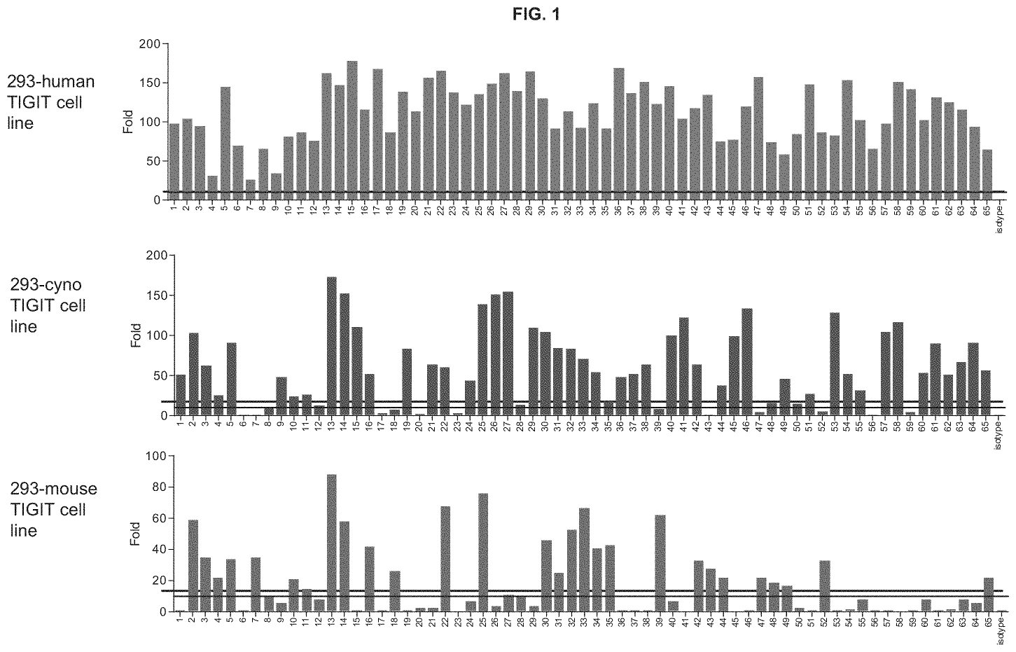

As shown in FIG. 1, all 65 antibodies showed specific binding to the 293-hTIGIT line (FOB>10, as indicated by the horizontal black line in the chart). 53 clones specifically bound the 293-cyTIGIT line while 31 clones specifically bound the 293-mTIGIT line.

### Polyspecificity Reagent (PSR) Assay

Assessment of binding to a polyspecificity reagent was conducted to determine specificity for TIGIT as previously described (see, e.g., Xu et al, supra). Briefly, biotinylated PSR reagent diluted 1:10 from stock was incubated with IgG-presenting yeast for 20 minutes on ice. Cells were washed and labeled with EA-PE (extravidin-R-PE) and read on a FACS analyzer. Scoring of polyspecific binding is on a 0 to 1 scale and is correlated to control IgGs with low, medium and high non-specific binding with a score of 0 indicating no binding and a score of 1 indicating very high non-specific binding.

62 of the 65 clones were scored as non-polyspecific binders with a PSR score of <0.10. Three clones scored as low polyspecific binders (PSR score 0.10-0.33).

### Hydrophobic Interaction Chromatography Assay

Hydrophobic interaction chromatography (HIC) was performed as described previously (Estep et al., supra). Briefly, 5 μg IgG samples were spiked in with a mobile phase A solution (1.8 M ammonium sulfate and 0.1 M sodium phosphate at pH 6.6) to achieve a final ammonium sulfate concentration of about 1 M before analysis. A Sepax Proteomix HIC butyl-NP5 column was used with a linear gradient of mobile phase A and mobile phase B solution (0.1 M sodium phosphate, pH 6.5) over 20 minutes at a flow rate of 1 mL/minute with UV absorbance monitoring at 280 nM.

Increased retention of antibodies on hydrophobic columns has been correlated with increased hydrophobicity and a propensity for poor expression, aggregation or precipitation during purification. Five of the 65 clones had high HIC retention time of >11.5 minutes, 10 clones had a medium HIC retention time of 10.5-11.5 minutes, and the remainder of the clones had low HIC retention times.

### Example 3: Binding of Anti-TIGIT Antibodies to Human, Mouse, and Cynomolgus Monkey TIGIT Endogenously Expressed on Primary T Cells

65 antibodies shown to be specific for human TIGIT recombinant protein and human TIGIT expressed on HEK 293 cells were evaluated for their ability to bind endogenous TIGIT on primary human peripheral blood T cells. Antibodies were also evaluated for cross reactivity to cynomolgus TIGIT on peripheral blood T cells and 35 of the 65 clones were evaluated for cross reactivity to mouse TIGIT on activated splenic T cells.

Human pan T cells were negatively isolated from leukapheresis product to 99% purity. 100,000 cells were stained at 4° C. for 30 minutes with 20 μg/mL of each anti-TIGIT antibody. The anti-TIGIT antibodies were detected with polyclonal goat anti-human IgG conjugated to PE (Jackson ImmunoResearch 109-116-098). Samples were analyzed on a CytoFLEX flow cytometer. Percent TIGIT+ of the FSC/SSC gated lymphocyte population was determined for each antibody using anti-human IgG-PE only staining to determine the threshold for positivity.

Cynomolgus white blood cells were isolated from whole blood by red blood cell lysis (eBioscience 00-4300). 200,000 cells were stained at 4° C. for 30 minutes with 20 μg/mL of each anti-TIGIT antibody. The anti-TIGIT antibodies were detected with polyclonal goat anti-human IgG adsorbed against monkey immunoglobulins conjugated to AlexaFluor647 (SouthernBiotech 2049-31) and T cells were identified by counterstaining with FITC-conjugated anti-CD3 clone SP34 (BD Pharmingen 556611). Samples were analyzed on a CytoFLEX flow cytometer. Percent TIGIT+ of the CD3+ population was determined for each antibody using anti-human IgG-PE only staining to determine the threshold for positivity.

BALB/c mouse T cells were isolated from spleens by negative selection (Stem Cell Technologies 19851A) to >99% purity. The cells were activated for 24 hours with plate bound anti-CD3 clone 145-2C11 (BioLegend 100302) to upregulate TIGIT. 200,000 activated cells were stained at 4° C. for 30 minutes with 20 μg/mL of each anti-TIGIT antibody (35 of 65 clones tested). The anti-TIGIT antibodies were detected with polyclonal goat anti-human IgG conjugated to PE (Jackson ImmunoResearch 109-116-098). Samples were analyzed on a FACSCalibur flow cytometer. Median fluorescence intensity of the FSC/SSC gated lymphocyte population was determined for each antibody.

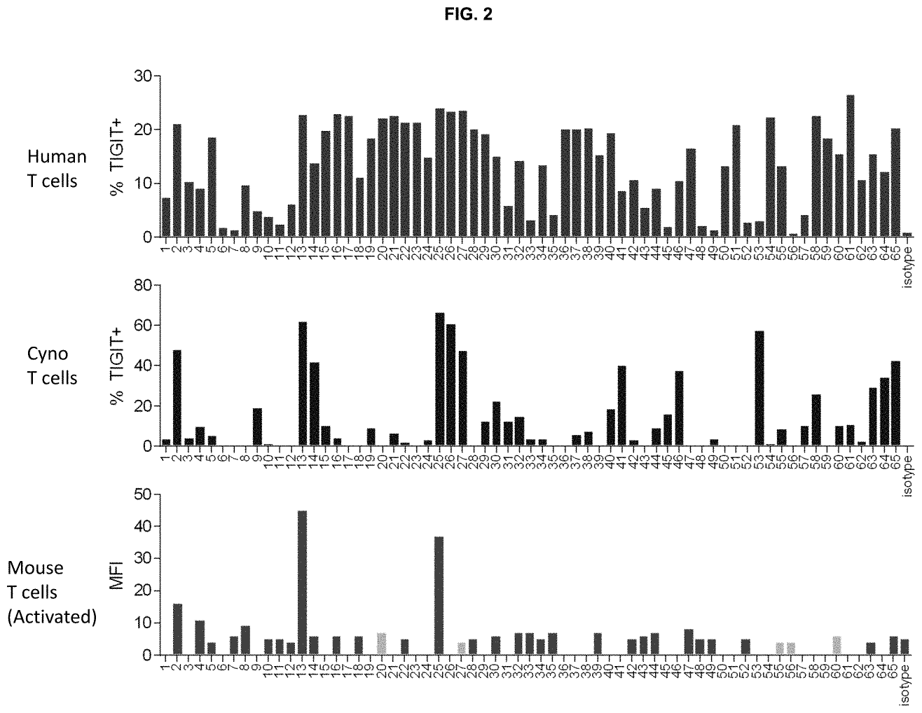

FIG. 2 shows binding of 65 anti-TIGIT antibody clones and an irrelevant isotype control antibody to primary human, cynomolgus monkey and mouse T cells. Clones 13 and 25 both showed strong binding to all three species of T cells.

### Titratable Binding of Anti-TIGIT Antibodies to Cell Surface Expressed TIGIT

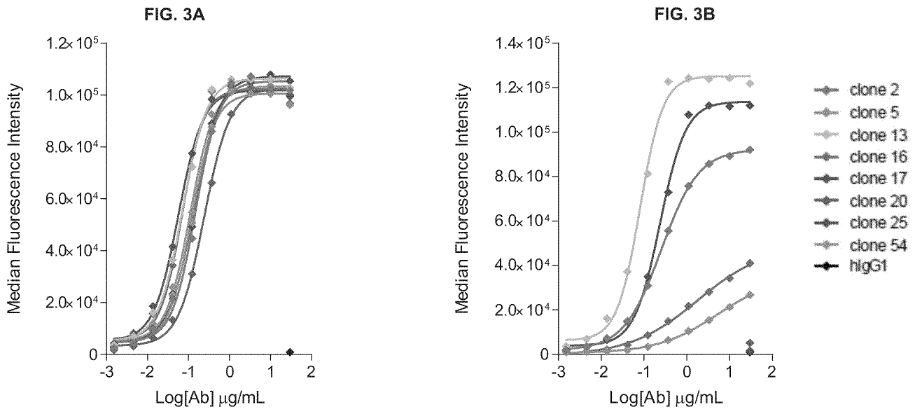

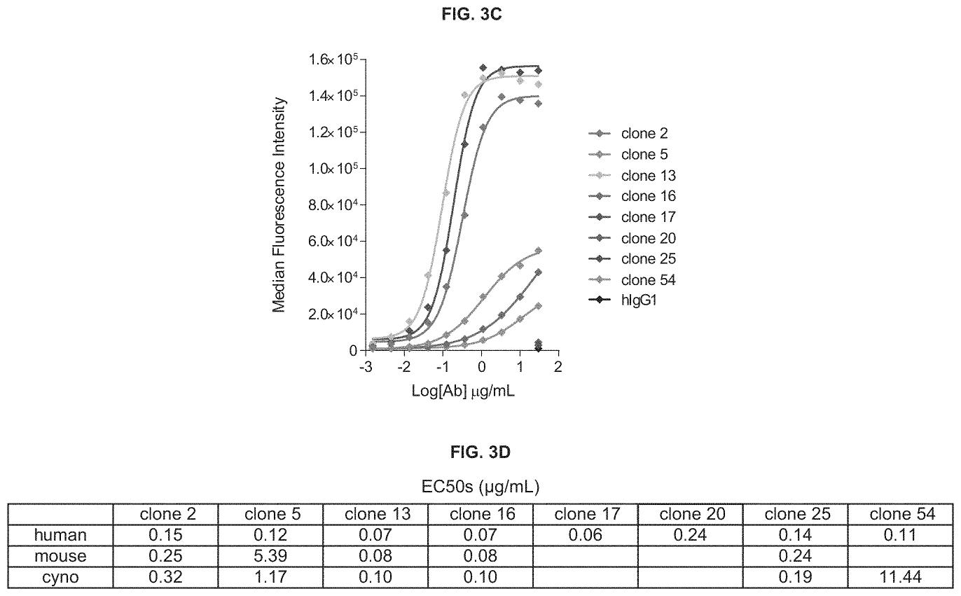

HEK 293 cells were engineered to stably express high levels of human, mouse or cynomolgus monkey TIGIT by lentiviral transduction. 200,000 293-TIGIT cells were stained at 4° C. for 30 minutes with a 10-point, 3-fold titration (30 to 0.002 μg/mL) of each anti-TIGIT antibody. The anti-TIGIT antibodies were detected with polyclonal goat anti-human IgG conjugated to PE (Jackson ImmunoResearch 109-116-098). Samples were analyzed on a CytoFLEX flow cytometer. Median fluorescence intensity of the FSC/SSC gated population was determined for each antibody concentration. NonLinear regression of Log(X) transformed data was used to generate EC50 values in GraphPad Prism 6. None of the anti-TIGIT antibodies showed binding to parental HEK 293 cells (TIGIT-) (data not shown). FIG. 3A-C shows the binding titration and FIG. 3D shows the EC50 of binding of eight anti-TIGIT antibody clones (clone 2, clone 5, clone 13, clone 16, clone 17, clone 20, clone 25, and clone 54) to human, cynomolgus monkey, and mouse TIGIT expressed on HEK 293 cells.

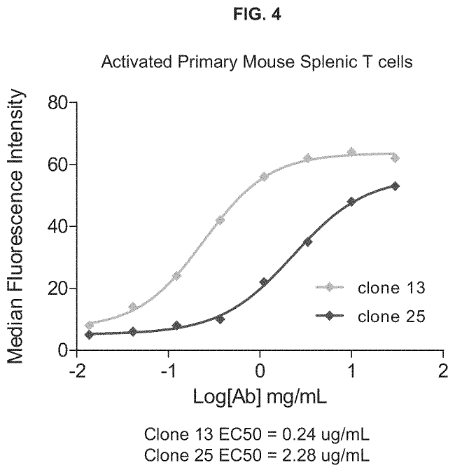

C57BL/6 mouse T cells were isolated from spleens by negative selection (Stem Cell Technologies 19851A) to >99% purity. The cells were activated for 24 hours with plate bound anti-CD3 clone 145-2C11 (BioLegend 100302) to upregulate TIGIT. 200,000 cells were stained at 4° C. for 30 minutes with an 8-point, 3-fold titration (30 to 0.014 μg/mL) of each anti-TIGIT antibody. The anti-TIGIT antibodies were detected with polyclonal goat anti-human IgG conjugated to PE (Jackson ImmunoResearch 109-116-098). Samples were analyzed on a FACSCalibur flow cytometer. Median fluorescence intensity of the FSC/SSC gated lymphocyte population was determined for each antibody. NonLinear regression of Log(X) transformed data was used to generate EC50 values in GraphPad Prism 6. FIG. 4 shows the binding titration and EC50 of binding of anti-TIGIT clones 13 and 25 to activated mouse splenic T cells.

### Example 4: Anti-TIGIT Antibodies Block Binding of CD155 and CD112 Ligand to Cell Surface-Expressed TIGIT

HEK 293 cells were engineered to stably express high levels of human or mouse TIGIT by lentiviral transduction. hCD155-Fc (Sino Biological 10109-H02H), hCD112-Fc (Sino Biological 10005-H02H) and mCD155-Fc (Sino Biological 50259-M03H) were conjugated to AlexaFluor647 (ThermoFisher A30009). 200,000 293-hTIGIT or 293-mTIGIT cells were co-incubated with 1 μg/mL CD155-Fc-AlexaFluor647 or 5 μg/mL CD112-Fc-AlexaFluor647 and a 12-point, 2-fold titration (10 to 0.005 μg/mL) of each anti-TIGIT antibody or an isotype control antibody. Samples were analyzed on a CytoFLEX flow cytometer. Median fluorescence intensity of the FSC/SSC gated population was determined for each antibody concentration. Percent blockade was calculated relative to the MFI of the no antibody control. NonLinear regression of Log(X) transformed data was performed in GraphPad Prism 6.

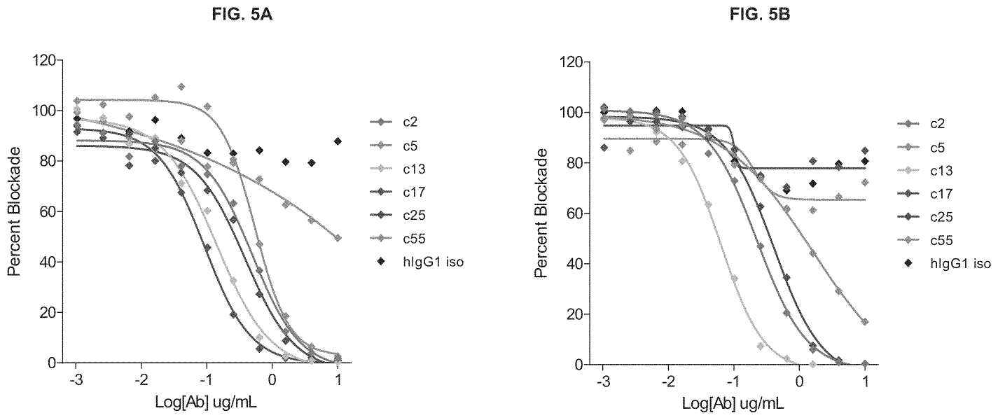

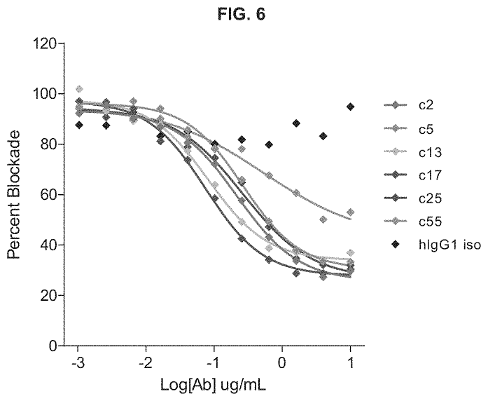

As shown in FIG. 5A-B, six anti-TIGIT antibody clones (clone 2, clone 5, clone 13, clone 17, clone 25, and clone 55) were tested, and five of the six clones (clone 2, clone 5, clone 13, clone 17, and clone 25) significantly blocked CD155 interaction with TIGIT expressed on HEK 293 cells for both human CD155/human TIGIT and for mouse CD155/mouse TIGIT. Clone 55 specifically binds human TIGIT but did not compete with hCD155-Fc for binding to hTIGIT-Fc in the ForteBio Octet ligand competition assay. Similarly, clone 55 did not efficiently block hCD155 interaction with the 293-hTIGIT cell line. Clone 2, clone 5, clone 13, clone 17, and clone 25 were also able to interrupt binding of human CD112 to human TIGIT. As observed for CD155, clone 55 was much less effective at blocking the CD112-TIGIT interaction. See, FIG. 6.

### Example 5: In Vitro Activity of Anti-TIGIT Antibodies in a TIGIT/CD155 Blockade Bioassay

The activity of anti-TIGIT antibodies can be functionally characterized using a TIGIT/CD155 blockade bioassay (e.g., TIGIT/CD155 Blockade Bioassay Kit, Promega Corp., Madison, Wis.), in which expression of a reporter gene is induced or enhanced when an antibody blocks TIGIT/CD155 interaction. The TIGIT/CD155 blockade bioassay comprises two cell types: an effector cell expressing TIGIT, CD226, and a TCR complex on the cell surface and containing a luciferase reporter gene; and an artificial antigen presenting cell that expresses CD155 and a TCR activator on the cell surface. In this bioassay, luciferase expression requires TCR engagement plus a co-stimulatory signal. The CD155-TIGIT interaction has higher affinity than the CD155-CD226 interaction, resulting in net inhibitory signaling and no luciferase expression. Blockade of the CD155-TIGIT interaction allows CD155-CD226 co-stimulation driving luciferase expression.

Jurkat effector cells expressing both TIGIT and CD226 were co-cultured with CHO-K1 artificial antigen presenting cells (aAPCs) expressing a TCR activator and CD155. The Jurkat effector cells contain a luciferase reporter gene driven by the IL-2 promoter. In the absence of blocking anti-TIGIT antibodies, CD155-TIGIT engagement leads to T cell co-inhibition and no IL-2 promoter activity. Upon addition of anti-TIGIT antibodies, CD155-TIGIT interaction is interrupted allowing CD155 to associate with CD226 to send a co-stimulatory signal and drive luciferase expression.

aAPCs were plated in 96-well plates and allowed to adhere overnight. The following day, 20 μg/mL of each anti-TIGIT antibody or an isotype control antibody and Jurkat effector cells were added to the plate. After a 6 hour incubation at 37° C., cells were lysed and luciferase substrate was added. Luciferase activity was quantified on a plate reader. Luciferase activity was calculated as a fold over the signal in the no antibody control.

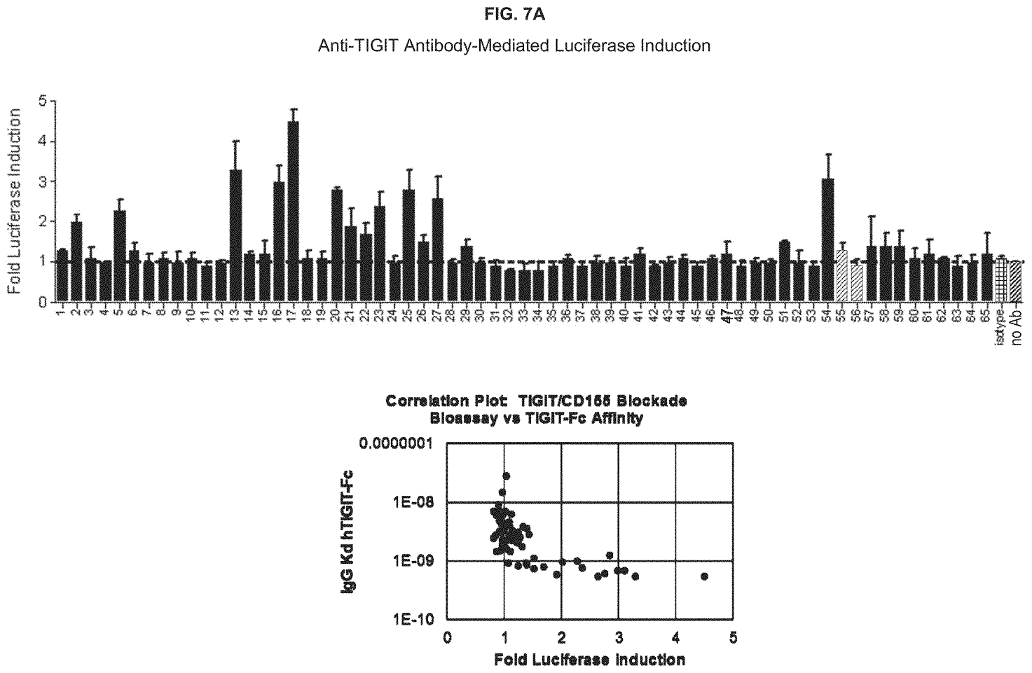

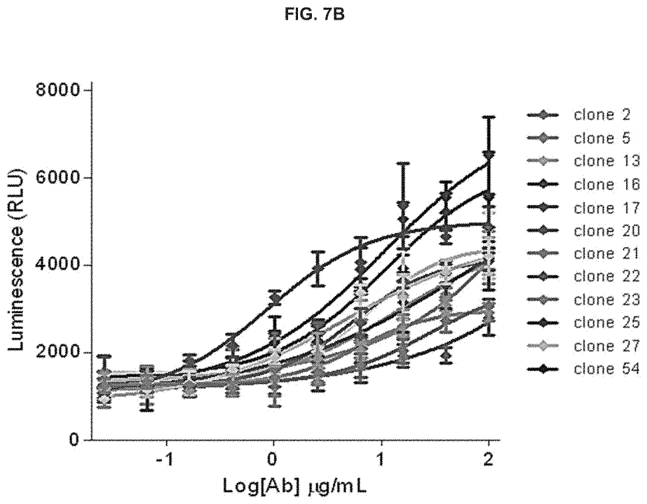

As shown in FIGS. 7A-7B, 12 anti-TIGIT antibody clones demonstrated functional blockade in this bioassay.

### Example 6: In Vitro Activity of Anti-TIGIT Antibodies in a TIGIT/PD-1 Combination Bioassay

The synergistic activity of anti-TIGIT antibodies in combination with anti-PD-1 agents (e.g., anti-PD-1 antibodies) can be functionally characterized using a TIGIT/PD-1 combination bioassay, in which expression of a reporter gene is enhanced when antibodies block both the TIGIT/CD155 interaction and the PD-1/PD-L1 interaction. The bioassay comprises two cell types: an effector cell expressing TIGIT, CD226, PD-1, and a TCR complex on the cell surface and containing a luciferase reporter gene; and an artificial antigen presenting cell that expresses CD155, PD-L1, and a TCR activator on the cell surface. In this bioassay, luciferase expression requires TCR engagement plus a co-stimulatory signal. The CD155-TIGIT interaction has higher affinity than the CD155-CD226 interaction, resulting in net inhibitory signaling and no luciferase expression. Additionally, binding of PD-L1 to PD-1 inhibits luciferase expression. Blockade of both the CD155-TIGIT interaction and the PD-1/PD-L1 interaction relieves the inhibition and allows CD155-CD226 co-stimulation driving luciferase expression.

Jurkat effector cells expressing PD-1, TIGIT and CD226 were co-cultured with CHO-K1 artificial antigen presenting cells (aAPCs) expressing a TCR activator, PD-L1 and CD155. The Jurkat effector cells contain a luciferase reporter gene driven by the IL-2 promoter. In the absence of blocking anti-TIGIT antibodies, PD-L1-PD-1 and CD155-TIGIT engagement leads to T cell co-inhibition and no IL-2 promoter activity. Upon addition of anti-PD-1 and anti-TIGIT antibodies, PD-L1-PD-1 interaction is blocked, relieving one co-inhibitory signal, and CD155-TIGIT interaction is interrupted, allowing CD155 to associate with CD226 to send a co-stimulatory signal and drive luciferase production.

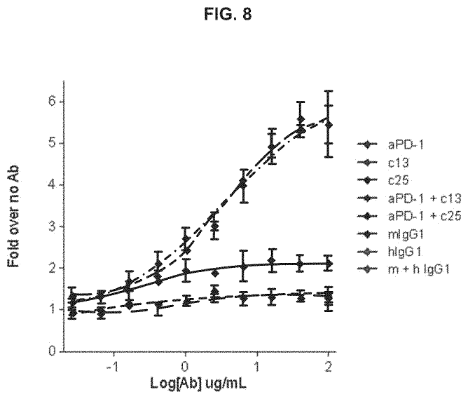

aAPCs were plated in 96-well plates and allowed to adhere overnight. The following day, a 10-point 2.5-fold titration (100 to 0.03 μg/mL) of each anti-TIGIT antibody alone, or anti-PD-1 antibody (clone EH12.2H7, BioLegend, San Diego, Calif.), or each anti-TIGIT antibody+anti-PD-1 antibody (11 ratio) and Jurkat effector cells were added to the plate. After a 6 hour incubation at 37° C., cells were lysed and luciferase substrate was added. Luciferase activity was quantified on a plate reader. Luciferase activity was calculated as a fold over the signal in the no antibody control. As shown in FIG. 8, neither anti-TIGIT nor anti-PD-1 alone led to dramatic Jurkat activation, however, the combination of either anti-TIGIT clone 13 or clone 25 with anti-PD-1 yielded strong activation.

### Example 7: In Vivo Activity of Anti-TIGIT Antibodies in a CT26 Syngeneic Tumor Model in BALB/c Mice

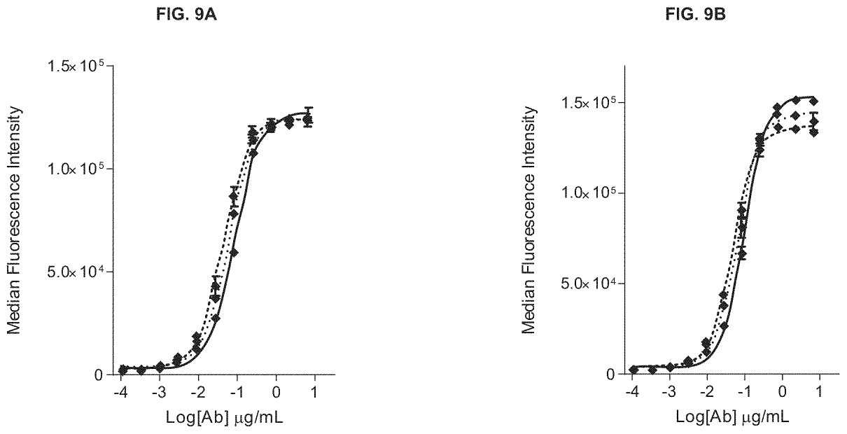

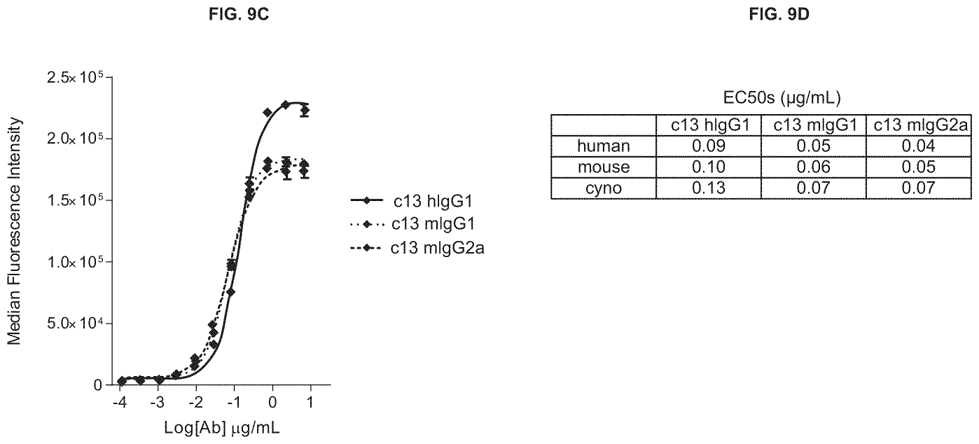

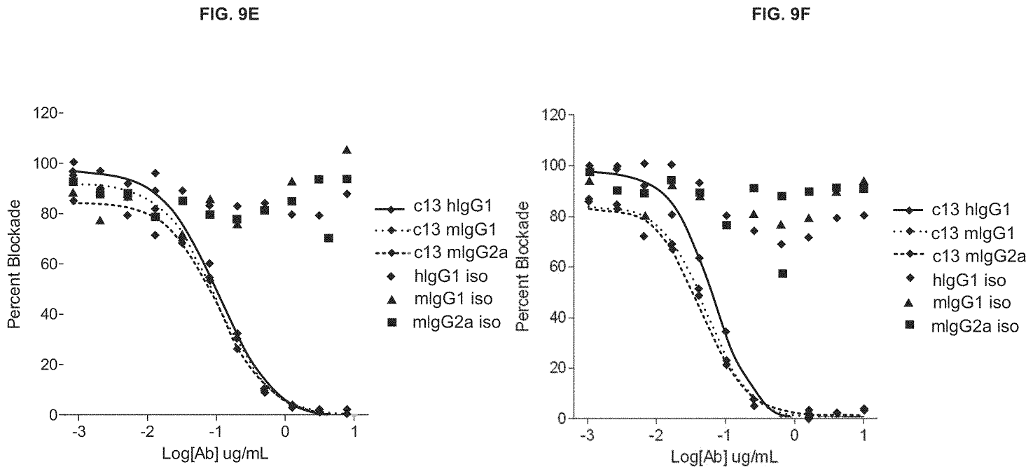

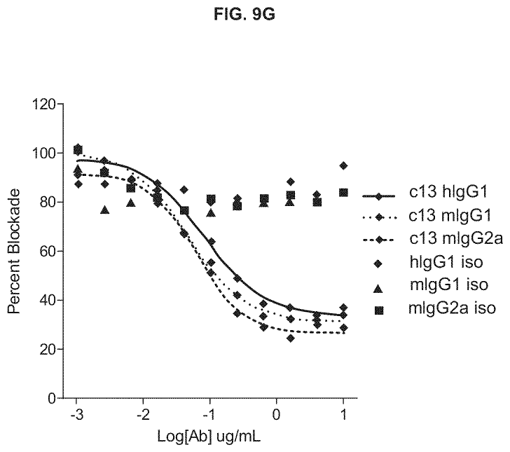

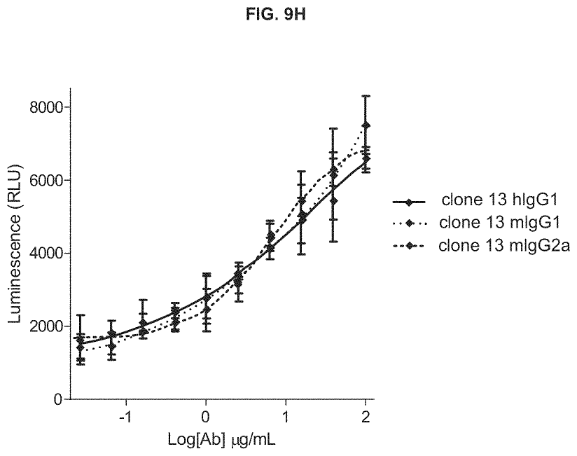

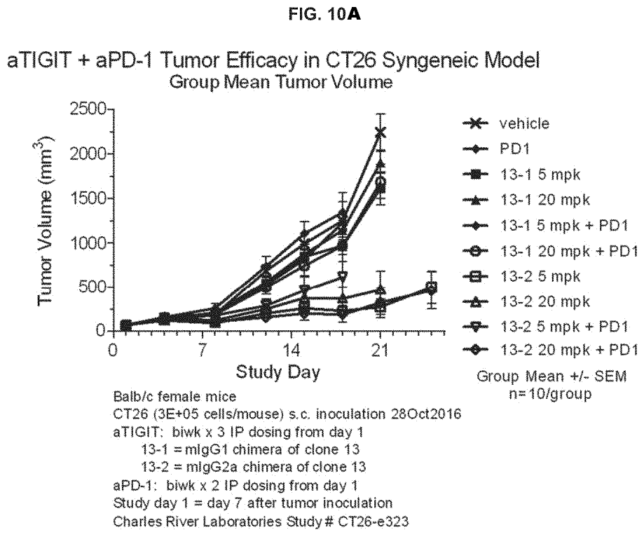

Based on affinity for murine TIGIT, anti-TIGIT clone 13 was chosen for evaluation in a murine syngeneic tumor model. Mouse IgG1 and mouse IgG2a chimeras of the parental fully human anti-TIGIT clone 13 were generated for in vivo experiments in order to address the question of whether Fc isotype has an effect on in vivo efficacy of antagonistic TIGIT antibodies. In vitro, the chimeric antibodies showed similar activity to the parental hIgG1 antibody with regards to (1) binding to human, mouse and cynomolgus monkey TIGIT, (2) blockade of CD155 and CD112 ligand binding to cell-surface expressed TIGIT and (3) activity in the CD155-TIGIT blockade bioassay. See FIG. 9A-9H.

8 week old BALB/c mice with an average body weight of 19 g were obtained from Charles River Laboratories. Mice were implanted subcutaneously with 300,000 CT26 colon carcinoma cells on the right lateral flank. Tumors were allowed to progress until the group average tumor volume was 72 mm³ (range of 48-88 mm³) on day 7 after tumor inoculation. Animals were allocated into 10 treatment groups of n=10 by pair match such that the group mean tumor volume was similar across all treatment groups. Tumor length and width were measured and tumor volume was calculated using the formula Volume (mm³)=0.5*Length*Width² where length is the longer dimension. Anti-TIGIT clone 13 mIgG1, anti-TIGIT clone 13 mIgG2a and anti-PD-1 clone RMP1-14 (BioXCell) were diluted to the appropriate concentration for dosing in sterile PBS. Sterile PBS was used as the vehicle control. TIGIT antibodies were dosed at 5 or 20 mg/kg via intraperitoneal injection twice weekly for 3 weeks (6 doses total). Anti-PD-1 antibody was dosed at 5 mg/kg via intraperitoneal injection twice weekly for 2 weeks (4 doses total). Dosing initiated on the day of allocation (study day 1). Tumor volume and body weight measurements were collected twice weekly until mice reached a tumor volume cutoff of 2000 mm³. None of the animals exhibited body weight loss relative to pre-dose weights indicating exceptional tolerability of all test agents.

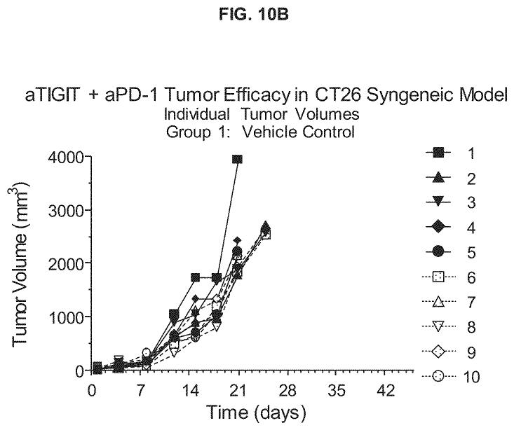

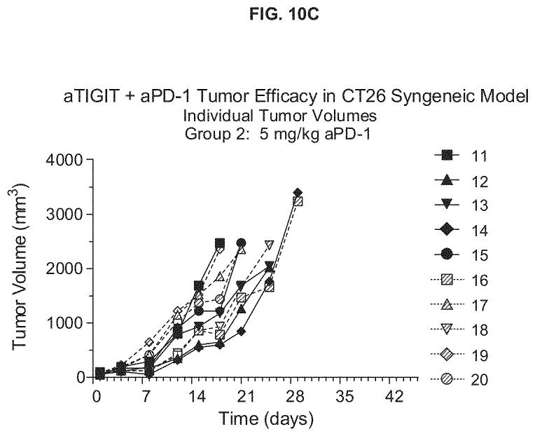

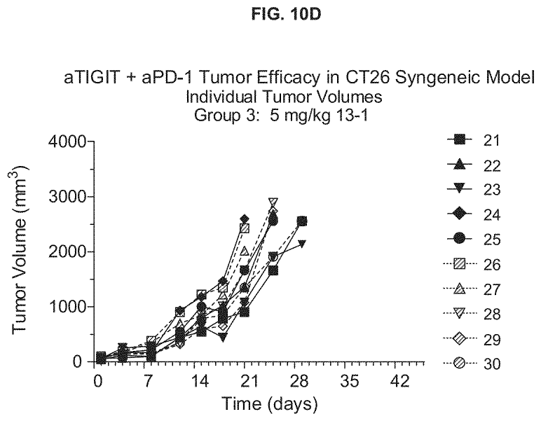

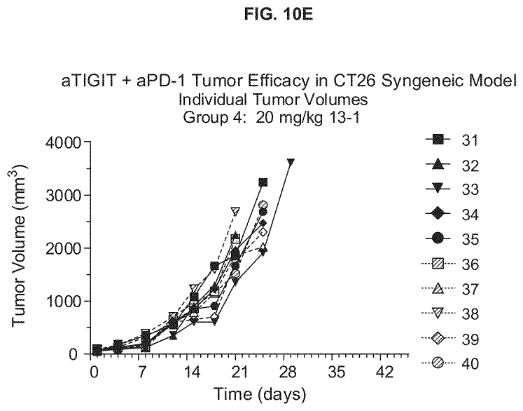

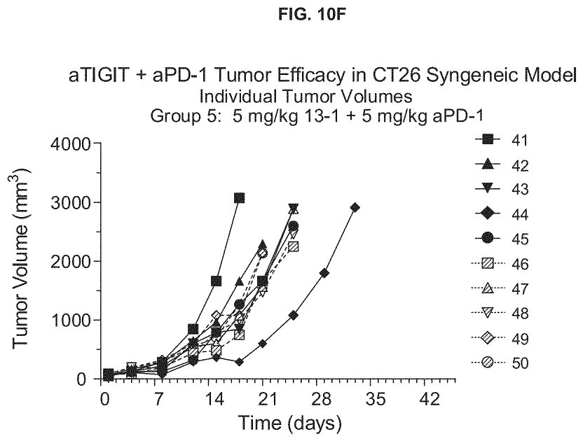

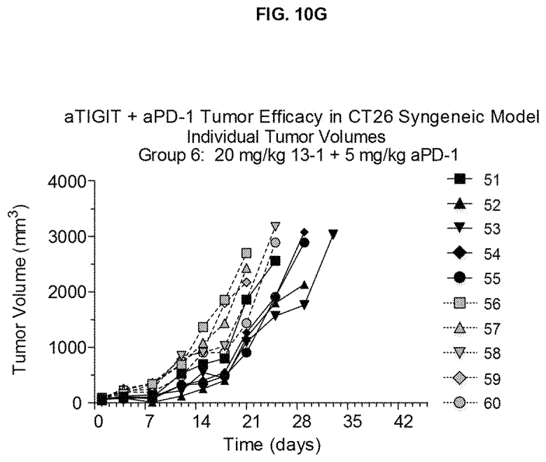

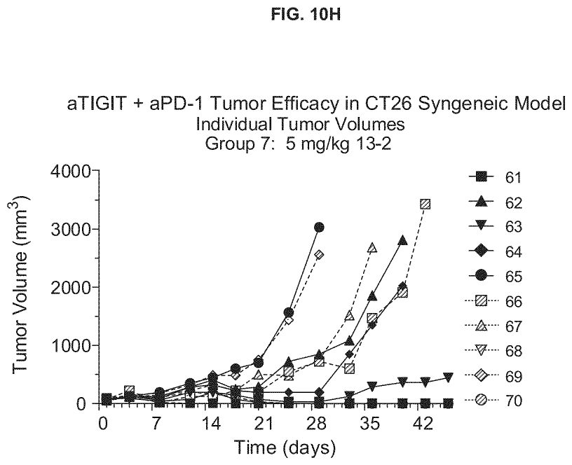

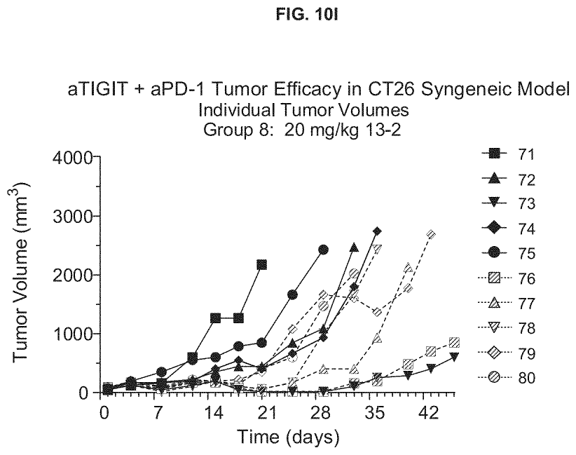

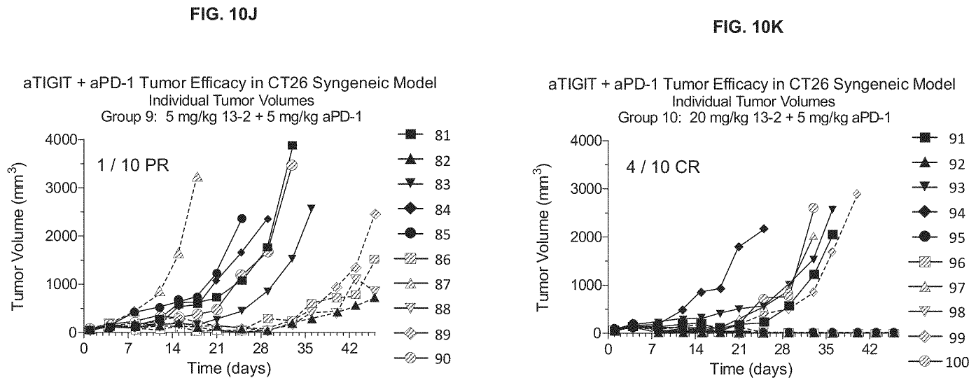

As shown in FIG. 10A, anti-mPD-1 alone did not have any effect on tumor progression. The mIgG1 anti-TIGIT chimera of clone 13 (“13-1”), which does not efficiently engage activating Fcgamma receptors, did not mediate any anti-tumor activity, either as a single agent or in combination with anti-PD-1. In contrast, the mIgG2a chimera of clone 13 (“13-2”), which is capable of binding activating Fcgamma receptors, slowed tumor progression (86.5% (5 mg/kg) or 74.4% (20 mg/kg) tumor growth inhibition on day 18). Three of ten animals in the 5 mg/kg 13-2 single agent group showed complete tumor regressions that were stable through the end of the study (study day 46). In the 20 mg/kg 13-2 single agent group, two of ten animals showed partial tumor regressions (defined as tumor volume <50% of initial volume for three consecutive measurements). FIG. 10A shows that the addition of anti-PD-1 to the mIgG2a clone 13 chimera (13-2) did not increase efficacy relative to 13-2 alone (day 18 tumor growth inhibition of 53.8% (5 mg/kg anti-TIGIT+5 mg/kg anti-PD1) vs 86.5% (5 mg/kg anti-TIGIT alone) and 89.6% (20 mg/kg anti-TIGIT+5 mg/kg anti-PD-1) vs 74.4% (20 mg/kg anti-TIGIT alone). Similar numbers of complete and partial responders were observed in the combination groups. See, e.g., FIGS. 10B-10K.

### Example 8: Antibody Optimization and Characterization of Optimized Antibodies

Antibody clones 2, 13, 16, and 25 from the primary discovery output were selected for further affinity maturation. Optimization of antibodies was performed via introducing diversities into the heavy chain variable region. Two cycles of optimization were applied to the above lineages. The first cycle was comprised of a CDRH1 and CDRH12 diversification approach, while in the second cycle a CDR13 mutagenesis approach was applied.

CDRH1 and CDRH2 approach: The CDRH3 of a single antibody was recombined into a premade library with CDRH1 and CDRH2 variants of a diversity of 1×10. Selections were then performed with one round of MACS and four rounds of FACS as described for the naïve discovery.

In the first FACS round, the libraries were sorted for 1 nM monomeric TIGIT binding. The second F ACS round was a PSR depletion round to reduce poly-specificity. The final two rounds were positive selection rounds using the parental Fab or IgG to pressure for high affinity. Fab/IgG pressure was performed as follows: antigen was incubated with 10 fold parental Fab or IgG and then incubated with the yeast libraries. Selections enriched for IgGs with better affinities than the parental Fab or IgG. Species cross-reactivity was checked in the last two rounds of FACS.

CDRH3 mutagenesis: Libraries were generated with CDRH3 diversification by randomizing positions in CDRH3. Selections were performed with one round of MACS and three rounds of FACS as described previously. PSR negative selections, species cross-reactivity, affinity pressure, and sorting was performed in order to obtain a population with the desired characteristics.

### MSD-SET K_{D} Measurements

Equilibrium affinity measurements were performed generally as previously described (Estep et al., supra). Briefly, solution equilibrium titrations (SET) were performed in PBS+0.1% IgG-Free BSA (PBSF) with biotinylated human TIGIT-His monomer held constant at 50 pM and incubated with 3- to 5-fold serial dilutions of antibody starting at around 5 nM. Antibodies (20 nM in PBS) were coated onto standard bind MSD-ECL plates overnight at 4° C. or at room temperature for 30 min. Plates were then blocked with 1% BSA for 30 min with shaking at 700 rpm, followed by three washes with wash buffer (PBSF+0.05% Tween 20). SET samples were applied and incubated on the plates for 150 s with shaking at 700 rpm followed by one wash. Antigen captured on a plate was detected with 250 ng/ml sulfotag-labeled streptavidin in PBSF by incubation on the plate for 3 min. The plates were washed three times with wash buffer and then read on the MSD Sector Imager 2400 instrument using 1×Read Buffer T with surfactant. The percent free antigen was plotted as a function of titrated antibody in Prism and fit to a quadratic equation to extract the K_{D}. To improve throughput, liquid handling robots were used throughout MSD-SET experiments, including SET sample preparation.

Binding of the optimized antibodies to His-tagged human TIGIT, cyno TIGIT-Fc, and mouse TIGIT-Fc was measured using the ForteBio system as described above. The optimized antibodies were also tested for ligand blocking in a CD155 ligand competition assay, and for binding to human TIGIT HEK, cyno TIGIT HEK, mouse TIGIT HEK, and parental HEK cell lines, as described above.

The affinity data and cell binding data for the affinity optimized antibodies is shown in Table 2 below.

TABLE 2
Affinity and Cell Binding Data for Affinity Optimzed Antibodies

| | ForteBio IgG | ForteBio IgG | ForteBio IgG | MSD IgG | Cell binding Human | Cell binding Cyno | Cell binding Mouse |
|---|---|---|---|---|---|---|---|
| | K_{D} Human | K_{D} Cyno | K_{D} Murine | K_{D} (M) | TIGIT HEK Cell | TIGIT HEK Cell | TIGIT HEK Cell |
| Clone | TIGIT-His | TIGIT-Fc | TIGIT-Fc | Human | (FOB Fold Over | (FOB Fold Over | (FOB Fold Over |
| Index | (M) Monovalent | (M) Avid | (M) Avid | TIGIT-His | Background) | Background) | Background) |
| 2 | 8.18E−09 | 1.34E−09 | 1.76E−09 | NA | 158 | 162 | 73 |
| 2C | 5.18E−10 | 9.84E−10 | 3.92E−10 | 1.60E−11 | 193 | 224 | 100 |
| 13 | 2.63E−09 | 1.04E−09 | 3.41E−10 | NA | 212 | 224 | 119 |
| 13A | 6.27E−10 | 1.12E−09 | 3.70E−10 | 2.50E−11 | 206 | 240 | 115 |
| 13B | 6.10E−10 | 1.05E−09 | 3.30E−10 | 5.30E−12 | 201 | 235 | 102 |
| 13C | 5.63E−10 | 1.07E−09 | 3.29E−10 | 8.60E−12 | 194 | 281 | 116 |
| 13D | 5.71E−10 | 1.16E−09 | 3.64E−10 | 5.00E−12 | 190 | 245 | 116 |
| 16 | 2.52E−09 | 4.67E−09 | 9.07E−09 | NA | 192 | 27 | 19 |
| 16C | 9.11E−10 | 4.25E−09 | 8.01E−10 | 6.30E−12 | 208 | 157 | 99 |
| 16D | 5.96E−10 | 1.15E−09 | 2.63E−09 | 1.30E−11 | 199 | 241 | 63 |
| 16E | 7.78E−10 | 1.36E−09 | 3.70E−09 | 1.10E−11 | 195 | 186 | 56 |
| 25 | 1.27E−09 | 1.50E−09 | 9.67E−10 | NA | 205 | 247 | 117 |
| 25A | 1.10E−09 | 1.64E−09 | 8.23E−10 | 1.80E−11 | 207 | 238 | 119 |
| 25B | 1.16E−09 | 1.40E−09 | 7.19E−10 | 2.20E−11 | 222 | 291 | 129 |
| 25C | 6.97E−10 | 1.24E−09 | 4.94E−10 | 5.60E−12 | 216 | 286 | 124 |
| 25D | 8.46E−10 | 1.18E−09 | 5.80E−10 | 2.70E−11 | 225 | 272 | 137 |
| 25E | 8.51E−10 | 1.18E−09 | 5.66E−10 | 1.30E−11 | 204 | 252 | 116 |

### Example 9: Epitope Mapping

The epitopes of two of the monoclonal antibodies disclosed herein, Clone 13 and Clone 25, were characterized by peptide array. To reconstruct epitopes of the target molecule a library of peptide based epitope mimics was synthesized using solid-phase Fmoc synthesis. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with Nhydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer).

Synthesis of structural mimics was performed using proprietary Chemically Linked Peptides on Scaffolds (CLIPS) technology (Pepscan). CLIPS technology allows to structure peptides into single loops, double-loops, triple loops, sheet-like folds, helix-like folds and combinations thereof. CLIPS templates are coupled to cysteine residues. The side-chains of multiple cysteines in the peptides are coupled to one or two CLIPS templates. For example, a 0.5 mM solution of the P2 CLIPS (2,6-bis(bromomethyl)pyridine) is dissolved in ammonium bicarbonate (20 mM, pH 7.8)/acetonitrile (1:3(v/v)). This solution is added onto the peptide arrays. The CLIPS template will bind to side-chains of two cysteines as present in the solid-phase bound peptides of the peptide-arrays (455 wells plate with 3 μl wells). The peptide arrays are gently shaken in the solution for 30 to 60 minutes while completely covered in solution. Finally, the peptide arrays are washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes. The T3 CLIPS carrying peptides were made in a similar way but with three cysteines.

Different sets of peptides were synthesized according to the following designs. Set 1 comprised a set of linear peptides having a length of 15 amino acids derived from the target sequence of human TIGIT with an offset of one residue. Set 2 comprised a set of linear peptides of Set 1, but with residues on positions 10 and 11 replaced by Ala. When a native Ala would occur on either position, it was replaced by Gly. Set 3 comprised a set of linear peptides of Set 1, which contained Cys residues. In this set, native Cys were replaced by Cys-acetamidomethyl (“Cys-acm”). Set 4 comprised a set of linear peptides having a length of 17 amino acids derived from the target sequence of human TIGIT with an offset of one residue. On positions 1 and 17 were Cys residues used to create looped mimics by means of mP2 CLIPS. Native Cys were replaced with Cys-acm. Set 6 comprised a set of linear peptides having a length of 22 amino acids derived from the target sequence of human TIGIT with an offset of one residue. Residues on positions 11 and 12 were replaced with “PG” motif, while Cys residues were placed on positions 1 and 22 to create a constrained mimic with mP2. Native Cys residues were replaced by Cys-acm. Set 7 contained a set of linear peptides having a length of 27 amino acids. On positions 1-11 and 17-27 were 11-mer peptide sequences derived from the target sequence and joined via “GGSGG” linker. Combinations were made based on the UniProt info on disulfide bridging for human TIGIT. Set 8 comprised a set of combinatorial peptides having a length of 33 amino acids. On positions 2-16 and 18-32 were 15-mer peptides derived from the target sequence of human TIGIT. On positions 1, 17 and 33 were Cys residues used to create discontinuous mimics by means of T3 CLIPS.

The binding of antibody to each of the synthesized peptides was tested in a pepscan-based ELISA. The peptide arrays were incubated with primary antibody solution (overnight at 4° C.). After washing, the peptide arrays were incubated with a 1/1000 dilution of a goat anti-human HRP conjugate (Southern Biotech) for one hour at 25° C. After washing, the peroxidase substrate 2,2′-azino-di-3-ethylbenzthiazoline sulfonate (ABTS) and 20 μl/ml of 3 percent H₂O₂ were added. After one hour, the color development was measured. The color development was quantified with a charge coupled device (CCD)—camera and an image processing system. The values obtained from the CCD camera range from 0 to 3000 mAU, similar to a standard 96-well plate ELISA-reader.

To verify the quality of the synthesized peptides, a separate set of positive and negative control peptides was synthesized in parallel. These were screened with commercial antibodies 3C9 and 57.9 (Posthumus et al., J. Virol., 1990, 64:3304-3309).

For Clone 13, when tested under high stringency conditions Clone 13 weakly bound discontinuous epitope mimics. The antibody was also tested under moderate stringency conditions and detectable binding of the antibody was observed. The highest signal intensities were recorded with discontinuous epitope mimics containing the core stretches ₆₈ICNADLGWHISPSFK₈₂, ₄₂ILQCHLSSTTAQV₅₄, ₁₀₈CIYHTYPDGTYTGRI₁₂₂. Additional, weaker binding was observed with peptides containing peptide stretch ₈₀SFKDRVAPGPG₉₀. Binding of the antibody to linear and simple conformational epitope mimics was generally lower and was only observed for motifs ₆₈ICNADLGWHISPSFK₈₂, ₁₀₈CIYHTYPDGTYTGRI₁₂₂ and ₈₀SFKDRVAPGPG₉₀.

For Clone 25, when tested under high stringency conditions Clone 25 detectably bound peptides from all sets. Strongest binding was observed with discontinuous epitope mimics. While binding to peptides containing residues within stretch ₆₈ICNADLGWHISPSFK₈₂ was also observed in other sets, binding to peptide stretch ₅₀TTAQVTQ₅₆ was only observed in combination with ₆₈ICNADLGWHISPSFK₈₂. Additional, weaker binding was also observed with peptides containing peptide stretch ₈₀SFKDRVAPGPGLGL₉₃.

Based on these epitope mapping results for Clone 13 and Clone 25, fine mapping of the epitopes of Clone 13 and Clone 25 was performed using the methods described above using the following sets of peptides. Set 1 comprised a library of single residue epitope mutants based on the sequence CILQ2HLSSTTAQVTQCI2NADLGWHISPSFKC. Residues ADHIQRY were subjected to replacement. Positions 1, 17, 19, 30 and 33 were not replaced. Native Cys residues were replaced by Cys-acm (denoted “2” throughout). Set 2 comprised a library of walking double Ala mutants derived from the sequence CILQ2HLSSTTAQVTQCI2NADLGWHISPSFKC. Positions 1, 17 and 33 were not replaced. Native Cys residues were replaced by Cys-acm. Set 3 comprised a library of single residue epitope mutants based on the sequence CKDRVAPGPGLGLTLQCI2NADLGWHISPSFKC. Residues ADHIQRY were used for the replacement. Positions 1, 2, 17, 19, 30 and 33 were not replaced. Set 4 comprised a library of walking double Ala mutants derived from sequence CKDRVAPGPGLGLTLQCI2NADLGWHISPSFKC. Positions 1, 17 and 33 were not replaced.

Clone 13 was tested with four series of discontinuous epitope mutants derived from peptides CILQ2HLSSTTAQVTQCI2NADLGWHISPSFKC and CKDRVAPGPGLGLTLQCI2NADLGWHISPSFKC under high and moderate stringency conditions. Data analysis indicated that in all instances, replacements of residues ₈₁FK₈₂ with either single residues or double Ala impaired binding of Clone 13. Single mutations of other residues within discontinuous epitope mimics did not have drastic effects on binding. On the contrary, double Ala epitope mutants displayed a more pronounced effect on binding when compared with the series of single residue mutants for the corresponding discontinuous mimics. It was also found that double Ala replacements of residues ₅₁TAQVT₅₅ within CILQ2HLSSTTAQVTQCI2NADLGWHISPSFKC notably impacted binding of Clone 13. Signal intensities recorded for Clone 13 with epitope mimics derived from sequence CKDRVAPGPGLGLTLQCI2NADLGWHISPSFKC were lower than those recorded with CILQ2HLSSTTAQVTQCI2NADLGWHISPSFKC. It was further found that that in addition to ₈₁FK₈₂ double Ala replacements of ₇₄GWHI₇₇ notably reduce binding of Clone 13. In addition, double Ala mutations within the stretch ₈₇PGPGLGL₉₃ somewhat weakened binding.

Clone 25 was tested on four series of discontinuous epitope mutants derived from peptides CILQ2HLSSTTAQVTQCI2NADLGWHISPSFKC and CKDRVAPGPGLGLTLQCI2NADLGWHISPSFKC under high and moderate stringency conditions. Analysis of data collected from individual sets of epitope mutants indicated that single or double replacements of residues ₈₁FK₈₂ drastically affected binding. Single residue replacements of other residues within CILQ2HLSSTTAQVTQCI2NADLGWHISPSFKC and CKDRVAPGPGLGLTLQCI2NADLGWHISPSFKC did not cause a notable decrease in signal intensities. A series of double walking Ala mutants displayed more pronounced effects on Clone 25 binding to the mimic. In addition to ₈₁FK₈₂, double Ala replacements of residues ₅₂AQ₅₃ and P79 also mildly affected binding of the antibody to the epitope mimic CILQ2HLSSTTAQVTQCI2NADLGWHISPSFKC. Analysis of binding of Clone 25 to double Ala mutant series derived from CKDRVAPGPGLGLTLQCI2NADLGWHISPSFKC again confirmed the importance of ₈₁FK₈₂, but also indicated that double Ala replacements of residues ₇₃LGW₇₅ and ₈₂KDRVA₈₆ moderately affected the binding.

In summary, for the monoclonal antibodies Clone 13 and Clone 25 it was found that residues ₈₁FK₈₂ were crucial for the binding of both antibodies to TIGIT epitope mimics. For Clone 13, the residues ₅₁TAQVT₅₅, ₇₄GWHI₇₇, and ₈₇PGPGLGL₉₃ were also found to contribute to binding. For Clone 25, the residues ₅₂AQ₅₃, ₇₃LGW₇₅, P79, and, ₈₂KDRVA₈₆ were also found to contribute to binding.

TABLE 3
Informal Sequence Listing

| | SEQ | |
|---|---|---|
| Name | ID NO | Sequence |
| Clone 2 VH Protein | 1 | EVQLVESGGGLVQPGGSLRLSCAASGFTFSDHYMDWVRQAPGKGLEWVG |
| | | RTRNKANSYTTEYAASVKGRFTISRDDSKNSLYLQMNSLKTEDTAVYYCA |
| | | RGQYYYGSSSRGYYYMDVWGQGTTVTVSS |
| Clone 2 VH DNA | 2 | GAGGTGCAGCTGGTGGAGTCTGGGGGAGGCTTGGTCCAGCCTGGAGGG |
| | | TCCCTGAGACTCTCCTGTGCAGCCTCTGGATTCACCTTCAGTGACCACTA |
| | | CATGGACTGGGTCCGCCAGGCTCCAGGGAAGGGGCTGGAGTGGGTTGG |
| | | CCGTACTAGAAACAAAGCTAACAGTTACACCACAGAATACGCCGCGTC |
| | | TGTGAAAGGCAGATTCACCATCTCAAGAGATGATTCAAAGAACTCACTG |
| | | TATCTGCAAATGAACAGCCTGAAAACCGAGGACACGGCGGTGTACTAC |
| | | TGCGCCAGAGGCCAGTACTACTACGGCAGCAGCAGCAGAGGTTACTAC |
| | | TACATGGACGTATGGGGCCAGGGAACAACCGTCACCGTCTCCTCA |
| Clone 2 VH FR1 | 3 | EVQLVESGGGLVQPGGSLRLSCAASG |
| Clone 2 VH CDR1 | 4 | FTFSDHYMD |
| Clone 2 VH FR2 | 5 | WVRQAPGKGLEWVG |
| Clone 2 VH CDR2 | 6 | RTRNKANSYTTEYAASVKG |
| Clone 2 VH FR3 | 7 | RFTISRDDSKNSLYLQMNSLKTEDTAVYYC |
| Clone 2 VH CDR3 | 8 | ARGQYYYGSSSRGYYYMDV |
| Clone 2 VH FR4 | 9 | WGQGTTVTVSS |
| Clones 2 and 2C VL | 10 | EIVLTQSPGTLSLSPGERATLSCRASQSVSSSYLAWYQQKPGQAPRLLIYGA |
| Protein | | SSRATGIPDRFSGSGSGTDFTLTISRLEPEDFAVYYCQQAVPSPLTFGGGTKV |
| | | EIK |
| Clone 2 VL DNA | 11 | GAAATTGTGTTGACGCAGTCTCCAGGCACCCTGTCTTTGTCTCCAGGGG |
| | | AAAGAGCCACCCTCTCCTGCAGGGCCAGTCAGAGTGTTAGCAGCAGCT |
| | | ACTTAGCCTGGTACCAGCAGAAACCTGGCCAGGCTCCCAGGCTCCTCAT |
| | | CTATGGTGCATCCAGCAGGGCCACTGGCATCCCAGACAGGTTCAGTGGC |
| | | AGTGGGTCTGGGACAGACTTCACTCTCACCATCAGCAGACTGGAGCCTG |
| | | AAGATTTTGCAGTGTATTACTGTCAGCAGGCCGTCCCCAGTCCTCTCACT |
| | | TTTGGCGGAGGGACCAAGGTTGAGATCAAA |
| Clone 2 VL FR1 | 12 | EIVLTQSPGTLSLSPGERATLSC |
| Clones 2 and 2C VL | 13 | RASQSVSSSYLA |
| CDR1 | | |
| Clone 2 VL FR2 | 14 | WYQQKPGQAPRLLIY |
| Clones 2 and 2C VL | 15 | GASSRAT |
| CDR2 | | |
| Clone 2 VL FR3 | 16 | GIPDRFSGSGSGTDFTLTISRLEPEDFAVYYC |
| Clones 2 and 2CVL | 17 | QQAVPSPLT |
| CDR3 | | |
| Clone 2 VL FR4 | 18 | FGGGTKVEIK |
| Clone 3 VH Protein | 19 | EVQLVESGGGLVQPGGSLRLSCAASGFTFSDHYMDWVRQAPGKGLEWVG |
| | | RTRNKANSYTTEYAASVKGRFTISRDDSKNSLYLQMNSLKTEDTAVYYCA |
| | | RGQYYYGSSSRGYYYMDVWGQGTTVTVSS |
| Clone 3 VH DNA | 20 | GAGGTGCAGCTGGTGGAGTCTGGGGGAGGCTTGGTCCAGCCTGGAGGG |
| | | TCCCTGAGACTCTCCTGTGCAGCCTCTGGATTCACCTTCAGTGACCACTA |
| | | CATGGACTGGGTCCGCCAGGCTCCAGGGAAGGGGCTGGAGTGGGTTGG |
| | | CCGTACTAGAAACAAAGCTAACAGTTACACCACAGAATACGCCGCGTC |
| | | TGTGAAAGGCAGATTCACCATCTCAAGAGATGATTCAAAGAACTCACTG |
| | | TATCTGCAAATGAACAGCCTGAAAACCGAGGACACGGCGGTGTACTAC |
| | | TGCGCCAGAGGCCAGTACTACTACGGCAGCAGCAGCAGAGGTTACTAC |
| | | TACATGGACGTATGGGGCCAGGGAACAACCGTCACCGTCTCCTCA |
| Clone 3 VH FR1 | 21 | EVQLVESGGGLVQPGGSLRLSCAASG |
| Clone 3 VH CDR1 | 22 | FTFSDHYMD |
| Clone 3 VH FR2 | 23 | WVRQAPGKGLEWVG |
| Clone 3 VH CDR2 | 24 | RTRNKANSYTTEYAASVKG |
| Clone 3 VH FR3 | 25 | RFTISRDDSKNSLYLQMNSLKTEDTAVYYC |
| Clone 3 VH CDR3 | 26 | ARGQYYYGSSSRGYYYMDV |
| Clone 3 VH FR4 | 27 | WGQGTTVTVSS |
| Clone 3 VL Protein | 28 | EIVLTQSPGTLSLSPGERATLSCRASQSVRSSYLAWYQQKPGQAPRLLIYGA |
| | | SSRATGIPDRFSGSGSGTDFTLTISRLEPEDFAVYYCQQVGPPLTFGGGTKVE |
| | | IK |
| Clone 3 VL DNA | 29 | GAAATTGTGTTGACGCAGTCTCCAGGCACCCTGTCTTTGTCTCCAGGGG |
| | | AAAGAGCCACCCTCTCCTGCAGGGCCAGTCAGAGTGTTAGGAGCAGCT |
| | | ACTTAGCCTGGTACCAGCAGAAACCTGGCCAGGCTCCCAGGCTCCTCAT |
| | | CTATGGTGCATCCAGCAGGGCCACTGGCATCCCAGACAGGTTCAGTGGC |
| | | AGTGGGTCTGGGACAGACTTCACTCTCACCATCAGCAGACTGGAGCCTG |
| | | AAGATTTTGCAGTGTATTACTGTCAGCAGGTCGGACCCCCCCTCACTTTT |
| | | GGCGGAGGGACCAAGGTTGAGATCAAA |
| Clone 3 VL FR1 | 30 | EIVLTQSPGTLSLSPGERATLSC |
| Clone 3 VL CDR1 | 31 | RASQSVRSSYLA |
| Clone 3 VL FR2 | 32 | WYQQKPGQAPRLLIY |
| Clone 3 VL CDR2 | 33 | GASSRAT |
| Clone 3 VL FR3 | 34 | GIPDRFSGSGSGTDFTLTISRLEPEDFAVYYC |
| Clone 3 VL CDR3 | 35 | QQVGPPLT |
| Clone 3 VL FR4 | 36 | FGGGTKVEIK |
| Clone 5 VH Protein | 37 | EVQLLESGGGLVQPGGSLRLSCAASGFTFSTYAMSWVRQAPGKGLEWVSA |
| | | ISGSGGSTYYADSVKGRFTISRDNSKNTLYLQMNSLRAEDTAVYYCAKGPR |
| | | YQDRAGMDVWGQGTTVTVSS |
| Clone 5 VH DNA | 38 | GAGGTGCAGCTGTTGGAGTCTGGGGGAGGCTTGGTACAGCCTGGGGGG |
| | | TCCCTGAGACTCTCCTGTGCAGCCTCTGGATTCACCTTTAGCACCTATGC |
| | | CATGAGCTGGGTCCGCCAGGCTCCAGGGAAGGGGCTGGAGTGGGTCTC |
| | | AGCTATTAGTGGTAGTGGTGGTAGCACATACTACGCAGACTCCGTGAAG |
| | | GGCCGGTTCACCATCTCCAGAGACAATTCCAAGAACACGCTGTATCTGC |
| | | AAATGAACAGCCTGAGAGCCGAGGACACGGCGGTGTACTACTGCGCCA |
| | | AGGGCCCCAGATACCAAGACAGGGCAGGAATGGACGTATGGGGCCAGG |
| | | GAACAACTGTCACCGTCTCCTCA |
| Clone 5 VH FR1 | 39 | EVQLLESGGGLVQPGGSLRLSCAASG |
| Clone 5 VH CDR1 | 40 | FTFSTYAMS |
| Clone 5 VH FR2 | 41 | WVRQAPGKGLEWVS |
| Clone 5 VH CDR2 | 42 | AISGSGGSTYYADSVKG |
| Clone 5 VH FR3 | 43 | RFTISRDNSKNTLYLQMNSLRAEDTAVYYC |
| Clone 5 VH CDR3 | 44 | AKGPRYQDRAGMDV |
| Clone 5 VH FR4 | 45 | WGQGTTVTVSS |
| Clone 5 VL Protein | 46 | DIQMTQSPSSLSASVGDRVTITCRASQSISSYLNWYQQKPGKAPKLLIYAAS |
| | | SLQSGVPSRFSGSGSGTDFTLTISSLQPEDFATYYCQQSLATPYTFGGGTKV |
| | | EIK |
| Clone 5 VL DNA | 47 | GACATCCAGATGACCCAGTCTCCATCCTCCCTGTCTGCATCTGTAGGAG |
| | | ACAGAGTCACCATCACTTGCCGGGCAAGTCAGAGCATTAGCAGCTATTT |
| | | AAATTGGTATCAGCAGAAACCAGGGAAAGCCCCTAAGCTCCTGATCTAT |
| | | GCTGCATCCAGTTTGCAAAGTGGGGTCCCATCAAGGTTCAGTGGCAGTG |
| | | GATCTGGGACAGATTTCACTCTCACCATCAGCAGTCTGCAACCTGAAGA |
| | | TTTTGCAACTTACTACTGTCAGCAAAGCCTCGCCACTCCTTACACTTTTG |
| | | GCGGAGGGACCAAGGTTGAGATCAAA |
| Clone 5 VL FR1 | 48 | DIQMTQSPSSLSASVGDRVTITC |
| Clone 5 VL CDR1 | 49 | RASQSISSYLN |
| Clone 5 VL FR2 | 50 | WYQQKPGKAPKLLIY |
| Clone 5 VL CDR2 | 51 | AASSLQS |
| Clone 5 VL FR3 | 52 | GVPSRFSGSGSGTDFTLTISSLQPEDFATYYC |
| Clone 5 VL CDR3 | 53 | QQSLATPYT |
| Clone 5 VL FR4 | 54 | FGGGTKVEIK |
| Clone 13 VH | 55 | QVQLVQSGAEVKKPGSSVKVSCKASGGTFSSYAISWVRQAPGQGLEWMG |
| Protein | | SIIPIFGTANYAQKFQGRVTITADESTSTAYMELSSLRSEDTAVYYCARGPSE |
| | | VGAILGYVWFDPWGQGTLVTVSS |
| Clone 13 VH DNA | 56 | CAGGTGCAGCTGGTGCAGTCTGGGGCTGAGGTGAAGAAGCCTGGGTCC |
| | | TCGGTGAAGGTCTCCTGCAAGGCTTCTGGAGGCACCTTCAGCAGCTATG |
| | | CTATCAGCTGGGTGCGACAGGCCCCTGGACAAGGGCTTGAGTGGATGG |
| | | GAAGCATCATCCCTATCTTTGGTACAGCAAACTACGCACAGAAGTTCCA |
| | | GGGCAGAGTCACGATTACCGCGGACGAATCCACGAGCACAGCCTACAT |
| | | GGAGCTGAGCAGCCTGAGATCTGAGGACACGGCGGTGTACTACTGCGC |
| | | CAGAGGCCCTTCTGAAGTAGGAGCAATACTCGGATATGTATGGTTCGAC |
| | | CCATGGGGACAGGGTACATTGGTCACCGTCTCCTCA |
| Clone 13 VH FR1 | 57 | QVQLVQSGAEVKKPGSSVKVSCKASG |
| Clone 13 VH CDR1 | 58 | GTFSSYAIS |
| Clone 13 VH FR2 | 59 | WVRQAPGQGLEWMG |
| Clone 13 VH CDR2 | 60 | SIIPIFGTANYAQKFQG |
| Clone 13 VH FR3 | 61 | RVTITADESTSTAYMELSSLRSEDTAVYYC |
| Clones 13 and 13A | 62 | ARGPSEVGAILGYVWFDP |
| VH CDR3 | | |
| Clone 13 VH FR4 | 63 | WGQGTLVTVSS |
| Clones 13, 13A, | 64 | DIVMTQSPLSLPVTPGEPASISCRSSQSLLHSNGYNYLDWYLQKPGQSPQLL |
| 13B, 13C, and 13D | | IYLGSNRASGVPDRFSGSGSGTDFTLKISRVEAEDVGVYYCMQARRIPITFG |
| VL Protein | | GGTKVEIK |
| Clone 13 VL DNA | 65 | GATATTGTGATGACTCAGTCTCCACTCTCCCTGCCCGTCACCCCTGGAG |
| | | AGCCGGCCTCCATCTCCTGCAGGTCTAGTCAGAGCCTCCTGCATAGTAA |
| | | TGGATACAACTATTTGGATTGGTACCTGCAGAAGCCAGGGCAGTCTCCA |
| | | CAGCTCCTGATCTATTTGGGTTCTAATCGGGCCTCCGGGGTCCCTGACA |
| | | GGTTCAGTGGCAGTGGATCAGGCACAGATTTTACACTGAAAATCAGCA |
| | | GAGTGGAGGCTGAGGATGTTGGGGTTTATTACTGCATGCAGGCAAGAC |
| | | GAATCCCTATCACTTTTGGCGGAGGGACCAAGGTTGAGATCAAA |
| Clone 13 VL FR1 | 66 | DIVMTQSPLSLPVTPGEPASISC |
| Clones 13, 13A, | 67 | RSSQSLLHSNGYNYLD |
| 13B, 13C, and 13D | | |
| VL CDR1 | | |
| Clone 13 VL FR2 | 68 | WYLQKPGQSPQLLIY |
| Clones 13, 13A, | 69 | LGSNRAS |
| 13B, 13C, and 13D | | |
| VL CDR2 | | |
| Clone 13 VL FR3 | 70 | GVPDRFSGSGSGTDFTLKISRVEAEDVGVYYC |
| Clones 13, 13A, | 71 | MQARRIPIT |
| 13B, 13C, and 13D | | |
| VL CDR3 | | |
| Clone 13 VL FR4 | 72 | FGGGTKVEIK |
| Clone 14 VH | 73 | QVQLVQSGAEVKKPGSSVKVSCKASGGTFSSYAISWVRQAPGQGLEWMG |
| Protein | | SIIPIFGTANYAQKFQGRVTITADESTSTAYMELSSLRSEDTAVYYCARGPSE |
| | | VGAILGYVWFDPWGQGTLVTVSS |
| Clone 14 VH DNA | 74 | CAGGTGCAGCTGGTGCAGTCTGGGGCTGAGGTGAAGAAGCCTGGGTCC |
| | | TCGGTGAAGGTCTCCTGCAAGGCTTCTGGAGGCACCTTCAGCAGCTATG |
| | | CTATCAGCTGGGTGCGACAGGCCCCTGGACAAGGGCTTGAGTGGATGG |
| | | GAAGCATCATCCCTATCTTTGGTACAGCAAACTACGCACAGAAGTTCCA |
| | | GGGCAGAGTCACGATTACCGCGGACGAATCCACGAGCACAGCCTACAT |
| | | GGAGCTGAGCAGCCTGAGATCTGAGGACACGGCGGTGTACTACTGCGC |
| | | CAGAGGCCCTTCTGAAGTAGGAGCAATACTCGGATATGTATGGTTCGAC |
| | | CCATGGGGACAGGGTACATTGGTCACCGTCTCCTCA |
| Clone 14 VH FR1 | 75 | QVQLVQSGAEVKKPGSSVKVSCKASG |
| Clone 14 VH CDR1 | 76 | GTFSSYAIS |
| Clone 14 VH FR2 | 77 | WVRQAPGQGLEWMG |
| Clone 14 VH CDR2 | 78 | SIIPIFGTANYAQKFQG |
| Clone 14 VH FR3 | 79 | RVTITADESTSTAYMELSSLRSEDTAVYYC |
| Clone 14 VH CDR3 | 80 | ARGPSEVGAILGYVWFDP |
| Clone 14 VH FR4 | 81 | WGQGTLVTVSS |
| Clone 14 VL Protein | 82 | DIVMTQSPLSLPVTPGEPASISCRSSQSLLHSNGYNYLDWYLQKPGQSPQLL |
| | | IYLGSNRASGVPDRFSGSGSGTDFTLKISRVEAEDVGVYYCMQAKRLPLTF |
| | | GGGTKVEIK |
| Clone 14 VL DNA | 83 | GATATTGTGATGACTCAGTCTCCACTCTCCCTGCCCGTCACCCCTGGAG |
| | | AGCCGGCCTCCATCTCCTGCAGGTCTAGTCAGAGCCTCCTGCATAGTAA |
| | | TGGATACAACTATTTGGATTGGTACCTGCAGAAGCCAGGGCAGTCTCCA |
| | | CAGCTCCTGATCTATTTGGGTTCTAATCGGGCCTCCGGGGTCCCTGACA |
| | | GGTTCAGTGGCAGTGGATCAGGCACAGATTTTACACTGAAAATCAGCA |
| | | GAGTGGAGGCTGAGGATGTTGGGGTTTATTACTGCATGCAGGCAAAAC |
| | | GACTCCCTCTCACTTTTGGCGGAGGGACCAAGGTTGAGATCAAA |
| Clone 14 VL FR1 | 84 | DIVMTQSPLSLPVTPGEPASISC |
| Clone 14 VL CDR1 | 85 | RSSQSLLHSNGYNYLD |
| Clone 14 VL FR2 | 86 | WYLQKPGQSPQLLIY |
| Clone 14 VL CDR2 | 87 | LGSNRAS |
| Clone 14 VL FR3 | 88 | GVPDRFSGSGSGTDFTLKISRVEAEDVGVYYC |
| Clone 14 VL CDR3 | 89 | MQAKRLPLT |
| Clone 14 VL FR4 | 90 | FGGGTKVEIK |
| Clone 16 VH | 91 | QVQLVQSGAEVKKPGSSVKVSCKASGGTFSSYAISWVRQAPGQGLEWMG |
| Protein | | GIIPIFGTASYAQKFQGRVTITADESTSTAYMELSSLRSEDTAVYYCARQST |
| | | WHKLYGTDVWGQGTTVTVSS |
| Clone 16 VH DNA | 92 | CAGGTGCAGCTGGTGCAGTCTGGGGCTGAGGTGAAGAAGCCTGGGTCC |
| | | TCGGTGAAGGTCTCCTGCAAGGCTTCTGGAGGCACCTTCAGCAGCTATG |
| | | CTATCAGCTGGGTGCGACAGGCCCCTGGACAAGGGCTTGAGTGGATGG |
| | | GAGGGATCATCCCTATCTTTGGTACAGCAAGCTACGCACAGAAGTTCCA |
| | | GGGCAGAGTCACGATTACCGCGGACGAATCCACGAGCACAGCCTACAT |
| | | GGAGCTGAGCAGCCTGAGATCTGAGGACACGGCGGTGTACTACTGCGC |
| | | AAGACAGAGCACCTGGCACAAATTGTACGGAACGGACGTATGGGGCCA |
| | | GGGAACAACTGTCACCGTCTCCTCA |
| Clone 16 VH FR1 | 93 | QVQLVQSGAEVKKPGSSVKVSCKASG |
| Clone 16 VH CDR1 | 94 | GTFSSYAIS |
| Clone 16 VH FR2 | 95 | WVRQAPGQGLEWMG |
| Clone 16 VH CDR2 | 96 | GIIPIFGTASYAQKFQG |
| Clone 16 VH FR3 | 97 | RVTITADESTSTAYMELSSLRSEDTAVYYC |
| Clone 16 VH CDR3 | 98 | ARQSTWHKLYGTDV |
| Clone 16 VH FR4 | 99 | WGQGTTVTVSS |
| Clones 16, 16C, | 100 | DIQMTQSPSSVSASVGDRVTITCRASQGISSWLAWYQQKPGKAPKLLIYAA |
| 16D, and 16E VL | | SSLQSGVPSRFSGSGSGTDFTLTISSLQPEDFATYYCQQGDSLPPTFGGGTKV |
| Protein | | EIK |
| Clone 16 VL DNA | 101 | GACATCCAGATGACCCAGTCTCCATCTTCCGTGTCTGCATCTGTAGGAG |
| | | ACAGAGTCACCATCACTTGTCGGGCGAGTCAGGGTATTAGCAGCTGGTT |
| | | AGCCTGGTATCAGCAGAAACCAGGGAAAGCCCCTAAGCTCCTGATCTAT |
| | | GCTGCATCCAGTTTGCAAAGTGGGGTCCCATCAAGGTTCAGCGGCAGTG |
| | | GATCTGGGACAGATTTCACTCTCACCATCAGCAGCCTGCAGCCTGAAGA |
| | | TTTTGCAACTTATTACTGTCAGCAGGGAGACAGTCTCCCTCCTACTTTTG |
| | | GCGGAGGGACCAAGGTTGAGATCAAA |
| Clone 16 VL FR1 | 102 | DIQMTQSPSSVSASVGDRVTITC |
| Clones 16, 16C, | 103 | RASQGISSWLA |
| 16D, and 16E VL | | |
| CDR1 | | |
| Clone 16 VL FR2 | 104 | WYQQKPGKAPKLLIY |
| Clones 16, 16C, | 105 | AASSLQS |
| 16D, and 16E VL | | |
| CDR2 | | |
| Clone 16 VL FR3 | 106 | GVPSRFSGSGSGTDFTLTISSLQPEDFATYYC |
| Clones 16, 16C, | 107 | QQGDSLPPT |
| 16D, and 16E VL | | |
| CDR3 | | |
| Clone 16 VL FR4 | 108 | FGGGTKVEIK |
| Clone 18 VH | 109 | QVQLVQSGAEVKKPGASVKVSCKASGYTFTSYYMSWVRQAPGQGLEWM |
| Protein | | GIINPSGGSTSYAQKFQGRVTMTRDTSTSTVYMELSSLRSEDTAVYYCARV |
| | | RYGYADGMDVWGQGTTVTVSS |
| Clone 18 VH DNA | 110 | CAGGTGCAGCTGGTGCAGTCTGGGGCTGAGGTGAAGAAGCCTGGGGCC |
| | | TCAGTGAAGGTTTCCTGCAAGGCATCTGGATACACCTTCACCAGCTACT |
| | | ATATGTCATGGGTGCGACAGGCCCCTGGACAAGGGCTTGAGTGGATGG |
| | | GAATAATCAACCCTAGTGGTGGTAGCACAAGCTACGCACAGAAGTTCC |
| | | AGGGCAGAGTCACCATGACCAGGGACACGTCCACGAGCACAGTCTACA |
| | | TGGAGCTGAGCAGCCTGAGATCTGAGGACACGGCGGTGTACTACTGCG |
| | | CCAGAGTGAGGTACGGATACGCAGACGGAATGGACGTATGGGGCCAGG |
| | | GAACAACTGTCACCGTCTCCTCA |
| Clone 18 VH FR1 | 111 | QVQLVQSGAEVKKPGASVKVSCKASG |
| Clone 18 VH CDR1 | 112 | YTFTSYYMS |
| Clone 18 VH FR2 | 113 | WVRQAPGQGLEWMG |
| Clone 18 VH CDR2 | 114 | IINPSGGSTSYAQKFQG |
| Clone 18 VH FR3 | 115 | RVTMTRDTSTSTVYMELSSLRSEDTAVYYC |
| Clone 18 VH CDR3 | 116 | ARVRYGYADGMDV |
| Clone 18 VH FR4 | 117 | WGQGTTVTVSS |
| Clone 18 VL Protein | 118 | DIQMTQSPSSLSASVGDRVTITCRASQSISSYLNWYQQKPGKAPKLLIYGAS |
| | | SLQSGVPSRFSGSGSGTDFTLTISSLQPEDFATYYCQQVYHLPFTFGGGTKV |
| | | EIK |
| Clone 18 VL DNA | 119 | GACATCCAGATGACCCAGTCTCCATCCTCCCTGTCTGCATCTGTAGGAG |
| | | ACAGAGTCACCATCACTTGCCGGGCAAGTCAGAGCATTAGCAGCTATTT |
| | | AAATTGGTATCAGCAGAAACCAGGGAAAGCCCCTAAGCTCCTGATCTAT |
| | | GGTGCATCCAGTTTGCAAAGTGGGGTCCCATCAAGGTTCAGTGGCAGTG |
| | | GATCTGGGACAGATTTCACTCTCACCATCAGCAGTCTGCAACCTGAAGA |
| | | TTTTGCAACTTACTACTGTCAGCAAGTATACCACCTCCCTTTCACTTTTG |
| | | GCGGAGGGACCAAGGTTGAGATCAAA |
| Clone 18 VL FR1 | 120 | DIQMTQSPSSLSASVGDRVTITC |
| Clone 18 VL CDR1 | 121 | RASQSISSYLN |
| Clone 18 VL FR2 | 122 | WYQQKPGKAPKLLIY |
| Clone 18 VL CDR2 | 123 | GASSLQS |
| Clone 18 VL FR3 | 124 | GVPSRFSGSGSGTDFTLTISSLQPEDFATYYC |
| Clone 18 VL CDR3 | 125 | QQVYHLPFT |
| Clone 18 VL FR4 | 126 | FGGGTKVEIK |
| Clone 21 VH | 127 | QLQLQESGPGLVKPSETLSLTCTVSGGSISSSSYYWGWIRQPPGKGLEWIGSI |
| Protein | | YYSGSTYYNPSLKSRVTISVDTSKNQFSLKLSSVTAADTAVYYCARDPLYQ |
| | | DAPFDYWGQGTLVTVSS |
| Clone 21 VH DNA | 128 | CAGCTGCAGCTGCAGGAGTCGGGCCCAGGACTGGTGAAGCCTTCGGAG |
| | | ACCCTGTCCCTCACCTGCACTGTCTCTGGTGGCTCCATCAGCAGTAGTA |
| | | GTTACTACTGGGGCTGGATCCGCCAGCCCCCAGGGAAGGGGCTGGAGT |
| | | GGATTGGGAGTATCTATTATAGTGGGAGCACCTACTACAACCCGTCCCT |
| | | CAAGAGTCGAGTCACCATATCCGTAGACACGTCCAAGAACCAGTTCTCC |
| | | CTGAAGCTGAGTTCTGTGACCGCCGCAGACACGGCGGTGTACTACTGCG |
| | | CCAGAGATCCTTTGTACCAAGACGCTCCCTTCGACTATTGGGGACAGGG |
| | | TACATTGGTCACCGTCTCCTCA |
| Clone 21 VH FR1 | 129 | QLQLQESGPGLVKPSETLSLTCTVSG |
| Clone 21 VH CDR1 | 130 | GSISSSSYYWG |
| Clone 21 VH FR2 | 131 | WIRQPPGKGLEWIG |
| Clone 21 VH CDR2 | 132 | SIYYSGSTYYNPSLKS |
| Clone 21 VH FR3 | 133 | RVTISVDTSKNQFSLKLSSVTAADTAVYYC |
| Clone 21 VH CDR3 | 134 | ARDPLYQDAPFDY |
| Clone 21 VH FR4 | 135 | WGQGTLVTVSS |
| Clone 21 VL Protein | 136 | EIVLTQSPATLSLSPGERATLSCRASQSVSSYLAWYQQKPGQAPRLLIYDAS |
| | | NRATGIPARFSGSGSGTDFTLTISSLEPEDFAVYYCQQRANFPTFGGGTKVEI |
| | | K |
| Clone 21 VL DNA | 137 | GAAATTGTGTTGACACAGTCTCCAGCCACCCTGTCTTTGTCTCCAGGGG |
| | | AAAGAGCCACCCTCTCCTGCAGGGCCAGTCAGAGTGTTAGCAGCTACTT |
| | | AGCCTGGTACCAACAGAAACCTGGCCAGGCTCCCAGGCTCCTCATCTAT |
| | | GATGCATCCAACAGGGCCACTGGCATCCCAGCCAGGTTCAGTGGCAGT |
| | | GGGTCTGGGACAGACTTCACTCTCACCATCAGCAGCCTAGAGCCTGAAG |
| | | ATTTTGCAGTTTATTACTGTCAGCAGAGAGCCAACTTCCCTACTTTTGGC |
| | | GGAGGGACCAAGGTTGAGATCAAA |
| Clone 21 VL FR1 | 138 | EIVLTQSPATLSLSPGERATLSC |
| Clone 21 VL CDR1 | 139 | RASQSVSSYLA |
| Clone 21 VL FR2 | 140 | WYQQKPGQAPRLLIY |
| Clone 21 VL CDR2 | 141 | DASNRAT |
| Clone 21 VL FR3 | 142 | GIPARFSGSGSGTDFTLTISSLEPEDFAVYYC |
| Clone 21 VL CDR3 | 143 | QQRANFPT |
| Clone 21 VL FR4 | 144 | FGGGTKVEIK |
| Clone 22 VH | 145 | QVQLQESGPGLVKPSETLSLTCAVSGYSISSGYYWAWIRQPPGKGLEWIGSI |
| Protein | | YHSGSTYYNPSLKSRVTISVDTSKNQFSLKLSSVTAADTAVYYCARQGYYY |
| | | GSSGSVDFDLWGRGTLVTVSS |
| Clone 22 VH DNA | 146 | CAGGTGCAGCTGCAGGAGTCGGGCCCAGGACTGGTGAAGCCTTCGGAG |
| | | ACCCTGTCCCTCACCTGCGCTGTCTCTGGTTACTCCATCAGCAGTGGTTA |
| | | CTACTGGGCTTGGATCCGGCAGCCCCCAGGGAAGGGGCTGGAGTGGAT |
| | | TGGGAGTATCTATCATAGTGGGAGCACCTACTACAACCCGTCCCTCAAG |
| | | AGTCGAGTCACCATATCAGTAGACACGTCCAAGAACCAGTTCTCCCTGA |
| | | AGCTGAGTTCTGTGACCGCCGCAGACACGGCGGTGTACTACTGCGCCAG |
| | | GCAGGGATACTACTACGGCAGCAGCGGCAGTGTAGACTTCGACCTATG |
| | | GGGGAGAGGTACCTTGGTCACCGTCTCCTCA |
| Clone 22 VH FR1 | 147 | QVQLQESGPGLVKPSETLSLTCAVSG |
| Clone 22 VH CDR1 | 148 | YSISSGYYWA |
| Clone 22 VH FR2 | 149 | WIRQPPGKGLEWIG |
| Clone 22 VH CDR2 | 150 | SIYHSGSTYYNPSLKS |
| Clone 22 VH FR3 | 151 | RVTISVDTSKNQFSLKLSSVTAADTAVYYC |
| Clone 22 VH CDR3 | 152 | ARQGYYYGSSGSVDFDL |
| Clone 22 VH FR4 | 153 | WGRGTLVTVSS |
| Clone 22 VL Protein | 154 | DIQMTQSPSSVSASVGDRVTITCRASQGISSWLAWYQQKPGKAPKLLIYAA |
| | | SNLQSGVPSRFSGSGSGTDFTLTISSLQPEDFATYYCQQANSLPPWTFGGGT |
| | | KVEIK |
| Clone 22 VL DNA | 155 | GACATCCAGATGACCCAGTCTCCATCTTCCGTGTCTGCATCTGTAGGAG |
| | | ACAGAGTCACCATCACTTGTCGGGCGAGTCAGGGTATTAGCAGCTGGTT |
| | | AGCCTGGTATCAGCAGAAACCAGGGAAAGCCCCTAAGCTCCTGATCTAT |
| | | GCTGCATCCAATTTGCAAAGTGGGGTCCCATCAAGGTTCAGCGGCAGTG |
| | | GATCTGGGACAGATTTCACTCTCACCATCAGCAGCCTGCAGCCTGAAGA |
| | | TTTTGCAACTTATTACTGTCAACAGGCAAATAGTCTCCCTCCTTGGACTT |
| | | TTGGCGGAGGGACCAAGGTTGAGATCAAA |
| Clone 22 VL FR1 | 156 | DIQMTQSPSSVSASVGDRVTITC |
| Clone 22 VL CDR1 | 157 | RASQGISSWLA |
| Clone 22 VL FR2 | 158 | WYQQKPGKAPKLLIY |
| Clone 22 VL CDR2 | 159 | AASNLQS |
| Clone 22 VL FR3 | 160 | GVPSRFSGSGSGTDFTLTISSLQPEDFATYYC |
| Clone 22 VL CDR3 | 161 | QQANSLPPWT |
| Clone 22 VL FR4 | 162 | FGGGTKVEIK |
| Clone 25 VH | 163 | QVQLVQSGAEVKKPGASVKVSCKASGYTFTSYAISWVRQAPGQGLEWMG |
| Protein | | WISAYNGNTNYAQKLQGRVTMTTDTSTSTAYMELRSLRSDDTAVYYCAR |
| | | DLSSFWSGDVLGAFDIWGQGTMVTVSS |
| Clone 25 VH DNA | 164 | CAGGTTCAGCTGGTGCAGTCTGGAGCTGAGGTGAAGAAGCCTGGGGCC |
| | | TCAGTGAAGGTCTCCTGCAAGGCTTCTGGTTACACCTTTACCAGCTATG |
| | | CCATCAGCTGGGTGCGACAGGCCCCTGGACAAGGGCTTGAGTGGATGG |
| | | GATGGATCAGCGCTTACAATGGTAACACAAACTATGCACAGAAGCTCC |
| | | AGGGCAGAGTCACCATGACCACAGACACATCCACGAGCACAGCCTACA |
| | | TGGAGCTGAGGAGCCTGAGATCTGACGACACGGCGGTGTACTACTGCG |
| | | CAAGGGATTTGTCTAGCTTCTGGAGCGGAGACGTGTTAGGAGCCTTCGA |
| | | CATATGGGGTCAGGGTACAATGGTCACCGTCTCCTCA |
| Clone 25 VH FR1 | 165 | QVQLVQSGAEVKKPGASVKVSCKASG |
| Clones 25 and 25A | 166 | YTFTSYAIS |
| VH CDR1 | | |
| Clone 25 VH FR2 | 167 | WVRQAPGQGLEWMG |
| Clones 25 and 25E | 168 | WISAYNGNTNYAQKLQG |
| VH CDR2 | | |
| Clone 25 VH FR3 | 169 | RVTMTTDTSTSTAYMELRSLRSDDTAVYYC |
| Clones 25, 25A, and | 170 | ARDLSSFWSGDVLGAFDI |
| 25B VH CDR3 | | |
| Clone 25 VH FR4 | 171 | WGQGTMVTVSS |
| Clones 25, 25A, | 172 | DIQMTQSPSSLSASVGDRVTITCRASQSISSYLNWYQQKPGKAPKLLIYAAS |
| 25B, 25C, 25D, and | | SLQSGVPSRFSGSGSGTDFTLTISSLQPEDFATYYCQQSVPPRTFGGGTKVEI |
| 25E VL Protein | | K |
| Clone 25 VL DNA | 173 | GACATCCAGATGACCCAGTCTCCATCCTCCCTGTCTGCATCTGTAGGAG |
| | | ACAGAGTCACCATCACTTGCCGGGCAAGTCAGAGCATTAGCAGCTATTT |
| | | AAATTGGTATCAGCAGAAACCAGGGAAAGCCCCTAAGCTCCTGATCTAT |
| | | GCTGCATCCAGTTTGCAAAGTGGGGTCCCATCAAGGTTCAGTGGCAGTG |
| | | GATCTGGGACAGATTTCACTCTCACCATCAGCAGTCTGCAACCTGAAGA |
| | | TTTTGCAACTTACTACTGTCAGCAAAGCGTCCCCCCCAGGACTTTTGGC |
| | | GGAGGGACCAAGGTTGAGATCAAA |
| Clone 25 VL FR1 | 174 | DIQMTQSPSSLSASVGDRVTITC |
| Clones 25, 25A, | 175 | RASQSISSYLN |
| 25B, 25C, 25D, and | | |
| 25E VL CDR1 | | |
| Clone 25 VL FR2 | 176 | WYQQKPGKAPKLLIY |
| Clones 25, 25A, | 177 | AASSLQS |
| 25B, 25C, 25D, and | | |
| 25E VL CDR2 | | |
| Clone 25 VL FR3 | 178 | GVPSRFSGSGSGTDFTLTISSLQPEDFATYYC |
| Clones 25, 25A, | 179 | QQSVPPRT |
| 25B, 25C, 25D, and | | |
| 25E VL CDR3 | | |
| Clone 25 VL FR4 | 180 | FGGGTKVEIK |
| Clone 27 VH | 181 | QVQLVQSGAEVKKPGASVKVSCKASGYTFTSYAISWVRQAPGQGLEWMG |
| Protein | | WISAYNGNTNYAQKLQGRVTMTTDTSTSTAYMELRSLRSDDTAVYYCAR |
| | | DLSSFWSGDVLGAFDIWGQGTMVTVSS |
| Clone 27 VH DNA | 182 | CAGGTTCAGCTGGTGCAGTCTGGAGCTGAGGTGAAGAAGCCTGGGGCC |
| | | TCAGTGAAGGTCTCCTGCAAGGCTTCTGGTTACACCTTTACCAGCTATG |
| | | CCATCAGCTGGGTGCGACAGGCCCCTGGACAAGGGCTTGAGTGGATGG |
| | | GATGGATCAGCGCTTACAATGGTAACACAAACTATGCACAGAAGCTCC |
| | | AGGGCAGAGTCACCATGACCACAGACACATCCACGAGCACAGCCTACA |
| | | TGGAGCTGAGGAGCCTGAGATCTGACGACACGGCGGTGTACTACTGCG |
| | | CAAGGGATTTGTCTAGCTTCTGGAGCGGAGACGTGTTAGGAGCCTTCGA |
| | | CATATGGGGTCAGGGTACAATGGTCACCGTCTCCTCA |
| Clone 27 VH FR1 | 183 | QVQLVQSGAEVKKPGASVKVSCKASG |
| Clone 27 VH CDR1 | 184 | YTFTSYAIS |
| Clone 27 VH FR2 | 185 | WVRQAPGQGLEWMG |
| Clone 27 VH CDR2 | 186 | WISAYNGNTNYAQKLQG |
| Clone 27 VH FR3 | 187 | RVTMTTDTSTSTAYMELRSLRSDDTAVYYC |
| Clone 27 VH CDR3 | 188 | ARDLSSFWSGDVLGAFDI |
| Clone 27 VH FR4 | 189 | WGQGTMVTVSS |
| Clone 27 VL Protein | 190 | EIVMTQSPATLSVSPGERATLSCRASQSVSSNLAWYQQKPGQAPRLLIYGA |
| | | STRATGIPARFSGSGSGTEFTLTISSLQSEDFAVYYCQQHANHITFGGGTKVE |
| | | IK |
| Clone 27 VL DNA | 191 | GAAATAGTGATGACGCAGTCTCCAGCCACCCTGTCTGTGTCTCCAGGGG |
| | | AAAGAGCCACCCTCTCCTGCAGGGCCAGTCAGAGTGTTAGCAGCAACTT |
| | | AGCCTGGTACCAGCAGAAACCTGGCCAGGCTCCCAGGCTCCTCATCTAT |
| | | GGTGCATCCACCAGGGCCACTGGTATCCCAGCCAGGTTCAGTGGCAGTG |
| | | GGTCTGGGACAGAGTTCACTCTCACCATCAGCAGCCTGCAGTCTGAAGA |
| | | TTTTGCAGTTTATTACTGTCAGCAGCACGCCAATCACATCACTTTTGGCG |
| | | GAGGGACCAAGGTTGAGATCAAA |
| Clone 27 VL FR1 | 192 | EIVMTQSPATLSVSPGERATLSC |
| Clone 27 VL CDR1 | 193 | RASQSVSSNLA |
| Clone 27 VL FR2 | 194 | WYQQKPGQAPRLLIY |
| Clone 27 VL CDR2 | 195 | GASTRAT |
| Clone 27 VL FR3 | 196 | GIPARFSGSGSGTEFTLTISSLQSEDFAVYYC |
| Clone 27 VL CDR3 | 197 | QQHANHIT |
| Clone 27 VL FR4 | 198 | FGGGTKVEIK |
| Clone 54 VH | 199 | QVQLVQSGAEVKKPGASVKVSCKASGYTFTSYYMHWVRQAPGQGLEWM |
| Protein | | GIINPSGGSTSYAQKFQGRVTMTRDTSTSTVYMELSSLRSEDTAVYYCARA |
| | | SDSYGVGLYYGMDVWGQGTTVTVSS |
| Clone 54 VH DNA | 200 | CAGGTGCAGCTGGTGCAGTCTGGGGCTGAGGTGAAGAAGCCTGGGGCC |
| | | TCAGTGAAGGTTTCCTGCAAGGCATCTGGATACACCTTCACCAGCTACT |
| | | ATATGCACTGGGTGCGACAGGCCCCTGGACAAGGGCTTGAGTGGATGG |
| | | GAATAATCAACCCTAGTGGTGGTAGCACAAGCTACGCACAGAAGTTCC |
| | | AGGGCAGAGTCACCATGACCAGGGACACGTCCACGAGCACAGTCTACA |
| | | TGGAGCTGAGCAGCCTGAGATCTGAGGACACGGCGGTGTACTACTGCG |
| | | CTAGGGCATCTGACTCCTACGGAGTGGGCCTCTACTACGGAATGGACGT |
| | | ATGGGGCCAGGGAACAACTGTCACCGTCTCCTCA |
| Clone 54 VH FR1 | 201 | QVQLVQSGAEVKKPGASVKVSCKASG |
| Clone 54 VH CDR1 | 202 | YTFTSYYMH |
| Clone 54 VH FR2 | 203 | WVRQAPGQGLEWMG |
| Clone 54 VH CDR2 | 204 | HNPSGGSTSYAQKFQG |
| Clone 54 VH FR3 | 205 | RVTMTRDTSTSTVYMELSSLRSEDTAVYYC |
| Clone 54 VH CDR3 | 206 | ARASDSYGVGLYYGMDV |
| Clone 54 VH FR4 | 207 | WGQGTTVTVSS |
| Clone 54 VL Protein | 208 | EIVLTQSPGTLSLSPGERATLSCRASQSVRSSYLAWYQQKPGQAPRLLIYGA |
| | | SSRATGIPDRFSGSGSGTDFTLTISRLEPEDFAVYYCQQYYVSPLTFGGGTK |
| | | VEIK |
| Clone 54 VL DNA | 209 | GAAATTGTGTTGACGCAGTCTCCAGGCACCCTGTCTTTGTCTCCAGGGG |
| | | AAAGAGCCACCCTCTCCTGCAGGGCCAGTCAGAGTGTTAGGAGCAGCT |
| | | ACTTAGCCTGGTACCAGCAGAAACCTGGCCAGGCTCCCAGGCTCCTCAT |
| | | CTATGGTGCATCCAGCAGGGCCACTGGCATCCCAGACAGGTTCAGTGGC |
| | | AGTGGGTCTGGGACAGACTTCACTCTCACCATCAGCAGACTGGAGCCTG |
| | | AAGATTTTGCAGTGTATTACTGTCAGCAGTACTACGTCAGTCCTCTCACT |
| | | TTTGGCGGAGGGACCAAGGTTGAGATCAAA |
| Clone 54 VL FR1 | 210 | EIVLTQSPGTLSLSPGERATLSC |
| Clone 54 VL CDR1 | 211 | RASQSVRSSYLA |
| Clone 54 VL FR2 | 212 | WYQQKPGQAPRLLIY |
| Clone 54 VL CDR2 | 213 | GASSRAT |
| Clone 54 VL FR3 | 214 | GIPDRFSGSGSGTDFTLTISRLEPEDFAVYYC |
| Clone 54 VL CDR3 | 215 | QQYYVSPLT |
| Clone 54 VL FR4 | 216 | FGGGTKVEIK |
| Human TIGIT | 217 | CGTCCTATCTGCAGTCGGCTACTTTCAGTGGCAGAAGAGGCCACATCTG |
| cDNA sequence | | CTTCCTGTAGGCCCTCTGGGCAGAAGCATGCGCTGGTGTCTCCTCCTGA |
| (GenBank | | TCTGGGCCCAGGGGCTGAGGCAGGCTCCCCTCGCCTCAGGAATGATGAC |
| Accession No. | | AGGCACAATAGAAACAACGGGGAACATTTCTGCAGAGAAAGGTGGCTC |
| NM_173799.3) | | TATCATCTTACAATGTCACCTCTCCTCCACCACGGCACAAGTGACCCAG |
| | | GTCAACTGGGAGCAGCAGGACCAGCTTCTGGCCATTTGTAATGCTGACT |
| | | TGGGGTGGCACATCTCCCCATCCTTCAAGGATCGAGTGGCCCCAGGTCC |
| | | CGGCCTGGGCCTCACCCTCCAGTCGCTGACCGTGAACGATACAGGGGA |
| | | GTACTTCTGCATCTATCACACCTACCCTGATGGGACGTACACTGGGAGA |
| | | ATCTTCCTGGAGGTCCTAGAAAGCTCAGTGGCTGAGCACGGTGCCAGGT |
| | | TCCAGATTCCATTGCTTGGAGCCATGGCCGCGACGCTGGTGGTCATCTG |
| | | CACAGCAGTCATCGTGGTGGTCGCGTTGACTAGAAAGAAGAAAGCCCT |
| | | CAGAATCCATTCTGTGGAAGGTGACCTCAGGAGAAAATCAGCTGGACA |
| | | GGAGGAATGGAGCCCCAGTGCTCCCTCACCCCCAGGAAGCTGTGTCCA |
| | | GGCAGAAGCTGCACCTGCTGGGCTCTGTGGAGAGCAGCGGGGAGAGGA |
| | | CTGTGCCGAGCTGCATGACTACTTCAATGTCCTGAGTTACAGAAGCCTG |
| | | GGTAACTGCAGCTTCTTCACAGAGACTGGTTAGCAACCAGAGGCATCTT |
| | | CTGGAAGATACACTTTTGTCTTTGCTATTATAGATGAATATATAAGCAG |
| | | CTGTACTCTCCATCAGTGCTGCGTGTGTGTGTGTGTGTGTATGTGTGTGT |
| | | GTGTTCAGTTGAGTGAATAAATGTCATCCTCTTCTCCATCTTCATTTCCT |
| | | TGGCCTTTTCGTTCTATTCCATTTTGCATTATGGCAGGCCTAGGGTGAGT |
| | | AACGTGGATCTTGATCATAAATGCAAAATTAAAAAATATCTTGACCTGG |
| | | TTTTAAATCTGGCAGTTTGAGCAGATCCTATGTCTCTGAGAGACACATT |
| | | CCTCATAATGGCCAGCATTTTGGGCTACAAGGTTTTGTGGTTGATGATG |
| | | AGGATGGCATGACTGCAGAGCCATCCTCATCTCATTTTTTCACGTCATTT |
| | | TCAGTAACTTTCACTCATTCAAAGGCAGGTTATAAGTAAGTCCTGGTAG |
| | | CAGCCTCTATGGGGAGATTTGAGAGTGACTAAATCTTGGTATCTGCCCT |
| | | CAAGAACTTACAGTTAAATGGGGAGACAATGTTGTCATGAAAAGGTAT |
| | | TATAGTAAGGAGAGAAGGAGACATACACAGGCCTTCAGGAAGAGACGA |
| | | CAGTTTGGGGTGAGGTAGTTGGCATAGGCTTATCTGTGATGAAGTGGCC |
| | | TGGGAGCACCAAGGGGATGTTGAGGCTAGTCTGGGAGGAGCAGGAGTT |
| | | TTGTCTAGGGAACTTGTAGGAAATTCTTGGAGCTGAAAGTCCCACAAAG |
| | | AAGGCCCTGGCACCAAGGGAGTCAGCAAACTTCAGATTTTATTCTCTGG |
| | | GCAGGCATTTCAAGTTTCCTTTTGCTGTGACATACTCATCCATTAGACAG |
| | | CCTGATACAGGCCTGTAGCCTCTTCCGGCCGTGTGTGCTGGGGAAGCCC |
| | | CAGGAAACGCACATGCCCACACAGGGAGCCAAGTCGTAGCATTTGGGC |
| | | CTTGATCTACCTTTTCTGCATCAATACACTCTTGAGCCTTTGAAAAAAGA |
| | | ACGTTTCCCACTAAAAAGAAAATGTGGATTTTTAAAATAGGGACTCTTC |
| | | CTAGGGGAAAAAGGGGGGCTGGGAGTGATAGAGGGTTTAAAAAATAA |
| | | ACACCTTCAAACTAACTTCTTCGAACCCTTTTATTCACTCCCTGACGACT |
| | | TTGTGCTGGGGTTGGGGTAACTGAACCGCTTATTTCTGTTTAATTGCATT |
| | | CAGGCTGGATCTTAGAAGACTTTTATCCTTCCACCATCTCTCTCAGAGG |
| | | AATGAGCGGGGAGGTTGGATTTACTGGTGACTGATTTTCTTTCATGGGC |
| | | CAAGGAACTGAAAGAGAATGTGAAGCAAGGTTGTGTCTTGCGCATGGT |
| | | TAAAAATAAAGCATTGTCCTGCTTCCTAAGACTTAGACTGGGGTTGACA |
| | | ATTGTTTTAGCAACAAGACAATTCAACTATTTCTCCTAGGATTTTTATTA |
| | | TTATTATTTTTTCACTTTTCTACCAAATGGGTTACATAGGAAGAATGAAC |
| | | TGAAATCTGTCCAGAGCTCCAAGTCCTTTGGAAGAAAGATTAGATGAAC |
| | | GTAAAAATGTTGTTGTTTGCTGTGGCAGTTTACAGCATTTTTCTTGCAAA |
| | | ATTAGTGCAAATCTGTTGGAAATAGAACACAATTCACAAATTGGAAGTG |
| | | AACTAAAATGTAATGACGAAAAGGGAGTAGTGTTTTGATTTGGAGGAG |
| | | GTGTATATTCGGCAGAGGTTGGACTGAGAGTTGGGTGTTATTTAACATA |
| | | ATTATGGTAATTGGGAAACATTTATAAACACTATTGGGATGGTGATAAA |
| | | ATACAAAAGGGCCTATAGATGTTAGAAATGGGTCAGGTTACTGAAATG |
| | | GGATTCAATTTGAAAAAAATTTTTTTAAATAGAACTCACTGAACTAGAT |
| | | TCTCCTCTGAGAACCAGAGAAGACCATTTCATAGTTGGATTCCTGGAGA |
| | | CATGCGCTATCCACCACGTAGCCACTTTCCACATGTGGCCATCAACCAC |
| | | TTAAGATGGGGTTAGTTTAAATCAAGATGTGCTGTTATAATTGGTATAA |
| | | GCATAAAATCACACTAGATTCTGGAGATTTAA |
| | | TATGAATAATAAGAATACTATTTCAGTAGTTTTGGTATATTGTGTGTCAA |
| | | AAATGATAATATTTTGGATGTATTGGGTGAAATAAAATATTAACATTAA |
| | | AAAAAAAAA |
| Human TIGIT | 218 | MRWCLLLIWAQGLRQAPLASGMMTGTIETTGNISAEKGGSIILQCHLSSTT |
| protein (GenBank | | AQVTQVNWEQQDQLLAICNADLGWHISPSFKDRVAPGPGLGLTLQSLTVN |
| Accession No. | | DTGEYFCIYHTYPDGTYTGRIFLEVLESSVAEHGARFQIPLLGAMAATLVVI |
| NP_776160.2) | | CTAVIVVVALTRKKKALRIHSVEGDLRRKSAGQEEWSPSAPSPPGSCVQAE |
| | | AAPAGLCGEQRGEDCAELHDYFNVLSYRSLGNCSFFTETG |
| Cynomolgus | 219 | MAFLVAPPMQFVYLLKTLCVFNMVFAKPGFSETVFSHRLSFTVLSAVGYFR |
| monkey TIGIT | | WQKRPHLLPVSPLGRSMRWCLFLIWAQGLRQAPLASGMMTGTIETTGNIS |
| protein | | AKKGGSVILQCHLSSTMAQVTQVNWEQHDHSLLAIRNAELGWHIYPAFKD |
| | | RVAPGPGLGLTLQSLTMNDTGEYFCTYHTYPDGTYRGRIFLEVLESSVAEH |
| | | SARFQIPLLGAMAMMLVVICIAVIVVVVLARKKKSLRIHSVESGLQRKSTG |
| | | QEEQIPSAPSPPGSCVQAEAAPAGLCGEQQGDDCAELHDYFNVLSYRSLGS |
| | | CSFFTETG |
| Mouse TIGIT | 220 | MHGWLLLVWVQGLIQAAFLATGATAGTIDTKRNISAEEGGSVILQCHFSSD |
| protein | | TAEVTQVDWKQQDQLLAIYSVDLGWHVASVFSDRVVPGPSLGLTFQSLTM |
| | | NDTGEYFCTYHTYPGGIYKGRIFLKVQESSVAQFQTAPLGGTMAAVLGLIC |
| | | LMVTGVTVLARKKSIRMHSIESGLGRTEAEPQEWNLRSLSSPGSPVQTQTA |
| | | PAGPCGEQAEDDYADPQEYFNVLSYRSLESFIAVSKTG |
| Clone 2C VH CDR1 | 221 | FTFTDYYMD |
| Clone 2C VH CDR2 | 222 | RTRNKVNSYYTEYAASVKG |
| Clone 2C VH CDR3 | 223 | ARGQYYYGSDRRGYYYMDV |
| Clones 13A, 13C, | 224 | GTFLSSAIS |
| and 13D VH CDR1 | | |
| Clone 13A VH | 225 | SLIPYFGTANYAQKFQG |
| CDR2 | | |
| Clone 13B VH | 226 | GTFSAWAIS |
| CDR1 | | |
| Clones 13B and 13D | 227 | SIIPYFGKANYAQKFQG |
| VH CDR2 | | |
| Clone 13B VH | 228 | ARGPSEVSGILGYVWFDP |
| CDR3 | | |
| Clone 13C VH | 229 | SIIPLFGKANYAQKFQG |
| CDR2 | | |
| Clones 13C and 13D | 230 | ARGPSEVKGILGYVWFDP |
| VH CDR3 | | |
| Clone 16C VH | 231 | GTFREYAIS |
| CDR1 | | |
| Clone 16C VH | 232 | GIHPIFGTARYAQKFQG |
| CDR2 | | |
| Clones 16D and 16E | 233 | GTFSDYPIS |
| VH CDR1 | | |
| Clones 16D, and | 234 | GIIPIVGGANYAQKFQG |
| 16E VH CDR2 | | |
| Clone 16C VH | 235 | TRQSTWHKLYGTDV |
| CDR3 | | |
| Clone 16D VH | 236 | TRQSTWHKLFGTDV |
| CDR3 | | |
| Clone 16E VH | 237 | ARQSTWHKVYGTDV |
| CDR3 | | |
| Clone 25A VH | 238 | WISAYNGNTKYAQKLQG |
| CDR2 | | |
| Clones 25B, 25C, | 239 | YTFTSYPIG |
| and 25D VH CDR1 | | |
| Clones 25B, 25C, | 240 | WISSYNGNTNYAQKLQG |
| and 25D VH CDR2 | | |
| Clone 25C VH | 241 | ARGASSFWSGDVLGAFDI |
| CDR3 | | |
| Clone 25D VH | 242 | ARDLKSFWSGDVLGAFDI |
| CDR3 | | |
| Clone 25E VH | 243 | YTFTSYAIA |
| CDR1 | | |
| Clone 25E VH | 244 | ARSGSSFWSGDVLGAFDI |
| CDR3 | | |
| Clone 2C VH | 245 | EVQLVESGGGLVQPGGSLRLSCAASGFTFTDYYMDWVRQAPGKGLEWVG |
| | | RTRNKVNSYYTEYAASVKGRFTISRDDSKNSLYLQMNSLKTEDTAVYYCA |
| | | RGQYYYGSDRRGYYYMDVWGQGTTVTVSS |
| Clone 13A VH | 246 | QVQLVQSGAEVKKPGSSVKVSCKASGGTFLSSAISWVRQAPGQGLEWMGS |
| | | LIPYFGTANYAQKFQGRVTITADESTSTAYMELSSLRSEDTAVYYCARGPSE |
| | | VGAILGYVWFDPWGQGTLVTVSS |
| Clone 13B VH | 247 | QVQLVQSGAEVKKPGSSVKVSCKASGGTFSAWAISWVRQAPGQGLEWMG |
| | | SIIPYFGKANYAQKFQGRVTITADESTSTAYMELSSLRSEDTAVYYCARGPS |
| | | EVSGILGYVWFDPWGQGTLVTVSS |
| Clone 13C VH | 248 | QVQLVQSGAEVKKPGSSVKVSCKASGGTFLSSAISWVRQAPGQGLEWMGS |
| | | IIPLFGKANYAQKFQGRVTITADESTSTAYMELSSLRSEDTAVYYCARGPSE |
| | | VKGILGYVWFDPWGQGTLVTVSS |
| Clone 13D VH | 249 | QVQLVQSGAEVKKPGSSVKVSCKASGGTFLSSAISWVRQAPGQGLEWMGS |
| | | IIPYFGKANYAQKFQGRVTITADESTSTAYMELSSLRSEDTAVYYCARGPSE |
| | | VKGILGYVWFDPWGQGTLVTVSS |
| Clone 16C VH | 250 | QVQLVQSGAEVKKPGSSVKVSCKASGGTFREYAISWVRQAPGQGLEWMG |
| | | GIHPIFGTARYAQKFQGRVTITADESTSTAYMELSSLRSEDTAVYYCTRQST |
| | | WHKLYGTDVWGQGTTVTVSS |
| Clone 16D VH | 251 | QVQLVQSGAEVKKPGSSVKVSCKASGGTFSDYPISWVRQAPGQGLEWMG |
| | | GIIPIVGGANYAQKFQGRVTITADESTSTAYMELSSLRSEDTAVYYCTRQST |
| | | WHKLFGTDVWGQGTTVTVSS |
| Clone 16E VH | 252 | QVQLVQSGAEVKKPGSSVKVSCKASGGTFSDYPISWVRQAPGQGLEWMG |
| | | GIIPIVGGANYAQKFQGRVTITADESTSTAYMELSSLRSEDTAVYYCARQST |
| | | WHKVYGTDVWGQGTTVTVSS |
| Clone 25A VH | 253 | QVQLVQSGAEVKKPGASVKVSCKASGYTFTSYAISWVRQAPGQGLEWMG |
| | | WISAYNGNTKYAQKLQGRVTMTTDTSTSTAYMELRSLRSDDTAVYYCAR |
| | | DLSSFWSGDVLGAFDIWGQGTMVTVSS |
| Clone 25B VH | 254 | QVQLVQSGAEVKKPGASVKVSCKASGYTFTSYPIGWVRQAPGQGLEWMG |
| | | WISSYNGNTNYAQKLQGRVTMTTDTSTSTAYMELRSLRSDDTAVYYCAR |
| | | DLSSFWSGDVLGAFDIWGQGTMVTVSS |
| Clone 25C VH | 255 | QVQLVQSGAEVKKPGASVKVSCKASGYTFTSYPIGWVRQAPGQGLEWMG |
| | | WISSYNGNTNYAQKLQGRVTMTTDTSTSTAYMELRSLRSDDTAVYYCAR |
| | | GASSFWSGDVLGAFDIWGQGTMVTVSS |
| Clone 25D VH | 256 | QVQLVQSGAEVKKPGASVKVSCKASGYTFTSYPIGWVRQAPGQGLEWMG |
| | | WISSYNGNTNYAQKLQGRVTMTTDTSTSTAYMELRSLRSDDTAVYYCAR |
| | | DLKSFWSGDVLGAFDIWGQGTMVTVSS |
| Clone 25E VH | 257 | QVQLVQSGAEVKKPGASVKVSCKASGYTFTSYAIAWVRQAPGQGLEWMG |
| | | WISAYNGNTNYAQKLQGRVTMTTDTSTSTAYMELRSLRSDDTAVYYCAR |
| | | SGSSFWSGDVLGAFDIWGQGTMVTVSS |
| hTIGIT 68-82 | 258 | ICNADLGWHISPSFK |
| epitope | | |

Although the foregoing invention has been described in some detail by way of illustration and example for purposes of clarity of understanding, one of skill in the art will appreciate that many modifications and variations of this invention can be made without departing from its spirit and scope. The specific embodiments described herein are offered by way of example only and are not meant to be limiting in any way. It is intended that the specification and examples be considered as exemplary only, with the true scope and spirit of the invention being indicated by the following claims.

All publications, patents, patent applications or other documents cited herein are hereby incorporated by reference in their entirety for all purposes to the same extent as if each individual publication, patent, patent application, or other document was individually indicated to be incorporated by reference for all purposes.

1.-69. (canceled)

70. An isolated antibody or antigen-binding portion thereof that binds to human TIGIT (T-cell immunoreceptor with Ig and ITIM domains), wherein the antibody or antigen-binding portion thereof comprises a heavy chain CDR1, CDR2, and CDR3 and a light chain CDR1, CDR2, and CDR3 comprising the sequences of:
(a) SEQ ID NOs: 4, 6, 8, 13, 15, and 17, respectively; or
(b) SEQ ID NOs: 22, 24, 26, 31, 33, and 35, respectively; or
(c) SEQ ID NOs: 40, 42, 44, 49, 51, and 53, respectively; or
(d) SEQ ID NOs: 76, 78, 80, 85, 87, and 89, respectively; or
(e) SEQ ID NOs: 94, 96, 98, 103, 105, and 107, respectively; or
(f) SEQ ID NOs: 112, 114, 116, 121, 123, and 125, respectively; or
(g) SEQ ID NOs: 130, 132, 134, 139, 141, and 143, respectively; or
(h) SEQ ID NOs: 148, 150, 152, 157, 159, and 161, respectively; or
(i) SEQ ID NOs: 166, 168, 170, 175, 177, and 179, respectively; or
(j) SEQ ID NOs: 184, 186, 188, 193, 195, and 197, respectively; or
(k) SEQ ID NOs: 202, 204, 206, 211, 213, and 215, respectively; or
(l) SEQ ID NOs: 221, 222, 223, 13, 15, and 17, respectively; or
(m) SEQ ID NOs: 231, 232, 235, 103, 105, and 107, respectively; or
(n) SEQ ID NOs: 233, 234, 236, 103, 105, and 107, respectively; or
(o) SEQ ID NOs: 233, 234, 237, 103, 105, and 107, respectively; or
(p) SEQ ID NOs: 166, 238, 170, 175, 177, and 179, respectively; or
(q) SEQ ID NOs: 239, 240, 170, 175, 177, and 179, respectively; or
(r) SEQ ID NOs: 239, 240, 241, 175, 177, and 179, respectively; or
(s) SEQ ID NOs: 239, 240, 242, 175, 177, and 179, respectively; or
(t) SEQ ID NOs: 243, 168, 244, 175, 177, and 179, respectively.

71. The isolated antibody or antigen-binding portion thereof of claim 70, wherein the antibody or antigen-binding portion thereof comprises:
(a) a heavy chain variable region comprising the amino acid sequence of SEQ ID NO:1 or SEQ ID NO:245 and a light chain variable region comprising the amino acid sequence of SEQ ID NO: 10; or
(b) a heavy chain variable region comprising the amino acid sequence of SEQ ID NO:19 and a light chain variable region comprising the amino acid sequence of SEQ ID NO:28; or
(c) a heavy chain variable region comprising the amino acid sequence of SEQ ID NO:37 and a light chain variable region comprising the amino acid sequence of SEQ ID NO:46; or
(d) a heavy chain variable region comprising the amino acid sequence of SEQ ID NO:73 and a light chain variable region comprising the amino acid sequence of SEQ ID NO:82; or
(e) a heavy chain variable region comprising the amino acid sequence of SEQ ID NO:91, SEQ ID NO:250, SEQ ID NO:251, or SEQ ID NO:252 and a light chain variable region comprising the amino acid sequence of SEQ ID NO:100; or
(f) a heavy chain variable region comprising the amino acid sequence of SEQ ID NO:109 and a light chain variable region comprising the amino acid sequence of SEQ ID NO:118; or
(g) a heavy chain variable region comprising the amino acid sequence of SEQ ID NO:127 and a light chain variable region comprising the amino acid sequence of SEQ ID NO:136; or
(h) a heavy chain variable region comprising the amino acid sequence of SEQ ID NO:145 and a light chain variable region comprising the amino acid sequence of SEQ ID NO:154; or
(i) a heavy chain variable region comprising the amino acid sequence of SEQ ID NO:163, SEQ ID NO:253, SEQ ID NO:254, SEQ ID NO:255, SEQ ID NO:256, or SEQ ID NO:257 and a light chain variable region comprising the amino acid sequence of SEQ ID NO:172; or
(j) a heavy chain variable region comprising the amino acid sequence of SEQ ID NO:181 and a light chain variable region comprising the amino acid sequence of SEQ ID NO:190; or
(k) a heavy chain variable region comprising the amino acid sequence of SEQ ID NO:199 and a light chain variable region comprising the amino acid sequence of SEQ ID NO:208.

72. A pharmaceutical composition comprising the isolated antibody or antigen-binding portion thereof of claim 70 and a pharmaceutically acceptable carrier.

73. A bispecific antibody comprising the antibody or antigen-binding portion thereof of claim 70.

74. An antibody-drug conjugate comprising the antibody or antigen-binding portion thereof of claim 70 conjugated to a cytotoxic agent.

75. An isolated polynucleotide that encodes an antibody or antigen-binding portion thereof that binds to human TIGIT (T-cell immunoreceptor with Ig and ITIM domains), wherein the antibody or antigen-binding portion thereof comprises a heavy chain CDR1, CDR2, and CDR3 and a light chain CDR1, CDR, and CDR3 comprising the sequences of:
(a) SEQ ID NOs: 4, 6, 8, 13, 15, and 17, respectively; or
(b) SEQ ID NOs: 22, 24, 26, 31, 33, and 35, respectively; or
(c) SEQ ID NOs: 40, 42, 44, 49, 51, and 53, respectively; or
(d) SEQ ID NOs: 76, 78, 80, 85, 87, and 89, respectively; or
(e) SEQ ID NOs: 94, 96, 98, 103, 105, and 107, respectively; or
(f) SEQ ID NOs: 112, 114, 116, 121, 123, and 125, respectively; or
(g) SEQ ID NOs: 130, 132, 134, 139, 141, and 143, respectively; or
(h) SEQ ID NOs: 148, 150, 152, 157, 159, and 161, respectively; or
(i) SEQ ID NOs: 166, 168, 170, 175, 177, and 179, respectively; or
(j) SEQ ID NOs: 184, 186, 188, 193, 195, and 197, respectively; or
(k) SEQ ID NOs: 202, 204, 206, 211, 213, and 215, respectively; or
(l) SEQ ID NOs: 221, 222, 223, 13, 15, and 17, respectively; or
(m) SEQ ID NOs: 231, 232, 235, 103, 105, and 107, respectively; or
(n) SEQ ID NOs: 233, 234, 236, 103, 105, and 107, respectively; or
(o) SEQ ID NOs: 233, 234, 237, 103, 105, and 107, respectively; or
(p) SEQ ID NOs: 166, 238, 170, 175, 177, and 179, respectively; or
(q) SEQ ID NOs: 239, 240, 170, 175, 177, and 179, respectively; or
(r) SEQ ID NOs: 239, 240, 241, 175, 177, and 179, respectively; or
(s) SEQ ID NOs: 239, 240, 242, 175, 177, and 179, respectively; or
(t) SEQ ID NOs: 243, 168, 244, 175, 177, and 179, respectively.

76. The isolated polynucleotide of claim 75, wherein the isolated polynucleotide comprises:
(a) the nucleotide sequence of SEQ ID NO: 2 and/or the nucleotide sequence of SEQ ID NO: 11; or
(b) the nucleotide sequence of SEQ ID NO: 20 and/or the nucleotide sequence of SEQ ID NO: 29; or
(c) the nucleotide sequence of SEQ ID NO: 38 and/or the nucleotide sequence of SEQ ID NO: 47; or
(d) the nucleotide sequence of SEQ ID NO: 74 and/or the nucleotide sequence of SEQ ID NO: 83; or
(e) the nucleotide sequence of SEQ ID NO: 92 and/or the nucleotide sequence of SEQ ID NO: 101; or
(f) the nucleotide sequence of SEQ ID NO: 110 and/or the nucleotide sequence of SEQ ID NO: 119; or
(g) the nucleotide sequence of SEQ ID NO: 128 and/or the nucleotide sequence of SEQ ID NO: 137; or
(h) the nucleotide sequence of SEQ ID NO: 146 and/or the nucleotide sequence of SEQ ID NO: 155; or
(i) the nucleotide sequence of SEQ ID NO: 164 and/or the nucleotide sequence of SEQ ID NO: 173; or
(j) the nucleotide sequence of SEQ ID NO: 182 and/or the nucleotide sequence of SEQ ID NO: 191; or
(k) the nucleotide sequence of SEQ ID NO: 200 and/or the nucleotide sequence of SEQ ID NO: 209.

77. A vector comprising the isolated polynucleotide of claim 75.

78. A host cell comprising the isolated polynucleotide of claim 75.

79. A host cell that expresses the antibody or antigen-binding portion thereof of claim 70.

80. A method of producing an antibody or antigen-binding portion thereof, comprising culturing the host cell of claim 79 under compositions suitable for producing the antibody or antigen-binding portion thereof.

81. A kit comprising the antibody or antigen-binding portion thereof of claim 70 and an immuno-oncology agent.

82. A method of treating cancer in a subject, comprising administering to the subject a therapeutic amount of an antibody or antigen-binding portion thereof that binds to human TIGIT (T-cell immunoreceptor with Ig and ITIM domains), wherein the antibody or antigen-binding portion thereof comprises a heavy chain CDR1, CDR2, and CDR3 and a light chain CDR1, CDR, and CDR3 comprising the sequences of:
(a) SEQ ID NOs: 4, 6, 8, 13, 15, and 17, respectively; or
(b) SEQ ID NOs: 22, 24, 26, 31, 33, and 35, respectively; or
(c) SEQ ID NOs: 40, 42, 44, 49, 51, and 53, respectively; or
(d) SEQ ID NOs: 76, 78, 80, 85, 87, and 89, respectively; or
(e) SEQ ID NOs: 94, 96, 98, 103, 105, and 107, respectively; or
(f) SEQ ID NOs: 112, 114, 116, 121, 123, and 125, respectively; or
(g) SEQ ID NOs: 130, 132, 134, 139, 141, and 143, respectively; or
(h) SEQ ID NOs: 148, 150, 152, 157, 159, and 161, respectively; or
(i) SEQ ID NOs: 166, 168, 170, 175, 177, and 179, respectively; or
(j) SEQ ID NOs: 184, 186, 188, 193, 195, and 197, respectively; or
(k) SEQ ID NOs: 202, 204, 206, 211, 213, and 215, respectively; or
(l) SEQ ID NOs: 221, 222, 223, 13, 15, and 17, respectively; or
(m) SEQ ID NOs: 231, 232, 235, 103, 105, and 107, respectively; or
(n) SEQ ID NOs: 233, 234, 236, 103, 105, and 107, respectively; or
(o) SEQ ID NOs: 233, 234, 237, 103, 105, and 107, respectively; or
(p) SEQ ID NOs: 166, 238, 170, 175, 177, and 179, respectively; or
(q) SEQ ID NOs: 239, 240, 170, 175, 177, and 179, respectively; or
(r) SEQ ID NOs: 239, 240, 241, 175, 177, and 179, respectively; or
(s) SEQ ID NOs: 239, 240, 242, 175, 177, and 179, respectively; or
(t) SEQ ID NOs: 243, 168, 244, 175, 177, and 179, respectively.

83. The method of claim 82, wherein the cancer is bladder cancer, breast cancer, uterine cancer, cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, or sarcoma.

84. The method of claim 82, wherein the method further comprises administering to the subject a therapeutic amount of an immuno-oncology agent.


SEQUENCE LISTING (270 sequences)

SEQ ID NO: 1 - protein, 128 aa, Artificial Sequence
  Synthetic construct
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCARGQYYYGSSSRGYYYMDVWGQGTTVTVSS

SEQ ID NO: 2 - DNA, 384 bp, Artificial Sequence
  Synthetic construct
gaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctaacagttacaccacagaatacgccgcgtctgtgaaaggcagattcaccatctcaagagatgattcaaagaactcactgtatctgcaaatgaacagcctgaaaaccgaggacacggcggtgtactactgcgccagaggccagtactactacggcagcagcagcagaggttactactacatggacgtatggggccagggaacaaccgtcaccgtctcctca

SEQ ID NO: 3 - protein, 26 aa, Artificial Sequence
  Synthetic construct
EVQLVESGGGLVQPGGSLRLSCAASG

SEQ ID NO: 4 - protein, 9 aa, Artificial Sequence
  Synthetic construct
FTFSDHYMD

SEQ ID NO: 5 - protein, 14 aa, Artificial Sequence
  Synthetic construct
WVRQAPGKGLEWVG

SEQ ID NO: 6 - protein, 19 aa, Artificial Sequence
  Synthetic construct
RTRNKANSYTTEYAASVKG

SEQ ID NO: 7 - protein, 30 aa, Artificial Sequence
  Synthetic construct
RFTISRDDSKNSLYLQMNSLKTEDTAVYYC

SEQ ID NO: 8 - protein, 19 aa, Artificial Sequence
  Synthetic construct
ARGQYYYGSSSRGYYYMDV

SEQ ID NO: 9 - protein, 11 aa, Artificial Sequence
  Synthetic construct
WGQGTTVTVSS

SEQ ID NO: 10 - protein, 108 aa, Artificial Sequence
  Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAVPSPLTFGGGTKVEIK

SEQ ID NO: 11 - DNA, 324 bp, Artificial Sequence
  Synthetic construct
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaggccgtccccagtcctctcacttttggcggagggaccaaggttgagatcaaa

SEQ ID NO: 12 - protein, 23 aa, Artificial Sequence
  Synthetic construct
EIVLTQSPGTLSLSPGERATLSC

SEQ ID NO: 13 - protein, 12 aa, Artificial Sequence
  Synthetic construct
RASQSVSSSYLA

SEQ ID NO: 14 - protein, 15 aa, Artificial Sequence
  Synthetic construct
WYQQKPGQAPRLLIY

SEQ ID NO: 15 - protein, 7 aa, Artificial Sequence
  Synthetic construct
GASSRAT

SEQ ID NO: 16 - protein, 32 aa, Artificial Sequence
  Synthetic construct
GIPDRFSGSGSGTDFTLTISRLEPEDFAVYYC

SEQ ID NO: 17 - protein, 9 aa, Artificial Sequence
  Synthetic construct
QQAVPSPLT

SEQ ID NO: 18 - protein, 10 aa, Artificial Sequence
  Synthetic construct
FGGGTKVEIK

SEQ ID NO: 19 - protein, 128 aa, Artificial Sequence; identical to SEQ ID NO: 1
  Synthetic construct

SEQ ID NO: 20 - DNA, 384 bp, Artificial Sequence; identical to SEQ ID NO: 2
  Synthetic construct

SEQ ID NO: 21 - protein, 26 aa, Artificial Sequence
  Synthetic construct
EVQLVESGGGLVQPGGSLRLSCAASG

SEQ ID NO: 22 - protein, 9 aa, Artificial Sequence
  Synthetic construct
FTFSDHYMD

SEQ ID NO: 23 - protein, 14 aa, Artificial Sequence
  Synthetic construct
WVRQAPGKGLEWVG

SEQ ID NO: 24 - protein, 19 aa, Artificial Sequence
  Synthetic construct
RTRNKANSYTTEYAASVKG

SEQ ID NO: 25 - protein, 30 aa, Artificial Sequence; identical to SEQ ID NO: 7
  Synthetic construct

SEQ ID NO: 26 - protein, 19 aa, Artificial Sequence
  Synthetic construct
ARGQYYYGSSSRGYYYMDV

SEQ ID NO: 27 - protein, 11 aa, Artificial Sequence
  Synthetic construct
WGQGTTVTVSS

SEQ ID NO: 28 - protein, 107 aa, Artificial Sequence
  Synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQVGPPLTFGGGTKVEIK

SEQ ID NO: 29 - DNA, 321 bp, Artificial Sequence
  Synthetic construct
gaaattgtgttgacgcagtctccaggcaccctgtctttgtctccaggggaaagagccaccctctcctgcagggccagtcagagtgttaggagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaggtcggaccccccctcacttttggcggagggaccaaggttgagatcaaa

SEQ ID NO: 30 - protein, 23 aa, Artificial Sequence
  Synthetic construct
EIVLTQSPGTLSLSPGERATLSC

SEQ ID NO: 31 - protein, 12 aa, Artificial Sequence
  Synthetic construct
RASQSVRSSYLA

SEQ ID NO: 32 - protein, 15 aa, Artificial Sequence
  Synthetic construct
WYQQKPGQAPRLLIY

SEQ ID NO: 33 - protein, 7 aa, Artificial Sequence
  Synthetic construct
GASSRAT

SEQ ID NO: 34 - protein, 32 aa, Artificial Sequence; identical to SEQ ID NO: 16
  Synthetic construct

SEQ ID NO: 35 - protein, 8 aa, Artificial Sequence
  Synthetic construct
QQVGPPLT

SEQ ID NO: 36 - protein, 10 aa, Artificial Sequence
  Synthetic construct
FGGGTKVEIK

SEQ ID NO: 37 - protein, 121 aa, Artificial Sequence
  Synthetic construct
EVQLLESGGGLVQPGGSLRLSCAASGFTFSTYAMSWVRQAPGKGLEWVSAISGSGGSTYYADSVKGRFTISRDNSKNTLYLQMNSLRAEDTAVYYCAKGPRYQDRAGMDVWGQGTTVTVSS

SEQ ID NO: 38 - DNA, 363 bp, Artificial Sequence
  Synthetic construct
gaggtgcagctgttggagtctgggggaggcttggtacagcctggggggtccctgagactctcctgtgcagcctctggattcacctttagcacctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggcggtgtactactgcgccaagggccccagataccaagacagggcaggaatggacgtatggggccagggaacaactgtcaccgtctcctca

SEQ ID NO: 39 - protein, 26 aa, Artificial Sequence
  Synthetic construct
EVQLLESGGGLVQPGGSLRLSCAASG

SEQ ID NO: 40 - protein, 9 aa, Artificial Sequence
  Synthetic construct
FTFSTYAMS

SEQ ID NO: 41 - protein, 14 aa, Artificial Sequence
  Synthetic construct
WVRQAPGKGLEWVS

SEQ ID NO: 42 - protein, 17 aa, Artificial Sequence
  Synthetic construct
AISGSGGSTYYADSVKG

SEQ ID NO: 43 - protein, 30 aa, Artificial Sequence
  Synthetic construct
RFTISRDNSKNTLYLQMNSLRAEDTAVYYC

SEQ ID NO: 44 - protein, 14 aa, Artificial Sequence
  Synthetic construct
AKGPRYQDRAGMDV

SEQ ID NO: 45 - protein, 11 aa, Artificial Sequence
  Synthetic construct
WGQGTTVTVSS

SEQ ID NO: 46 - protein, 107 aa, Artificial Sequence
  Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSLATPYTFGGGTKVEIK

SEQ ID NO: 47 - DNA, 321 bp, Artificial Sequence
  Synthetic construct
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagcctcgccactccttacacttttggcggagggaccaaggttgagatcaaa

SEQ ID NO: 48 - protein, 23 aa, Artificial Sequence
  Synthetic construct
DIQMTQSPSSLSASVGDRVTITC

SEQ ID NO: 49 - protein, 11 aa, Artificial Sequence
  Synthetic construct
RASQSISSYLN

SEQ ID NO: 50 - protein, 15 aa, Artificial Sequence
  Synthetic construct
WYQQKPGKAPKLLIY

SEQ ID NO: 51 - protein, 7 aa, Artificial Sequence
  Synthetic construct
AASSLQS

SEQ ID NO: 52 - protein, 32 aa, Artificial Sequence
  Synthetic construct
GVPSRFSGSGSGTDFTLTISSLQPEDFATYYC

SEQ ID NO: 53 - protein, 9 aa, Artificial Sequence
  Synthetic construct
QQSLATPYT

SEQ ID NO: 54 - protein, 10 aa, Artificial Sequence
  Synthetic construct
FGGGTKVEIK

SEQ ID NO: 55 - protein, 125 aa, Artificial Sequence
  Synthetic construct
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSS

SEQ ID NO: 56 - DNA, 375 bp, Artificial Sequence
  Synthetic construct
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaagcatcatccctatctttggtacagcaaactacgcacagaagttccagggcagagtcacgattaccgcggacgaatccacgagcacagcctacatggagctgagcagcctgagatctgaggacacggcggtgtactactgcgccagaggcccttctgaagtaggagcaatactcggatatgtatggttcgacccatggggacagggtacattggtcaccgtctcctca

SEQ ID NO: 57 - protein, 26 aa, Artificial Sequence
  Synthetic construct
QVQLVQSGAEVKKPGSSVKVSCKASG

SEQ ID NO: 58 - protein, 9 aa, Artificial Sequence
  Synthetic construct
GTFSSYAIS

SEQ ID NO: 59 - protein, 14 aa, Artificial Sequence
  Synthetic construct
WVRQAPGQGLEWMG

SEQ ID NO: 60 - protein, 17 aa, Artificial Sequence
  Synthetic construct
SIIPIFGTANYAQKFQG

SEQ ID NO: 61 - protein, 30 aa, Artificial Sequence
  Synthetic construct
RVTITADESTSTAYMELSSLRSEDTAVYYC

SEQ ID NO: 62 - protein, 18 aa, Artificial Sequence
  Synthetic construct
ARGPSEVGAILGYVWFDP

SEQ ID NO: 63 - protein, 11 aa, Artificial Sequence
  Synthetic construct
WGQGTLVTVSS

SEQ ID NO: 64 - protein, 112 aa, Artificial Sequence
  Synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIK

SEQ ID NO: 65 - DNA, 336 bp, Artificial Sequence
  Synthetic construct
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaggcaagacgaatccctatcacttttggcggagggaccaaggttgagatcaaa

SEQ ID NO: 66 - protein, 23 aa, Artificial Sequence
  Synthetic construct
DIVMTQSPLSLPVTPGEPASISC

SEQ ID NO: 67 - protein, 16 aa, Artificial Sequence
  Synthetic construct
RSSQSLLHSNGYNYLD

SEQ ID NO: 68 - protein, 15 aa, Artificial Sequence
  Synthetic construct
WYLQKPGQSPQLLIY

SEQ ID NO: 69 - protein, 7 aa, Artificial Sequence
  Synthetic construct
LGSNRAS

SEQ ID NO: 70 - protein, 32 aa, Artificial Sequence
  Synthetic construct
GVPDRFSGSGSGTDFTLKISRVEAEDVGVYYC

SEQ ID NO: 71 - protein, 9 aa, Artificial Sequence
  Synthetic construct
MQARRIPIT

SEQ ID NO: 72 - protein, 10 aa, Artificial Sequence
  Synthetic construct
FGGGTKVEIK

SEQ ID NO: 73 - protein, 125 aa, Artificial Sequence; identical to SEQ ID NO: 55
  Synthetic construct

SEQ ID NO: 74 - DNA, 375 bp, Artificial Sequence; identical to SEQ ID NO: 56
  Synthetic construct

SEQ ID NO: 75 - protein, 26 aa, Artificial Sequence
  Synthetic construct
QVQLVQSGAEVKKPGSSVKVSCKASG

SEQ ID NO: 76 - protein, 9 aa, Artificial Sequence
  Synthetic construct
GTFSSYAIS

SEQ ID NO: 77 - protein, 14 aa, Artificial Sequence
  Synthetic construct
WVRQAPGQGLEWMG

SEQ ID NO: 78 - protein, 17 aa, Artificial Sequence
  Synthetic construct
SIIPIFGTANYAQKFQG

SEQ ID NO: 79 - protein, 30 aa, Artificial Sequence; identical to SEQ ID NO: 61
  Synthetic construct

SEQ ID NO: 80 - protein, 18 aa, Artificial Sequence
  Synthetic construct
ARGPSEVGAILGYVWFDP

SEQ ID NO: 81 - protein, 11 aa, Artificial Sequence
  Synthetic construct
WGQGTLVTVSS

SEQ ID NO: 82 - protein, 112 aa, Artificial Sequence
  Synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQAKRLPLTFGGGTKVEIK

SEQ ID NO: 83 - DNA, 336 bp, Artificial Sequence
  Synthetic construct
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaggcaaaacgactccctctcacttttggcggagggaccaaggttgagatcaaa

SEQ ID NO: 84 - protein, 23 aa, Artificial Sequence
  Synthetic construct
DIVMTQSPLSLPVTPGEPASISC

SEQ ID NO: 85 - protein, 16 aa, Artificial Sequence
  Synthetic construct
RSSQSLLHSNGYNYLD

SEQ ID NO: 86 - protein, 15 aa, Artificial Sequence
  Synthetic construct
WYLQKPGQSPQLLIY

SEQ ID NO: 87 - protein, 7 aa, Artificial Sequence
  Synthetic construct
LGSNRAS

SEQ ID NO: 88 - protein, 32 aa, Artificial Sequence; identical to SEQ ID NO: 70
  Synthetic construct

SEQ ID NO: 89 - protein, 9 aa, Artificial Sequence
  Synthetic construct
MQAKRLPLT

SEQ ID NO: 90 - protein, 10 aa, Artificial Sequence
  Synthetic construct
FGGGTKVEIK

SEQ ID NO: 91 - protein, 121 aa, Artificial Sequence
  Synthetic construct
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQSTWHKLYGTDVWGQGTTVTVSS

SEQ ID NO: 92 - DNA, 363 bp, Artificial Sequence
  Synthetic construct
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaagctacgcacagaagttccagggcagagtcacgattaccgcggacgaatccacgagcacagcctacatggagctgagcagcctgagatctgaggacacggcggtgtactactgcgcaagacagagcacctggcacaaattgtacggaacggacgtatggggccagggaacaactgtcaccgtctcctca

SEQ ID NO: 93 - protein, 26 aa, Artificial Sequence
  Synthetic construct
QVQLVQSGAEVKKPGSSVKVSCKASG

SEQ ID NO: 94 - protein, 9 aa, Artificial Sequence
  Synthetic construct
GTFSSYAIS

SEQ ID NO: 95 - protein, 14 aa, Artificial Sequence
  Synthetic construct
WVRQAPGQGLEWMG

SEQ ID NO: 96 - protein, 17 aa, Artificial Sequence
  Synthetic construct
GIIPIFGTASYAQKFQG

SEQ ID NO: 97 - protein, 30 aa, Artificial Sequence; identical to SEQ ID NO: 61
  Synthetic construct

SEQ ID NO: 98 - protein, 14 aa, Artificial Sequence
  Synthetic construct
ARQSTWHKLYGTDV

SEQ ID NO: 99 - protein, 11 aa, Artificial Sequence
  Synthetic construct
WGQGTTVTVSS

SEQ ID NO: 100 - protein, 107 aa, Artificial Sequence
  Synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGDSLPPTFGGGTKVEIK

SEQ ID NO: 101 - DNA, 321 bp, Artificial Sequence
  Synthetic construct
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcagggagacagtctccctcctacttttggcggagggaccaaggttgagatcaaa

SEQ ID NO: 102 - protein, 23 aa, Artificial Sequence
  Synthetic construct
DIQMTQSPSSVSASVGDRVTITC

SEQ ID NO: 103 - protein, 11 aa, Artificial Sequence
  Synthetic construct
RASQGISSWLA

SEQ ID NO: 104 - protein, 15 aa, Artificial Sequence
  Synthetic construct
WYQQKPGKAPKLLIY

SEQ ID NO: 105 - protein, 7 aa, Artificial Sequence
  Synthetic construct
AASSLQS

SEQ ID NO: 106 - protein, 32 aa, Artificial Sequence; identical to SEQ ID NO: 52
  Synthetic construct

SEQ ID NO: 107 - protein, 9 aa, Artificial Sequence
  Synthetic construct
QQGDSLPPT

SEQ ID NO: 108 - protein, 10 aa, Artificial Sequence
  Synthetic construct
FGGGTKVEIK

SEQ ID NO: 109 - protein, 120 aa, Artificial Sequence
  Synthetic construct
QVQLVQSGAEVKKPGASVKVSCKASGYTFTSYYMSWVRQAPGQGLEWMGIINPSGGSTSYAQKFQGRVTMTRDTSTSTVYMELSSLRSEDTAVYYCARVRYGYADGMDVWGQGTTVTVSS

SEQ ID NO: 110 - DNA, 360 bp, Artificial Sequence
  Synthetic construct
caggtgcagctggtgcagtctggggctgaggtgaagaagcctggggcctcagtgaaggtttcctgcaaggcatctggatacaccttcaccagctactatatgtcatgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggcggtgtactactgcgccagagtgaggtacggatacgcagacggaatggacgtatggggccagggaacaactgtcaccgtctcctca

SEQ ID NO: 111 - protein, 26 aa, Artificial Sequence
  Synthetic construct
QVQLVQSGAEVKKPGASVKVSCKASG

SEQ ID NO: 112 - protein, 9 aa, Artificial Sequence
  Synthetic construct
YTFTSYYMS

SEQ ID NO: 113 - protein, 14 aa, Artificial Sequence
  Synthetic construct
WVRQAPGQGLEWMG

SEQ ID NO: 114 - protein, 17 aa, Artificial Sequence
  Synthetic construct
IINPSGGSTSYAQKFQG

SEQ ID NO: 115 - protein, 30 aa, Artificial Sequence
  Synthetic construct
RVTMTRDTSTSTVYMELSSLRSEDTAVYYC

SEQ ID NO: 116 - protein, 13 aa, Artificial Sequence
  Synthetic construct
ARVRYGYADGMDV

SEQ ID NO: 117 - protein, 11 aa, Artificial Sequence
  Synthetic construct
WGQGTTVTVSS

SEQ ID NO: 118 - protein, 107 aa, Artificial Sequence
  Synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQVYHLPFTFGGGTKVEIK

SEQ ID NO: 119 - DNA, 321 bp, Artificial Sequence
  Synthetic construct
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcagcaagtataccacctccctttcacttttggcggagggaccaaggttgagatcaaa

SEQ ID NO: 120 - protein, 23 aa, Artificial Sequence
  Synthetic construct
DIQMTQSPSSLSASVGDRVTITC

SEQ ID NO: 121 - protein, 11 aa, Artificial Sequence
  Synthetic construct
RASQSISSYLN

SEQ ID NO: 122 - protein, 15 aa, Artificial Sequence
  Synthetic construct
WYQQKPGKAPKLLIY

SEQ ID NO: 123 - protein, 7 aa, Artificial Sequence
  Synthetic construct
GASSLQS

SEQ ID NO: 124 - protein, 32 aa, Artificial Sequence; identical to SEQ ID NO: 52
  Synthetic construct

SEQ ID NO: 125 - protein, 9 aa, Artificial Sequence
  Synthetic construct
QQVYHLPFT

SEQ ID NO: 126 - protein, 10 aa, Artificial Sequence
  Synthetic construct
FGGGTKVEIK

SEQ ID NO: 127 - protein, 121 aa, Artificial Sequence
  Synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDPLYQDAPFDYWGQGTLVTVSS

SEQ ID NO: 128 - DNA, 363 bp, Artificial Sequence
  Synthetic construct
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagttctgtgaccgccgcagacacggcggtgtactactgcgccagagatcctttgtaccaagacgctcccttcgactattggggacagggtacattggtcaccgtctcctca

SEQ ID NO: 129 - protein, 26 aa, Artificial Sequence
  Synthetic construct
QLQLQESGPGLVKPSETLSLTCTVSG

SEQ ID NO: 130 - protein, 11 aa, Artificial Sequence
  Synthetic construct
GSISSSSYYWG

SEQ ID NO: 131 - protein, 14 aa, Artificial Sequence
  Synthetic construct
WIRQPPGKGLEWIG

SEQ ID NO: 132 - protein, 16 aa, Artificial Sequence
  Synthetic construct
SIYYSGSTYYNPSLKS

SEQ ID NO: 133 - protein, 30 aa, Artificial Sequence
  Synthetic construct
RVTISVDTSKNQFSLKLSSVTAADTAVYYC

SEQ ID NO: 134 - protein, 13 aa, Artificial Sequence
  Synthetic construct
ARDPLYQDAPFDY

SEQ ID NO: 135 - protein, 11 aa, Artificial Sequence
  Synthetic construct
WGQGTLVTVSS

SEQ ID NO: 136 - protein, 106 aa, Artificial Sequence
  Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRANFPTFGGGTKVEIK

SEQ ID NO: 137 - DNA, 318 bp, Artificial Sequence
  Synthetic construct
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagagagccaacttccctacttttggcggagggaccaaggttgagatcaaa

SEQ ID NO: 138 - protein, 23 aa, Artificial Sequence
  Synthetic construct
EIVLTQSPATLSLSPGERATLSC

SEQ ID NO: 139 - protein, 11 aa, Artificial Sequence
  Synthetic construct
RASQSVSSYLA

SEQ ID NO: 140 - protein, 15 aa, Artificial Sequence
  Synthetic construct
WYQQKPGQAPRLLIY

SEQ ID NO: 141 - protein, 7 aa, Artificial Sequence
  Synthetic construct
DASNRAT

SEQ ID NO: 142 - protein, 32 aa, Artificial Sequence
  Synthetic construct
GIPARFSGSGSGTDFTLTISSLEPEDFAVYYC

SEQ ID NO: 143 - protein, 8 aa, Artificial Sequence
  Synthetic construct
QQRANFPT

SEQ ID NO: 144 - protein, 10 aa, Artificial Sequence
  Synthetic construct
FGGGTKVEIK

SEQ ID NO: 145 - protein, 124 aa, Artificial Sequence
  Synthetic construct
QVQLQESGPGLVKPSETLSLTCAVSGYSISSGYYWAWIRQPPGKGLEWIGSIYHSGSTYYNPSLKSRVTISVDTSKNQFSLKLSSVTAADTAVYYCARQGYYYGSSGSVDFDLWGRGTLVTVSS

SEQ ID NO: 146 - DNA, 372 bp, Artificial Sequence
  Synthetic construct
caggtgcagctgcaggagtcgggcccaggactggtgaagccttcggagaccctgtccctcacctgcgctgtctctggttactccatcagcagtggttactactgggcttggatccggcagcccccagggaaggggctggagtggattgggagtatctatcatagtgggagcacctactacaacccgtccctcaagagtcgagtcaccatatcagtagacacgtccaagaaccagttctccctgaagctgagttctgtgaccgccgcagacacggcggtgtactactgcgccaggcagggatactactacggcagcagcggcagtgtagacttcgacctatgggggagaggtaccttggtcaccgtctcctca

SEQ ID NO: 147 - protein, 26 aa, Artificial Sequence
  Synthetic construct
QVQLQESGPGLVKPSETLSLTCAVSG

SEQ ID NO: 148 - protein, 10 aa, Artificial Sequence
  Synthetic construct
YSISSGYYWA

SEQ ID NO: 149 - protein, 14 aa, Artificial Sequence
  Synthetic construct
WIRQPPGKGLEWIG

SEQ ID NO: 150 - protein, 16 aa, Artificial Sequence
  Synthetic construct
SIYHSGSTYYNPSLKS

SEQ ID NO: 151 - protein, 30 aa, Artificial Sequence; identical to SEQ ID NO: 133
  Synthetic construct

SEQ ID NO: 152 - protein, 17 aa, Artificial Sequence
  Synthetic construct
ARQGYYYGSSGSVDFDL

SEQ ID NO: 153 - protein, 11 aa, Artificial Sequence
  Synthetic construct
WGRGTLVTVSS

SEQ ID NO: 154 - protein, 108 aa, Artificial Sequence
  Synthetic construct
DIQMTQSPSSVSASVGDRVTITCRASQGISSWLAWYQQKPGKAPKLLIYAASNLQSGVPSRFSGSGSGTDFTLTISSLQPEDFATYYCQQANSLPPWTFGGGTKVEIK

SEQ ID NO: 155 - DNA, 324 bp, Artificial Sequence
  Synthetic construct
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccaatttgcaaagtggggtcccatcaaggttcagcggcagtggatctgggacagatttcactctcaccatcagcagcctgcagcctgaagattttgcaacttattactgtcaacaggcaaatagtctccctccttggacttttggcggagggaccaaggttgagatcaaa

SEQ ID NO: 156 - protein, 23 aa, Artificial Sequence
  Synthetic construct
DIQMTQSPSSVSASVGDRVTITC

SEQ ID NO: 157 - protein, 11 aa, Artificial Sequence
  Synthetic construct
RASQGISSWLA

SEQ ID NO: 158 - protein, 15 aa, Artificial Sequence
  Synthetic construct
WYQQKPGKAPKLLIY

SEQ ID NO: 159 - protein, 7 aa, Artificial Sequence
  Synthetic construct
AASNLQS

SEQ ID NO: 160 - protein, 32 aa, Artificial Sequence; identical to SEQ ID NO: 52
  Synthetic construct

SEQ ID NO: 161 - protein, 10 aa, Artificial Sequence
  Synthetic construct
QQANSLPPWT

SEQ ID NO: 162 - protein, 10 aa, Artificial Sequence
  Synthetic construct
FGGGTKVEIK

SEQ ID NO: 163 - protein, 125 aa, Artificial Sequence
  Synthetic construct
QVQLVQSGAEVKKPGASVKVSCKASGYTFTSYAISWVRQAPGQGLEWMGWISAYNGNTNYAQKLQGRVTMTTDTSTSTAYMELRSLRSDDTAVYYCARDLSSFWSGDVLGAFDIWGQGTMVTVSS

SEQ ID NO: 164 - DNA, 375 bp, Artificial Sequence
  Synthetic construct
caggttcagctggtgcagtctggagctgaggtgaagaagcctggggcctcagtgaaggtctcctgcaaggcttctggttacacctttaccagctatgccatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggcggtgtactactgcgcaagggatttgtctagcttctggagcggagacgtgttaggagccttcgacatatggggtcagggtacaatggtcaccgtctcctca

SEQ ID NO: 165 - protein, 26 aa, Artificial Sequence
  Synthetic construct
QVQLVQSGAEVKKPGASVKVSCKASG

SEQ ID NO: 166 - protein, 9 aa, Artificial Sequence
  Synthetic construct
YTFTSYAIS

SEQ ID NO: 167 - protein, 14 aa, Artificial Sequence
  Synthetic construct
WVRQAPGQGLEWMG

SEQ ID NO: 168 - protein, 17 aa, Artificial Sequence
  Synthetic construct
WISAYNGNTNYAQKLQG

SEQ ID NO: 169 - protein, 30 aa, Artificial Sequence
  Synthetic construct
RVTMTTDTSTSTAYMELRSLRSDDTAVYYC

SEQ ID NO: 170 - protein, 18 aa, Artificial Sequence
  Synthetic construct
ARDLSSFWSGDVLGAFDI

SEQ ID NO: 171 - protein, 11 aa, Artificial Sequence
  Synthetic construct
WGQGTMVTVSS

SEQ ID NO: 172 - protein, 106 aa, Artificial Sequence
  Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSVPPRTFGGGTKVEIK

SEQ ID NO: 173 - DNA, 318 bp, Artificial Sequence
  Synthetic construct
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagcgtcccccccaggacttttggcggagggaccaaggttgagatcaaa

SEQ ID NO: 174 - protein, 23 aa, Artificial Sequence
  Synthetic construct
DIQMTQSPSSLSASVGDRVTITC

SEQ ID NO: 175 - protein, 11 aa, Artificial Sequence
  Synthetic construct
RASQSISSYLN

SEQ ID NO: 176 - protein, 15 aa, Artificial Sequence
  Synthetic construct
WYQQKPGKAPKLLIY

SEQ ID NO: 177 - protein, 7 aa, Artificial Sequence
  Synthetic construct
AASSLQS

SEQ ID NO: 178 - protein, 32 aa, Artificial Sequence; identical to SEQ ID NO: 52
  Synthetic construct

SEQ ID NO: 179 - protein, 8 aa, Artificial Sequence
  Synthetic construct
QQSVPPRT

SEQ ID NO: 180 - protein, 10 aa, Artificial Sequence
  Synthetic construct
FGGGTKVEIK

SEQ ID NO: 181 - protein, 125 aa, Artificial Sequence; identical to SEQ ID NO: 163
  Synthetic construct

SEQ ID NO: 182 - DNA, 375 bp, Artificial Sequence; identical to SEQ ID NO: 164
  Synthetic construct

SEQ ID NO: 183 - protein, 26 aa, Artificial Sequence
  Synthetic construct
QVQLVQSGAEVKKPGASVKVSCKASG

SEQ ID NO: 184 - protein, 9 aa, Artificial Sequence
  Synthetic construct
YTFTSYAIS

SEQ ID NO: 185 - protein, 14 aa, Artificial Sequence
  Synthetic construct
WVRQAPGQGLEWMG

SEQ ID NO: 186 - protein, 17 aa, Artificial Sequence
  Synthetic construct
WISAYNGNTNYAQKLQG

SEQ ID NO: 187 - protein, 30 aa, Artificial Sequence; identical to SEQ ID NO: 169
  Synthetic construct

SEQ ID NO: 188 - protein, 18 aa, Artificial Sequence
  Synthetic construct
ARDLSSFWSGDVLGAFDI

SEQ ID NO: 189 - protein, 11 aa, Artificial Sequence
  Synthetic construct
WGQGTMVTVSS

SEQ ID NO: 190 - protein, 106 aa, Artificial Sequence
  Synthetic construct
EIVMTQSPATLSVSPGERATLSCRASQSVSSNLAWYQQKPGQAPRLLIYGASTRATGIPARFSGSGSGTEFTLTISSLQSEDFAVYYCQQHANHITFGGGTKVEIK

SEQ ID NO: 191 - DNA, 318 bp, Artificial Sequence
  Synthetic construct
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagcacgccaatcacatcacttttggcggagggaccaaggttgagatcaaa

SEQ ID NO: 192 - protein, 23 aa, Artificial Sequence
  Synthetic construct
EIVMTQSPATLSVSPGERATLSC

SEQ ID NO: 193 - protein, 11 aa, Artificial Sequence
  Synthetic construct
RASQSVSSNLA

SEQ ID NO: 194 - protein, 15 aa, Artificial Sequence
  Synthetic construct
WYQQKPGQAPRLLIY

SEQ ID NO: 195 - protein, 7 aa, Artificial Sequence
  Synthetic construct
GASTRAT

SEQ ID NO: 196 - protein, 32 aa, Artificial Sequence
  Synthetic construct
GIPARFSGSGSGTEFTLTISSLQSEDFAVYYC

SEQ ID NO: 197 - protein, 8 aa, Artificial Sequence
  Synthetic construct
QQHANHIT

SEQ ID NO: 198 - protein, 10 aa, Artificial Sequence
  Synthetic construct
FGGGTKVEIK

SEQ ID NO: 199 - protein, 124 aa, Artificial Sequence
  Synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARASDSYGVGLYYGMDVWGQGTTVTVSS

SEQ ID NO: 200 - DNA, 372 bp, Artificial Sequence
  Synthetic construct
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggcggtgtactactgcgctagggcatctgactcctacggagtgggcctctactacggaatggacgtatggggccagggaacaactgtcaccgtctcctca

SEQ ID NO: 201 - protein, 26 aa, Artificial Sequence
  Synthetic construct
QVQLVQSGAEVKKPGASVKVSCKASG

SEQ ID NO: 202 - protein, 9 aa, Artificial Sequence
  Synthetic construct
YTFTSYYMH

SEQ ID NO: 203 - protein, 14 aa, Artificial Sequence
  Synthetic construct
WVRQAPGQGLEWMG

SEQ ID NO: 204 - protein, 17 aa, Artificial Sequence
  Synthetic construct
IINPSGGSTSYAQKFQG

SEQ ID NO: 205 - protein, 30 aa, Artificial Sequence; identical to SEQ ID NO: 115
  Synthetic construct

SEQ ID NO: 206 - protein, 17 aa, Artificial Sequence
  Synthetic construct
ARASDSYGVGLYYGMDV

SEQ ID NO: 207 - protein, 11 aa, Artificial Sequence
  Synthetic construct
WGQGTTVTVSS

SEQ ID NO: 208 - protein, 108 aa, Artificial Sequence
  Synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQYYVSPLTFGGGTKVEIK

SEQ ID NO: 209 - DNA, 324 bp, Artificial Sequence
  Synthetic construct
gaaattgtgttgacgcagtctccaggcaccctgtctttgtctccaggggaaagagccaccctctcctgcagggccagtcagagtgttaggagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtactacgtcagtcctctcacttttggcggagggaccaaggttgagatcaaa

SEQ ID NO: 210 - protein, 23 aa, Artificial Sequence
  Synthetic construct
EIVLTQSPGTLSLSPGERATLSC

SEQ ID NO: 211 - protein, 12 aa, Artificial Sequence
  Synthetic construct
RASQSVRSSYLA

SEQ ID NO: 212 - protein, 15 aa, Artificial Sequence
  Synthetic construct
WYQQKPGQAPRLLIY

SEQ ID NO: 213 - protein, 7 aa, Artificial Sequence
  Synthetic construct
GASSRAT

SEQ ID NO: 214 - protein, 32 aa, Artificial Sequence; identical to SEQ ID NO: 16
  Synthetic construct

SEQ ID NO: 215 - protein, 9 aa, Artificial Sequence
  Synthetic construct
QQYYVSPLT

SEQ ID NO: 216 - protein, 10 aa, Artificial Sequence
  Synthetic construct
FGGGTKVEIK

SEQ ID NO: 217 - DNA, 2978 bp, Homo sapiens
cgtcctatctgcagtcggctactttcagtggcagaagaggccacatctgcttcctgtaggccctctgggcagaagcatgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttagcaaccagaggcatcttctggaagatacacttttgtctttgctattatagatgaatatataagcagctgtactctccatcagtgctgcgtgtgtgtgtgtgtgtgtatgtgtgtgtgtgttcagttgagtgaataaatgtcatcctcttctccatcttcatttccttggccttttcgttctattccattttgcattatggcaggcctagggtgagtaacgtggatcttgatcataaatgcaaaattaaaaaatatcttgacctggttttaaatctggcagtttgagcagatcctatgtctctgagagacacattcctcataatggccagcattttgggctacaaggttttgtggttgatgatgaggatggcatgactgcagagccatcctcatctcattttttcacgtcattttcagtaactttcactcattcaaaggcaggttataagtaagtcctggtagcagcctctatggggagatttgagagtgactaaatcttggtatctgccctcaagaacttacagttaaatggggagacaatgttgtcatgaaaaggtattatagtaaggagagaaggagacatacacaggccttcaggaagagacgacagtttggggtgaggtagttggcataggcttatctgtgatgaagtggcctgggagcaccaaggggatgttgaggctagtctgggaggagcaggagttttgtctagggaacttgtaggaaattcttggagctgaaagtcccacaaagaaggccctggcaccaagggagtcagcaaacttcagattttattctctgggcaggcatttcaagtttccttttgctgtgacatactcatccattagacagcctgatacaggcctgtagcctcttccggccgtgtgtgctggggaagccccaggaaacgcacatgcccacacagggagccaagtcgtagcatttgggccttgatctaccttttctgcatcaatacactcttgagcctttgaaaaaagaacgtttcccactaaaaagaaaatgtggatttttaaaatagggactcttcctaggggaaaaaggggggctgggagtgatagagggtttaaaaaataaacaccttcaaactaacttcttcgaacccttttattcactccctgacgactttgtgctggggttggggtaactgaaccgcttatttctgtttaattgcattcaggctggatcttagaagacttttatccttccaccatctctctcagaggaatgagcggggaggttggatttactggtgactgattttctttcatgggccaaggaactgaaagagaatgtgaagcaaggttgtgtcttgcgcatggttaaaaataaagcattgtcctgcttcctaagacttagactggggttgacaattgttttagcaacaagacaattcaactatttctcctaggatttttattattattattttttcacttttctaccaaatgggttacataggaagaatgaactgaaatctgtccagagctccaagtcctttggaagaaagattagatgaacgtaaaaatgttgttgtttgctgtggcagtttacagcatttttcttgcaaaattagtgcaaatctgttggaaatagaacacaattcacaaattggaagtgaactaaaatgtaatgacgaaaagggagtagtgttttgatttggaggaggtgtatattcggcagaggttggactgagagttgggtgttatttaacataattatggtaattgggaaacatttataaacactattgggatggtgataaaatacaaaagggcctatagatgttagaaatgggtcaggttactgaaatgggattcaatttgaaaaaaatttttttaaatagaactcactgaactagattctcctctgagaaccagagaagaccatttcatagttggattcctggagacatgcgctatccaccacgtagccactttccacatgtggccatcaaccacttaagatggggttagtttaaatcaagatgtgctgttataattggtataagcataaaatcacactagattctggagatttaatatgaataataagaatactatttcagtagttttggtatattgtgtgtcaaaaatgataatattttggatgtattgggtgaaataaaatattaacattaaaaaaaaaaa

SEQ ID NO: 218 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 219 - protein, 312 aa, Macaca fascicularis
MAFLVAPPMQFVYLLKTLCVFNMVFAKPGFSETVFSHRLSFTVLSAVGYFRWQKRPHLLPVSPLGRSMRWCLFLIWAQGLRQAPLASGMMTGTIETTGNISAKKGGSVILQCHLSSTMAQVTQVNWEQHDHSLLAIRNAELGWHIYPAFKDRVAPGPGLGLTLQSLTMNDTGEYFCTYHTYPDGTYRGRIFLEVLESSVAEHSARFQIPLLGAMAMMLVVICIAVIVVVVLARKKKSLRIHSVESGLQRKSTGQEEQIPSAPSPPGSCVQAEAAPAGLCGEQQGDDCAELHDYFNVLSYRSLGSCSFFTETG

SEQ ID NO: 220 - protein, 241 aa, Mus musculus
MHGWLLLVWVQGLIQAAFLATGATAGTIDTKRNISAEEGGSVILQCHFSSDTAEVTQVDWKQQDQLLAIYSVDLGWHVASVFSDRVVPGPSLGLTFQSLTMNDTGEYFCTYHTYPGGIYKGRIFLKVQESSVAQFQTAPLGGTMAAVLGLICLMVTGVTVLARKKSIRMHSIESGLGRTEAEPQEWNLRSLSSPGSPVQTQTAPAGPCGEQAEDDYADPQEYFNVLSYRSLESFIAVSKTG

SEQ ID NO: 221 - protein, 9 aa, Artificial Sequence
  Synthetic construct
FTFTDYYMD

SEQ ID NO: 222 - protein, 19 aa, Artificial Sequence
  Synthetic construct
RTRNKVNSYYTEYAASVKG

SEQ ID NO: 223 - protein, 19 aa, Artificial Sequence
  Synthetic construct
ARGQYYYGSDRRGYYYMDV

SEQ ID NO: 224 - protein, 9 aa, Artificial Sequence
  Synthetic construct
GTFLSSAIS

SEQ ID NO: 225 - protein, 17 aa, Artificial Sequence
  Synthetic construct
SLIPYFGTANYAQKFQG

SEQ ID NO: 226 - protein, 9 aa, Artificial Sequence
  Synthetic construct
GTFSAWAIS

SEQ ID NO: 227 - protein, 17 aa, Artificial Sequence
  Synthetic construct
SIIPYFGKANYAQKFQG

SEQ ID NO: 228 - protein, 18 aa, Artificial Sequence
  Synthetic construct
ARGPSEVSGILGYVWFDP

SEQ ID NO: 229 - protein, 17 aa, Artificial Sequence
  Synthetic construct
SIIPLFGKANYAQKFQG

SEQ ID NO: 230 - protein, 18 aa, Artificial Sequence
  Synthetic construct
ARGPSEVKGILGYVWFDP

SEQ ID NO: 231 - protein, 9 aa, Artificial Sequence
  Synthetic construct
GTFREYAIS

SEQ ID NO: 232 - protein, 17 aa, Artificial Sequence
  Synthetic construct
GIHPIFGTARYAQKFQG

SEQ ID NO: 233 - protein, 9 aa, Artificial Sequence
  Synthetic construct
GTFSDYPIS

SEQ ID NO: 234 - protein, 17 aa, Artificial Sequence
  Synthetic construct
GIIPIVGGANYAQKFQG

SEQ ID NO: 235 - protein, 14 aa, Artificial Sequence
  Synthetic construct
TRQSTWHKLYGTDV

SEQ ID NO: 236 - protein, 14 aa, Artificial Sequence
  Synthetic construct
TRQSTWHKLFGTDV

SEQ ID NO: 237 - protein, 14 aa, Artificial Sequence
  Synthetic construct
ARQSTWHKVYGTDV

SEQ ID NO: 238 - protein, 17 aa, Artificial Sequence
  Synthetic construct
WISAYNGNTKYAQKLQG

SEQ ID NO: 239 - protein, 9 aa, Artificial Sequence
  Synthetic construct
YTFTSYPIG

SEQ ID NO: 240 - protein, 17 aa, Artificial Sequence
  Synthetic construct
WISSYNGNTNYAQKLQG

SEQ ID NO: 241 - protein, 18 aa, Artificial Sequence
  Synthetic construct
ARGASSFWSGDVLGAFDI

SEQ ID NO: 242 - protein, 18 aa, Artificial Sequence
  Synthetic construct
ARDLKSFWSGDVLGAFDI

SEQ ID NO: 243 - protein, 9 aa, Artificial Sequence
  Synthetic construct
YTFTSYAIA

SEQ ID NO: 244 - protein, 18 aa, Artificial Sequence
  Synthetic construct
ARSGSSFWSGDVLGAFDI

SEQ ID NO: 245 - protein, 128 aa, Artificial Sequence
  Synthetic construct
EVQLVESGGGLVQPGGSLRLSCAASGFTFTDYYMDWVRQAPGKGLEWVGRTRNKVNSYYTEYAASVKGRFTISRDDSKNSLYLQMNSLKTEDTAVYYCARGQYYYGSDRRGYYYMDVWGQGTTVTVSS

SEQ ID NO: 246 - protein, 125 aa, Artificial Sequence
  Synthetic construct
QVQLVQSGAEVKKPGSSVKVSCKASGGTFLSSAISWVRQAPGQGLEWMGSLIPYFGTANYAQKFQGRVTITADESTSTAYMELSSLRSEDTAVYYCARGPSEVGAILGYVWFDPWGQGTLVTVSS

SEQ ID NO: 247 - protein, 125 aa, Artificial Sequence
  Synthetic construct
QVQLVQSGAEVKKPGSSVKVSCKASGGTFSAWAISWVRQAPGQGLEWMGSIIPYFGKANYAQKFQGRVTITADESTSTAYMELSSLRSEDTAVYYCARGPSEVSGILGYVWFDPWGQGTLVTVSS

SEQ ID NO: 248 - protein, 125 aa, Artificial Sequence
  Synthetic construct
QVQLVQSGAEVKKPGSSVKVSCKASGGTFLSSAISWVRQAPGQGLEWMGSIIPLFGKANYAQKFQGRVTITADESTSTAYMELSSLRSEDTAVYYCARGPSEVKGILGYVWFDPWGQGTLVTVSS

SEQ ID NO: 249 - protein, 125 aa, Artificial Sequence
  Synthetic construct
QVQLVQSGAEVKKPGSSVKVSCKASGGTFLSSAISWVRQAPGQGLEWMGSIIPYFGKANYAQKFQGRVTITADESTSTAYMELSSLRSEDTAVYYCARGPSEVKGILGYVWFDPWGQGTLVTVSS

SEQ ID NO: 250 - protein, 121 aa, Artificial Sequence
  Synthetic construct
QVQLVQSGAEVKKPGSSVKVSCKASGGTFREYAISWVRQAPGQGLEWMGGIHPIFGTARYAQKFQGRVTITADESTSTAYMELSSLRSEDTAVYYCTRQSTWHKLYGTDVWGQGTTVTVSS

SEQ ID NO: 251 - protein, 121 aa, Artificial Sequence
  Synthetic construct
QVQLVQSGAEVKKPGSSVKVSCKASGGTFSDYPISWVRQAPGQGLEWMGGIIPIVGGANYAQKFQGRVTITADESTSTAYMELSSLRSEDTAVYYCTRQSTWHKLFGTDVWGQGTTVTVSS

SEQ ID NO: 252 - protein, 121 aa, Artificial Sequence
  Synthetic construct
QVQLVQSGAEVKKPGSSVKVSCKASGGTFSDYPISWVRQAPGQGLEWMGGIIPIVGGANYAQKFQGRVTITADESTSTAYMELSSLRSEDTAVYYCARQSTWHKVYGTDVWGQGTTVTVSS

SEQ ID NO: 253 - protein, 125 aa, Artificial Sequence
  Synthetic construct
QVQLVQSGAEVKKPGASVKVSCKASGYTFTSYAISWVRQAPGQGLEWMGWISAYNGNTKYAQKLQGRVTMTTDTSTSTAYMELRSLRSDDTAVYYCARDLSSFWSGDVLGAFDIWGQGTMVTVSS

SEQ ID NO: 254 - protein, 125 aa, Artificial Sequence
  Synthetic construct
QVQLVQSGAEVKKPGASVKVSCKASGYTFTSYPIGWVRQAPGQGLEWMGWISSYNGNTNYAQKLQGRVTMTTDTSTSTAYMELRSLRSDDTAVYYCARDLSSFWSGDVLGAFDIWGQGTMVTVSS

SEQ ID NO: 255 - protein, 125 aa, Artificial Sequence
  Synthetic construct
QVQLVQSGAEVKKPGASVKVSCKASGYTFTSYPIGWVRQAPGQGLEWMGWISSYNGNTNYAQKLQGRVTMTTDTSTSTAYMELRSLRSDDTAVYYCARGASSFWSGDVLGAFDIWGQGTMVTVSS

SEQ ID NO: 256 - protein, 125 aa, Artificial Sequence
  Synthetic construct
QVQLVQSGAEVKKPGASVKVSCKASGYTFTSYPIGWVRQAPGQGLEWMGWISSYNGNTNYAQKLQGRVTMTTDTSTSTAYMELRSLRSDDTAVYYCARDLKSFWSGDVLGAFDIWGQGTMVTVSS

SEQ ID NO: 257 - protein, 125 aa, Artificial Sequence
  Synthetic construct
QVQLVQSGAEVKKPGASVKVSCKASGYTFTSYAIAWVRQAPGQGLEWMGWISAYNGNTNYAQKLQGRVTMTTDTSTSTAYMELRSLRSDDTAVYYCARSGSSFWSGDVLGAFDIWGQGTMVTVSS

SEQ ID NO: 258 - protein, 15 aa, Homo sapiens
ICNADLGWHISPSFK

SEQ ID NO: 259 - protein, 13 aa, Artificial Sequence
  Synthetic construct
ILQCHLSSTTAQV

SEQ ID NO: 260 - protein, 15 aa, Artificial Sequence
  Synthetic construct
CIYHTYPDGTYTGRI

SEQ ID NO: 261 - protein, 11 aa, Artificial Sequence
  Synthetic construct
SFKDRVAPGPG

SEQ ID NO: 262 - protein, 7 aa, Artificial Sequence
  Synthetic construct
TTAQVTQ

SEQ ID NO: 263 - protein, 14 aa, Artificial Sequence
  Synthetic construct
SFKDRVAPGPGLGL

SEQ ID NO: 264 - protein, 33 aa, Artificial Sequence
  Synthetic construct
  MISC_FEATURE (5)..(5): Xaa = Cys-acm
  MISC_FEATURE (19)..(19): Xaa = Cys-acm
CILQXHLSSTTAQVTQCIXNADLGWHISPSFKC

SEQ ID NO: 265 - protein, 7 aa, Artificial Sequence
  Synthetic construct
ADHIQRY

SEQ ID NO: 266 - protein, 33 aa, Artificial Sequence
  Synthetic construct
  MISC_FEATURE (19)..(19): Xaa = Cys-acm
CKDRVAPGPGLGLTLQCIXNADLGWHISPSFKC

SEQ ID NO: 267 - protein, 5 aa, Artificial Sequence
  Synthetic construct
TAQVT

SEQ ID NO: 268 - protein, 4 aa, Artificial Sequence
  Synthetic construct
GWHI

SEQ ID NO: 269 - protein, 7 aa, Artificial Sequence
  Synthetic construct
PGPGLGL

SEQ ID NO: 270 - protein, 5 aa, Artificial Sequence
  Synthetic construct
KDRVA